SEQUENCE LISTING (29 sequences)

SEQ ID NO: 1 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - protein, 725 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 745 aa, synthetic construct
MAADGYLPDWLEDTLSEGIRQWWKLKPGPPPPKAAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSALALGETTRPAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 746 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLALGETTRPAAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - DNA, 2214 bp, synthetic construct
  source 1..2214: mol_type = other DNA
atggatatgagagtccccgcccaactcctcggtttgcttctcctttggttgagaggggccagatgcgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgccgcgccaagcgcagcggctccggtgcccccgtgaagcaaaccctgaatttcgatctgctgaagttggcgggcgacgtggaaagcaatcccggacccatggatatgcgcgtgccggcccagttgctgggactgctgctcctgtggctgcggggagccagatgccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagtga

SEQ ID NO: 7 - DNA, 2403 bp, synthetic construct
  source 1..2403: mol_type = other DNA
atggatatgagagtccccgcccaactcctcggtttgcttctcctttggttgagaggggccagatgcgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgcggaggggggggatcaggagggggtggatcaggtggtggaggatctgactacaaagaccatgatggagactacaaggaccatgacattgactacaaggatgatgatgacaagcgcgccaagcgcagcggctccggtgcccccgtgaagcaaaccctgaatttcgatctgctgaagttggcgggcgacgtggaaagcaatcccggacccatggatatgcgcgtgccggcccagttgctgggactgctgctcctgtggctgcggggagccagatgccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagggagggggtggctcaggcggaggcggctcgggcggcgggggcagcgaacagaagctgatctccgaagaggacctgtgataa

SEQ ID NO: 8 - DNA, 2403 bp, synthetic construct
  source 1..2403: mol_type = other DNA
atggacatgagagtgcctgcacaacttcttggacttctgttgctctggttgagaggagcccgctgtgacatccagatgacccagtccccaagcagcctgtcagcctcagtgggagatagggtcaccattacttgcaaagccagtgaaaatgtggacacctatgtgtcctggtaccagcagaagcctggcaaagcacccaagctcctcatctatggagcctccaaccggtacactggggtgccctcccggttctcgggatcaggatcgggtactgactttaccttcactattagcagcctgcaacctgaggacattgccacataccactgtggacagtcccactcctaccccctgacctttgggcagggcaccaagctggagatcaagagaactgtggcagccccctctgtgttcatcttccctccctcggatgaacagctgaagtcaggcactgcttctgtggtctgcctcctcaacaacttctacccaagagaggccaaggtccagtggaaagtggacaatgccctgcaaagtggcaacagccaggaatcagtgactgaacaggactccaaggacagcacttacagcctgtcctccaccctcaccctgtccaaggctgactatgagaagcacaaggtctatgcctgtgaagtgacccaccagggcttgagcagcccagtgaccaagagcttcaacagaggggagtgtggaggggggggatcaggagggggtggatcaggtggtggaggatctgactacaaagaccatgatggagactacaaggaccatgacattgactacaaggatgatgatgacaagagagccaagaggtctggatcaggggccccagtgaagcagaccctcaactttgacctcctgaagctggctggagatgtggaatccaaccctggtcccatggatatgagggtgccagcccagctcctgggcctcctgctcctgtggctgaggggtgccagatgccaagtgcagctggtgcagtcgggtgctgaagtgaaaaagcctggagcctcagtcaaagtgtcctgcaaggcctcaggctacaccttcactgacttctacatggactgggtccgccaagcccctggacagcggctggagtggatgggctacatctacccccacaatgctggcaccacctacaaccagcagttcactgggagagtcaccatcacagtggacaagagtgcctccactgcatacatggaactgagcagcctcagatcagaggacactgcagtctactactgtgctaggagagggggctttgactttgactactggggtcaagggacccttgtcactgtgtcctcagcctccaccaagggcccctcagtgttccccctggccccctcctccaaatccacctctgggggaactgcagccctgggctgcctggtcaaggactacttccctgagcctgtgactgtctcctggaactcaggagccctgacatcaggagtgcacaccttccctgctgtgctgcaaagctcaggcttgtactccctgtcctctgtggtcactgtgccaagctccagcctaggtacccagacctacatctgcaatgtcaaccacaagccctccaacaccaaggtggacaagaaagtggagcccaagtcctgtgacaagacccatacttgccctccctgccctgcccctgccttggctggaggtccatcagtgttcctgttcccacccaagcccaaggataccctcatgatctcccgcactcctgaagtgacctgtgtggtggtggatgtgtcccatgaggaccctgaagtcaagttcaattggtatgtggatggagtggaagtgcacaatgccaagaccaagcctagggaagaacagtacaacagcacctacagggttgtgtcagtgctgactgtgctgcaccaggattggctcaatggcaaagagtacaagtgcaaagtatccaacaaggccctgcctgcccccattgaaaagaccatctccaaggccaagggccagcccagggaaccccaggtctacaccctgcccccttcccgggaggagatgaccaagaaccaagtctccctgacttgccttgtgaagggtttctacccctcggacattgcagtggaatgggagtccaatggccagccagagaacaactacaagaccacccctcctgtgctggactcagatggctccttcttcctgtactccaagttaactgtggacaagtccagatggcagcagggcaatgtgttcagctgctcagtgatgcatgaagccctgcacaaccactacacccagaagtccctgtccctgagcccaggaaagggtggagggggctcagggggaggaggctctggagggggaggctcagagcaaaagctgatctcagaggaagatctgtgataa

SEQ ID NO: 9 - DNA, 2403 bp, synthetic construct
  source 1..2403: mol_type = other DNA
atggatatgagagtccctgcacaattactgggcctgctgctgctttggttgaggggagccagatgtgacattcagatgactcagtccccctcctccttgtcggcctcagtgggggacagggtcaccatcacctgtaaagcctcagagaatgtggatacctatgtctcctggtaccagcagaagcctggaaaggccccaaagctcctgatctatggtgccagcaaccggtacactggtgtcccctcccgcttctcgggatcaggatcgggcactgacttcacattcaccatttcctcccttcaacctgaggacattgccacctaccattgtggacagagccactcctacccccttacctttggccagggtaccaagctggaaatcaagagaacagtggcagccccctctgtgttcatctttcccccttcggatgagcagctgaagagtggcactgcttcggtggtctgcctccttaacaacttctacccccgggaagccaaggtccagtggaaagtggacaatgccctgcagtcgggcaacagccaagagtcagtgactgagcaagactccaaggactccacctactccctctcctccaccctcaccctgagcaaggctgactatgagaagcacaaggtctatgcctgtgaagtgacccaccagggcctgagcagcccagtgaccaagagcttcaacaggggagagtgtggaggggggggatcaggagggggtggatcaggtggtggaggatctgactacaaagaccatgatggagactacaaggaccatgacattgactacaaggatgatgatgacaagagagccaagagatctggttcgggagcccctgtgaagcaaaccctcaactttgacctactgaagctggctggagatgtggaatccaacccaggtcccatggacatgagggtgccagcccagctcctggggctgctcctcctctggctgcggggagctaggtgccaggtccaactggtgcagtcaggggcagaagtcaagaagcctggtgccagtgtgaaagtgtcctgcaaagcctcaggatacaccttcactgacttctacatggactgggttcgccaggcacctggccagaggctggagtggatgggttacatctacccccacaacgctggaaccacctacaaccagcagttcactggaagagtcaccattactgtggataagtctgccagcactgcctacatggaactcagctccctgagatcagaggacactgctgtgtactactgtgcccgccggggaggctttgactttgactactggggccagggcaccctggtcactgtgtcctcggcctccactaagggtccgtcagtgttcccattggccccaagctccaagagcacctcaggaggcactgctgccctgggctgcttggtcaaggactacttccctgagcctgtgactgtgtcctggaactcaggggccctcacctcgggagtgcacaccttccctgcggtgctccagagctcaggactgtacagcctgagctcagtggtcactgtcccttcctcctccctggggacccagacctacatctgcaatgtcaaccataagccctccaacaccaaagtagacaagaaagtggagcccaagtcctgtgataagacccacacttgccctccctgcccggcccctgccctggctggaggaccctcagtgttcctgttcccacccaagcccaaggataccctgatgatctcccgcactcctgaagtgacctgtgtggtggtggatgtgtcccatgaagatcctgaagtcaagttcaattggtatgtggatggagtggaagtgcacaatgcaaagaccaagccaagagaagaacagtacaacagcacctaccgggtggtgtcagtgctgactgtgctgcaccaggactggcttaatggaaaggagtacaagtgcaaagtgtccaacaaggccctgcctgcccccattgaaaagaccatctccaaggccaagggccagcccagggaaccccaagtgtacaccctgcccccgtcccgggaagagatgaccaagaaccaagtgtccctgacttgcctggtcaagggcttctacccctcggacattgcagtggagtgggaaagcaatggccagccagagaacaactacaagacaacccctcctgtgctggactcagatggctccttcttcctgtactccaagctcactgtggacaagtccagatggcaacagggcaatgtgttctcctgctctgtgatgcatgaggccctccacaaccactacacccaaaagtccttgagcctgtccccggggaagggagggggtggctcagggggagggggctctgggggtggaggctcagaacagaagctgatctcagaagaggacctgtgataa

SEQ ID NO: 10 - DNA, 2403 bp, synthetic construct
  source 1..2403: mol_type = other DNA
atggatatgagagtgcctgcccaactgttaggattgctgctgctttggttgaggggagccagatgtgacatccagatgacccagtccccatcctccctgtctgcatcagtgggggatagagtgaccattacttgcaaagcctctgagaatgtggatacctatgtgtcctggtatcaacagaagccaggaaaagcccccaaactcctcatctatggtgcaagcaacaggtacactggagtgcccagcagattctcaggcagtggttcaggaactgacttcaccttcactatcagcagcctgcagcctgaggacattgccacctaccattgtggacagagccactcctacccactcacctttggccagggcactaagctggagatcaagaggactgtggcagccccctcagtctttatcttcccacctagtgatgaacagctgaagtcaggcactgcctcagtggtttgcctgctcaacaacttctacccaagagaagccaaggtccagtggaaggtggacaatgcccttcagtctggcaacagccaagagtcagtgactgaacaggactccaaggactccacctactccctctcctccaccctcaccctgagcaaggcagactatgagaagcacaaggtctatgcctgtgaagtgacccaccagggcctgagcagccctgtcaccaagagcttcaacaggggagagtgtggaggggggggatcaggagggggtggatcaggtggtggaggatctgactacaaagaccatgatggagactacaaggaccatgacattgactacaaggatgatgatgacaagagagccaagagatcaggctctggagcccctgtgaagcagaccctcaactttgatctgctgaagctggctggggatgtggagagcaaccctggccccatggacatgagagtccctgcccaattgctgggcctccttctcctgtggcttaggggtgctaggtgccaagtgcagctggtgcagtcaggggctgaagtgaaaaagcctggagcctcagtgaaagtgtcctgcaaggcttcagggtacaccttcactgacttctacatggattgggtcagacaggccccaggacagaggctggagtggatgggctacatctacccccacaatgcagggaccacctacaaccagcagttcactggaagagtgactatcacagtggataagtcagcctccactgcctacatggagctgtccagcctgagatcagaggacactgcagtctactactgtgccagaaggggaggctttgactttgactactggggacagggcaccctggtcactgtctcctcagcaagcaccaagggtccttcagtgttccccttggccccctcctccaagtccacctcagggggaactgcagccctggggtgcctggtcaaggactacttccctgagccagtcactgtgtcctggaactcaggggccctcacctctggagtgcacaccttccctgctgtgctgcaaagctcaggcctgtacagcctgagctcagtggtcactgtgccctcctcctccctgggtacccaaacctacatctgcaatgtcaaccacaagccctccaacaccaaagtggacaagaaagtggagcccaagtcctgtgacaagacccacacatgccctccctgccctgcccctgccctggctggaggtccctcagtgttcctgttcccacccaagcccaaggacacactgatgatctccaggactcctgaagtgacctgtgtggtagtggatgtgtcccatgaagatcctgaagtcaagttcaattggtatgtggatggagtggaagtgcacaatgccaagaccaagcccagagaagaacagtacaacagcacctacagagtggtgtcagtcctcactgtgctgcaccaggactggctcaatggaaaggagtacaagtgcaaagtgtccaacaaggccctgcctgcccccattgaaaagaccatctccaaggccaagggacagcccagggaaccccaggtctacaccctaccaccatccagggaggaaatgaccaagaaccaagtgtccttgacttgccttgtgaagggcttctacccctctgacattgctgtggaatgggaatccaatggtcaacctgagaacaactacaagaccacccctcctgtgctggacagtgatggctccttcttcctgtactccaagctgactgtggacaagtccagatggcagcagggcaatgtgttcagctgctcagtgatgcatgaagccctccacaaccactacacccagaagtccctctccctgtcccctgggaagggagggggtggctcagggggagggggctcaggaggtggaggctcagagcagaagctgatttctgaagaggacctgtgataa

SEQ ID NO: 11 - DNA, 2196 bp, synthetic construct
  source 1..2196: mol_type = other DNA
atggatatgagagtccccgcccaactcctcggtttgcttctcctttggttgagaggggccagatgcgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgccgcgccaagcgcagcggctccggtgaggggcgggggagcctgctgacatgcggcgatgtggaggagaacccgggcccgatggatatgcgcgtgccggcccagttgctgggactgctgctcctgtggctgcggggagccagatgccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagtga

SEQ ID NO: 12 - DNA, 2385 bp, synthetic construct
  source 1..2385: mol_type = other DNA
atggatatgagagtccccgcccaactcctcggtttgcttctcctttggttgagaggggccagatgcgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgcggaggggggggatcaggagggggtggatcaggtggtggaggatctgactacaaagaccatgatggagactacaaggaccatgacattgactacaaggatgatgatgacaagcgcgccaagcgcagcggctccggtgaggggcgggggagcctgctgacatgcggcgatgtggaggagaacccgggcccgatggatatgcgcgtgccggcccagttgctgggactgctgctcctgtggctgcggggagccagatgccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagggagggggtggctcaggcggaggcggctcgggcggcgggggcagcgaacagaagctgatctccgaagaggacctgtgataa

SEQ ID NO: 13 - DNA, 2385 bp, synthetic construct
  source 1..2385: mol_type = other DNA
atggacatgagagtgcctgcacaacttcttggacttctgttgctctggttgagaggagcccgctgtgacatccagatgacccagtccccaagcagcctgtcagcctcagtgggagatagggtcaccattacttgcaaagccagtgaaaatgtggacacctatgtgtcctggtaccagcagaagcctggcaaagcacccaagctcctcatctatggagcctccaaccggtacactggggtgccctcccggttctcgggatcaggatcgggtactgactttaccttcactattagcagcctgcaacctgaggacattgccacataccactgtggacagtcccactcctaccccctgacctttgggcagggcaccaagctggagatcaagagaactgtggcagccccctctgtgttcatcttccctccctcggatgaacagctgaagtcaggcactgcttctgtggtctgcctcctcaacaacttctacccaagagaggccaaggtccagtggaaagtggacaatgccctgcaaagtggcaacagccaggaatcagtgactgaacaggactccaaggacagcacttacagcctgtcctccaccctcaccctgtccaaggctgactatgagaagcacaaggtctatgcctgtgaagtgacccaccagggcttgagcagcccagtgaccaagagcttcaacagaggggagtgtggaggggggggatcaggagggggtggatcaggtggtggaggatctgactacaaagaccatgatggagactacaaggaccatgacattgactacaaggatgatgatgacaagagagccaagaggtctggatcaggggaagggaggggtagcctgctcacctgtggagacgtggaggaaaaccctggccccatggatatgagggtgccagcccagctcctgggcctcctgctcctgtggctgaggggtgccagatgccaagtgcagctggtgcagtcgggtgctgaagtgaaaaagcctggagcctcagtcaaagtgtcctgcaaggcctcaggctacaccttcactgacttctacatggactgggtccgccaagcccctggacagcggctggagtggatgggctacatctacccccacaatgctggcaccacctacaaccagcagttcactgggagagtcaccatcacagtggacaagagtgcctccactgcatacatggaactgagcagcctcagatcagaggacactgcagtctactactgtgctaggagagggggctttgactttgactactggggtcaagggacccttgtcactgtgtcctcagcctccaccaagggcccctcagtgttccccctggccccctcctccaaatccacctctgggggaactgcagccctgggctgcctggtcaaggactacttccctgagcctgtgactgtctcctggaactcaggagccctgacatcaggagtgcacaccttccctgctgtgctgcaaagctcaggcttgtactccctgtcctctgtggtcactgtgccaagctccagcctaggtacccagacctacatctgcaatgtcaaccacaagccctccaacaccaaggtggacaagaaagtggagcccaagtcctgtgacaagacccatacttgccctccctgccctgcccctgccttggctggaggtccatcagtgttcctgttcccacccaagcccaaggataccctcatgatctcccgcactcctgaagtgacctgtgtggtggtggatgtgtcccatgaggaccctgaagtcaagttcaattggtatgtggatggagtggaagtgcacaatgccaagaccaagcctagggaagaacagtacaacagcacctacagggttgtgtcagtgctgactgtgctgcaccaggattggctcaatggcaaagagtacaagtgcaaagtatccaacaaggccctgcctgcccccattgaaaagaccatctccaaggccaagggccagcccagggaaccccaggtctacaccctgcccccttcccgggaggagatgaccaagaaccaagtctccctgacttgccttgtgaagggtttctacccctcggacattgcagtggaatgggagtccaatggccagccagagaacaactacaagaccacccctcctgtgctggactcagatggctccttcttcctgtactccaagttaactgtggacaagtccagatggcagcagggcaatgtgttcagctgctcagtgatgcatgaagccctgcacaaccactacacccagaagtccctgtccctgagcccaggaaagggtggagggggctcagggggaggaggctctggagggggaggctcagagcaaaagctgatctcagaggaagatctgtgataa

SEQ ID NO: 14 - DNA, 2385 bp, synthetic construct
  source 1..2385: mol_type = other DNA
atggatatgagagtccctgcacaattactgggcctgctgctgctttggttgaggggagccagatgtgacattcagatgactcagtccccctcctccttgtcggcctcagtgggggacagggtcaccatcacctgtaaagcctcagagaatgtggatacctatgtctcctggtaccagcagaagcctggaaaggccccaaagctcctgatctatggtgccagcaaccggtacactggtgtcccctcccgcttctcgggatcaggatcgggcactgacttcacattcaccatttcctcccttcaacctgaggacattgccacctaccattgtggacagagccactcctacccccttacctttggccagggtaccaagctggaaatcaagagaacagtggcagccccctctgtgttcatctttcccccttcggatgagcagctgaagagtggcactgcttcggtggtctgcctccttaacaacttctacccccgggaagccaaggtccagtggaaagtggacaatgccctgcagtcgggcaacagccaagagtcagtgactgagcaagactccaaggactccacctactccctctcctccaccctcaccctgagcaaggctgactatgagaagcacaaggtctatgcctgtgaagtgacccaccagggcctgagcagcccagtgaccaagagcttcaacaggggagagtgtggaggggggggatcaggagggggtggatcaggtggtggaggatctgactacaaagaccatgatggagactacaaggaccatgacattgactacaaggatgatgatgacaagagagccaagagatctggttcgggagagggaaggggctccctcctgacttgtggagatgtggaagagaaccctggccccatggacatgagggtgccagcccagctcctggggctgctcctcctctggctgcggggagctaggtgccaggtccaactggtgcagtcaggggcagaagtcaagaagcctggtgccagtgtgaaagtgtcctgcaaagcctcaggatacaccttcactgacttctacatggactgggttcgccaggcacctggccagaggctggagtggatgggttacatctacccccacaacgctggaaccacctacaaccagcagttcactggaagagtcaccattactgtggataagtctgccagcactgcctacatggaactcagctccctgagatcagaggacactgctgtgtactactgtgcccgccggggaggctttgactttgactactggggccagggcaccctggtcactgtgtcctcggcctccactaagggtccgtcagtgttcccattggccccaagctccaagagcacctcaggaggcactgctgccctgggctgcttggtcaaggactacttccctgagcctgtgactgtgtcctggaactcaggggccctcacctcgggagtgcacaccttccctgcggtgctccagagctcaggactgtacagcctgagctcagtggtcactgtcccttcctcctccctggggacccagacctacatctgcaatgtcaaccataagccctccaacaccaaagtagacaagaaagtggagcccaagtcctgtgataagacccacacttgccctccctgcccggcccctgccctggctggaggaccctcagtgttcctgttcccacccaagcccaaggataccctgatgatctcccgcactcctgaagtgacctgtgtggtggtggatgtgtcccatgaagatcctgaagtcaagttcaattggtatgtggatggagtggaagtgcacaatgcaaagaccaagccaagagaagaacagtacaacagcacctaccgggtggtgtcagtgctgactgtgctgcaccaggactggcttaatggaaaggagtacaagtgcaaagtgtccaacaaggccctgcctgcccccattgaaaagaccatctccaaggccaagggccagcccagggaaccccaagtgtacaccctgcccccgtcccgggaagagatgaccaagaaccaagtgtccctgacttgcctggtcaagggcttctacccctcggacattgcagtggagtgggaaagcaatggccagccagagaacaactacaagacaacccctcctgtgctggactcagatggctccttcttcctgtactccaagctcactgtggacaagtccagatggcaacagggcaatgtgttctcctgctctgtgatgcatgaggccctccacaaccactacacccaaaagtccttgagcctgtccccggggaagggagggggtggctcagggggagggggctctgggggtggaggctcagaacagaagctgatctcagaagaggacctgtgataa

SEQ ID NO: 15 - DNA, 2385 bp, synthetic construct
  source 1..2385: mol_type = other DNA
atggatatgagagtgcctgcccaactgttaggattgctgctgctttggttgaggggagccagatgtgacatccagatgacccagtccccatcctccctgtctgcatcagtgggggatagagtgaccattacttgcaaagcctctgagaatgtggatacctatgtgtcctggtatcaacagaagccaggaaaagcccccaaactcctcatctatggtgcaagcaacaggtacactggagtgcccagcagattctcaggcagtggttcaggaactgacttcaccttcactatcagcagcctgcagcctgaggacattgccacctaccattgtggacagagccactcctacccactcacctttggccagggcactaagctggagatcaagaggactgtggcagccccctcagtctttatcttcccacctagtgatgaacagctgaagtcaggcactgcctcagtggtttgcctgctcaacaacttctacccaagagaagccaaggtccagtggaaggtggacaatgcccttcagtctggcaacagccaagagtcagtgactgaacaggactccaaggactccacctactccctctcctccaccctcaccctgagcaaggcagactatgagaagcacaaggtctatgcctgtgaagtgacccaccagggcctgagcagccctgtcaccaagagcttcaacaggggagagtgtggaggggggggatcaggagggggtggatcaggtggtggaggatctgactacaaagaccatgatggagactacaaggaccatgacattgactacaaggatgatgatgacaagagagccaagagatcaggctctggagaggggaggggcagcctgctcacctgtggagatgtggaggaaaaccctggccccatggacatgagagtccctgcccaattgctgggcctccttctcctgtggcttaggggtgctaggtgccaagtgcagctggtgcagtcaggggctgaagtgaaaaagcctggagcctcagtgaaagtgtcctgcaaggcttcagggtacaccttcactgacttctacatggattgggtcagacaggccccaggacagaggctggagtggatgggctacatctacccccacaatgcagggaccacctacaaccagcagttcactggaagagtgactatcacagtggataagtcagcctccactgcctacatggagctgtccagcctgagatcagaggacactgcagtctactactgtgccagaaggggaggctttgactttgactactggggacagggcaccctggtcactgtctcctcagcaagcaccaagggtccttcagtgttccccttggccccctcctccaagtccacctcagggggaactgcagccctggggtgcctggtcaaggactacttccctgagccagtcactgtgtcctggaactcaggggccctcacctctggagtgcacaccttccctgctgtgctgcaaagctcaggcctgtacagcctgagctcagtggtcactgtgccctcctcctccctgggtacccaaacctacatctgcaatgtcaaccacaagccctccaacaccaaagtggacaagaaagtggagcccaagtcctgtgacaagacccacacatgccctccctgccctgcccctgccctggctggaggtccctcagtgttcctgttcccacccaagcccaaggacacactgatgatctccaggactcctgaagtgacctgtgtggtagtggatgtgtcccatgaagatcctgaagtcaagttcaattggtatgtggatggagtggaagtgcacaatgccaagaccaagcccagagaagaacagtacaacagcacctacagagtggtgtcagtcctcactgtgctgcaccaggactggctcaatggaaaggagtacaagtgcaaagtgtccaacaaggccctgcctgcccccattgaaaagaccatctccaaggccaagggacagcccagggaaccccaggtctacaccctaccaccatccagggaggaaatgaccaagaaccaagtgtccttgacttgccttgtgaagggcttctacccctctgacattgctgtggaatgggaatccaatggtcaacctgagaacaactacaagaccacccctcctgtgctggacagtgatggctccttcttcctgtactccaagctgactgtggacaagtccagatggcagcagggcaatgtgttcagctgctcagtgatgcatgaagccctccacaaccactacacccagaagtccctctccctgtcccctgggaagggagggggtggctcagggggagggggctcaggaggtggaggctcagagcagaagctgatttctgaagaggacctgtgataa

SEQ ID NO: 16 - DNA, 2160 bp, synthetic construct
  source 1..2160: mol_type = other DNA
atgcccctgttgctgctgcttccacttctctgggctggtgcccttgctgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgccgcgccaagcgcagcggctccggtgaggggcgggggagcctgctgacatgcggcgatgtggaggagaacccgggcccgatgccactcctccttctgttgcctctcctctgggccggagctctggcacaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagtga

SEQ ID NO: 17 - RNA, 27 nt, synthetic construct
  source 1..27: mol_type = other RNA
cgcaatcagtgaatgcttatacatccg

SEQ ID NO: 18 - DNA, 2160 bp, synthetic construct
  source 1..2160: mol_type = other DNA
atgccactcctccttctgttgcctctcctctgggccggagctctggcacaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagcgcgccaagcgcagcggctccggtgaggggcgggggagcctgctgacatgcggcgatgtggaggagaacccgggcccgatgcccctgttgctgctgcttccacttctctgggctggtgcccttgctgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgctga

SEQ ID NO: 19 - DNA, 2208 bp, synthetic construct
  source 1..2208: mol_type = other DNA
atgcctatgggatccttgcaacctctggccaccctctacctccttggcatgcttgtggcatcagtgctggctgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgccgcgccaagcgcagcggctccggtgaggggcgggggagcctgctgacatgcggcgatgtggaggagaacccgggcccgatgcctatgggatccctccaacccctcgccaccctctatctcttgggaatgcttgttgccagtgtgcttgcccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagtga

SEQ ID NO: 20 - DNA, 2208 bp, synthetic construct
  source 1..2208: mol_type = other DNA
atgcctatgggatccctccaacccctcgccaccctctatctcttgggaatgcttgttgccagtgtgcttgcccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagcgcgccaagcgcagcggctccggtgaggggcgggggagcctgctgacatgcggcgatgtggaggagaacccgggcccgatgcctatgggatccttgcaacctctggccaccctctacctccttggcatgcttgtggcatcagtgctggctgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgctga

SEQ ID NO: 21 - DNA, 2220 bp, synthetic construct
  source 1..2220: mol_type = other DNA
atggtgtcatactgggatactggagtcctgctttgtgccctgctttcctgcctgctgttgactggctcctcctcgggagacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgccgcgccaagcgcagcggctccggtgaggggcgggggagcctgctgacatgcggcgatgtggaggagaacccgggcccgatggtgtcatattgggatactggagtccttctctgtgctttgctctcctgcctcctcctcactggatccagctcgggacaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagtga

SEQ ID NO: 22 - DNA, 2220 bp, synthetic construct
  source 1..2220: mol_type = other DNA
atggtgtcatattgggatactggagtccttctctgtgctttgctctcctgcctcctcctcactggatccagctcgggacaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagcgcgccaagcgcagcggctccggtgaggggcgggggagcctgctgacatgcggcgatgtggaggagaacccgggcccgatggtgtcatactgggatactggagtcctgctttgtgccctgctttcctgcctgctgttgactggctcctcctcgggagacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgctga

SEQ ID NO: 23 - DNA, 2196 bp, synthetic construct
  source 1..2196: mol_type = other DNA
atggatatgcgcgtgccggcccagttgctgggactgctgctcctgtggctgcggggagccagatgccaagtgcagcttgtgcagtccggtgccgaagtcaagaaacctggagcctccgtcaaagtgtcctgcaaggcctcgggctacaccttcaccgacttctacatggactgggtccgacaggcgccagggcagcgcctggagtggatgggctatatctacccgcacaacgcaggaaccacctacaaccagcagttcactggaagagtgaccattaccgtcgataagtcagccagcaccgcgtacatggaactgtcctctctccggtccgaggatactgctgtgtactactgtgcgcggcggggcggcttcgactttgactactggggacaggggactctggtcactgtgtcctccgcatccaccaagggtccttcagtgttcccattggccccgagctcaaagtccacatcagggggaactgccgctctcggatgcctcgtgaaggattactttcccgaacccgtgaccgtgtcctggaactctggcgcgctcacctccggggtgcataccttccccgcggtgctgcagagctccgggctgtactccctgtcgagcgtggtcaccgtgccatcgtcgtccctgggaactcagacctacatctgcaacgtgaaccacaagccatccaacaccaaggtcgacaagaaggtcgagccgaagtcctgcgataagactcatacttgcccgccgtgccctgctccggcccttgccgggggccctagcgtgttcctcttcccacctaaaccgaaggacaccctcatgatttcccgcactcctgaagtgacctgtgtggtagtggacgtgtcccatgaggaccccgaggtcaagttcaattggtacgtggacggcgtggaagtgcacaacgccaagaccaagccgagggaggaacagtacaactcgacttaccgggtcgtgtccgtgctgactgtgctgcaccaggactggctcaacggcaaagagtataagtgcaaagtgtccaacaaggccctgcccgctccgattgaaaagactattagcaaggccaagggacagcctcgggagccgcaagtgtacaccctgccgccatcacgggaggaaatgaccaagaaccaagtttccctgacgtgcctggtcaaagggttctacccctcggacatcgcggtggagtgggaatccaacggtcaacctgaaaacaactacaagaccactccgcccgtgctcgactccgacggatcgttcttcttgtactccaagctgaccgtggacaagagccgctggcagcagggcaacgtgttctcatgttccgtgatgcatgaagccctgcataaccactacactcagaagtccctgagcctgtcccccggaaagcgcgccaagcgcagcggctccggtgaggggcgggggagcctgctgacatgcggcgatgtggaggagaacccgggcccgatggatatgagagtccccgcccaactcctcggtttgcttctcctttggttgagaggggccagatgcgacatccaaatgacccagtcgccgtcgtcgctgtcggcgtccgtcggtgatcgcgtgaccatcacttgtaaagcctcggagaacgtggacacctatgtgtcctggtaccagcagaagccgggaaaggcccccaagctcttaatctacggcgcatccaaccgctacaccggagtcccgtcccgcttctccggctcgggctcgggaactgacttcaccttcaccatttcgtcactgcaaccagaggatattgctacctaccactgtggacagtcacactcctaccccctgacctttggtcaagggaccaagctcgaaatcaagcggactgtggccgccccttccgtgttcatcttccctccttccgatgagcagctcaagagcggaactgcaagcgtcgtgtgcctgctgaacaacttttacccccgggaagccaaagtgcagtggaaagtcgacaatgccctgcaatccggaaacagtcaggaaagcgtgaccgaacaggactcgaaggactccacgtactccctttcctccacgctaaccctgagcaaggcagactatgagaagcacaaggtctacgcatgcgaagtgacacaccagggcctgagcagccctgtgaccaagagcttcaacaggggagagtgctga

SEQ ID NO: 24 - protein, 447 aa, synthetic construct
QVQLVQSGAEVKKPGASVKVSCKASGYTFTDFYMDWVRQAPGQRLEWMGYIYPHNAGTTYNQQFTGRVTITVDKSASTAYMELSSLRSEDTAVYYCARRGGFDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, synthetic construct
DIQMTQSPSSLSASVGDRVTITCKASENVDTYVSWYQQKPGKAPKLLIYGASNRYTGVPSRFSGSGSGTDFTFTISSLQPEDIATYHCGQSHSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 16 aa, synthetic construct
MPLLLLLPLLWAGALA

SEQ ID NO: 27 - protein, 22 aa, synthetic construct
MDMRVPAQLLGLLLLWLRGARC

SEQ ID NO: 28 - protein, 24 aa, synthetic construct
MPMGSLQPLATLYLLGMLVASVLA

SEQ ID NO: 29 - protein, 26 aa, synthetic construct
MVSYWDTGVLLCALLSCLLLTGSSSG

(19) United States
(12) Patent Application Publication
(10) Pub. No.: US 2026/0043046 A1
(43) Pub. Date: Feb. 12, 2026

(54) CASSETTES OF ANTI-COMPLEMENT COMPONENT 3 ANTIBODY, VECTORIZATION AND THERAPUTIC APPLICATION

(71) Applicant: Adverum Biotechnologies, Inc., Redwood City, CA (US)

(72) Inventors: Ruslan Grishanin, Redwood City, CA (US); Jie Ma, Redwood City, CA (US); Sing Ying Cheung, Redwood City, CA (US); Kellie Schaefer-Swale, Redwood City, CA (US); Brigit E. Riley, Redwood City, CA (US)

(73) Assignee: Adverum Biotechnologies, Inc., Redwood City, CA (US)

(21) Appl. No.: 18/846,879

(22) PCT Filed: Mar. 15, 2023
(86) PCT No.: PCT/US2023/064421

§ 371 Date: Sep. 13, 2024

Related U.S. Application Data
(60) Provisional application No. 63/320,782, filed on Mar. 17, 2022.

(51) Int. Cl. C12N 15/86; A61K 38/17; A61K 39/00; A61K 48/00; C07K 16/18

(52) U.S. Cl. CPC C12N 15/86; A61K 38/179; A61K 48/005; A61K 48/0075; C07K 16/18; A61K 2039/505; C07K 2317/92; C12N 2750/14143

(57) ABSTRACT

Provided herein are methods for expression of anti-C3 molecule by administering a recombinant adeno-associated virus (rAAV) virion in an amount capable of blocking C3 activity in the eye.

### CROSS-REFERENCE TO RELATED APPLICATIONS

This application is a 371 National Stage filing of and claims the benefit under 35 U.S.C. § 120 of International Application No. PCT/US2023/064421 filed Mar. 15, 2023, which claims priority under 35 U.S.C. § 119(e) to U.S. Provisional Application No. 63/320,782 filed Mar. 17, 2022, both of which are hereby incorporated by reference in their entirety.

### STATEMENT REGARDING THE SEQUENCE LISTING

The official copy of the Sequence Listing is submitted concurrently with the specification as an WIPO Standard ST.26 formatted XML file with file name “17234.035WO1.xml”, a creation date of Mar. 13, 2023, and a size of 69,259 bytes. This Sequence Listing filed via USPTO Patent Center is part of the specification and is incorporated in its entirety by reference herein.

### BACKGROUND OF THE INVENTION

Dry AMD with geographic atrophy (GA) is characterized by retinal pigment epithelium (RPE) and photoreceptor death leading to vision loss, which affects the quality of life in the ageing population world-wide. This disease is characterized by localized sharply demarcated atrophy of outer retinal tissue, RPE and choriocapillaris. It starts typically in the perifoveal region and expands to involve the fovea with time, leading to central scotomas and permanent loss of visual acuity. It is bilateral in most cases. Over 8 million people are affected worldwide with GA, approximately 20% of all individuals with AMD. The incidence of GA is expected to rise as the age-burden of developed countries is increasing. Currently, no treatment exists for GA and although the cause of dry AMD is unknown, activation of components of the complement cascade have been implicated through GWAS (genome-wide association study) and proteomic studies. Complement component 3 (C3) plays a central role in the complement system. Intravitreally injected C3 inhibitors have been demonstrated to reduce progression of GA growth. However, slow progression of this disease, the need for multiple intravitreal (IVT) injections and non-compliance may threaten success of such therapies.

Therefore, there is a need in the art for a longer lasting treatment for GA.

### SUMMARY OF THE INVENTION

To address the barriers discussed above, rAAV (recombinant adeno-associated virus) particles for intraocular expression of a C3-blocking full-length humanized antibody optimized for IVT delivery may be used. An IVT-delivered gene therapy offers the potential for long term suppression of GA progression while avoiding the need for multiple injections.

Optimal transgene architecture was determined by utilizing an iterative screening process involving evaluation of several components of the cassettes including, “self-cleaving” peptides, signals for secretion, and antibody chain orientation.

Plasmids were transfected into suspension HEK293 cells and measured for human IgG1 level, which correlates to the amount of anti-C3 antibody produced. Candidates which revealed high expression profiles were vectorized into the AAV2.7m8-based capsid and were further tested in ARPE19 cells and rabbit retinal explants. A candidate, which robustly expressed anti-C3 antibody in the ex vivo system was selected for further development.

Bilateral IVT injections (1 μL/eye, ˜2.40-2.63×10⁹ vg/eye) of the vector were administered to C57BL/6 mice, and expression of humanized anti-C3 antibody was detected 1-week post-injection, with levels exceeding 0.25 ng/50 μg of retinal protein at week 6, as determined by total protein extracted from retinas. No observable abnormalities or pathology was observed in the mouse eyes 6 weeks after IVT delivery by in vivo examination of fundus images. Furthermore, we evaluated levels of humanized C3 antibody in the eyes of non-human primates (NHP). Four cynomolgus monkeys (Macaca fascicularis) were treated bilaterally with a single dose of AAV2.7m8-pADV1176 at 1×10¹¹ vg/eye, which resulted in the intraocular expression of the antibody at levels ranging from 6-39 ng/mL in aqueous and 16-60 ng/mL in vitreous humors, measured 4-7 weeks post-injection. Findings support the candidate as an IVT-delivered gene therapy for dry AMD patients with GA.

In one aspect, the present invention provides a method for providing at least one transgene encoding a gene product to ocular cells in a subject, administering within the eye of the subject an effective amount of a composition comprising a first rAAV comprising said transgene, wherein the subject comprises a C3 blocking molecule, such as an anti-C3 antibody, wherein the transduced ocular cell expresses the gene product. In particular embodiments, the cells comprise retinal cells. In particular embodiments, the rAAV is administered by an IVT injection.

In particular embodiments of any aspect with regard to providing a gene product to ocular cells, the composition may further include a second rAAV, which may or may not be the same serotype, wherein the second rAAV comprises the same or different capsid proteins as the first rAAV, and/or the first rAAV and the second rAAV may be AAV2.7m8, AAV9.7m8, AAV2.5T.LSV1 (LSV1: Loop Swap Variant 1) or AAV2.R100. In particular embodiments of any aspect, the transgene in the first rAAV may be different from the transgene in the second rAAV. In certain embodiments, the first and/or second gene product may be a therapeutic gene product. In certain embodiments, the first gene product and the second gene product are independently selected from: an anti-angiogenic polypeptide, a vascular endothelial growth factor (VEGF)-binding protein, an anti-VEGF agent or anti-VEGF protein, or anti-C3 antibody.

In a related aspect, the present invention provides a method for treating an ocular disease or disorder, in particular GA associated with dry AMD, in a subject in need thereof, administering within the eye of the subject an effective amount of a rAAV comprising a transgene encoding a therapeutic gene product, wherein the subject comprises activation of components of the complement cascade, and wherein the transduced ocular cells express the therapeutic gene product. In particular embodiments, the cells comprise retinal cells. In particular embodiments, the rAAV is administered by an IVT injection.

In particular embodiments of any aspect with regard to treating an ocular disease, the composition may further include a second rAAV, which may or may not be the same serotype, wherein the second rAAV comprises the same or different capsid proteins as the first rAAV, and/or the first rAAV and the second rAAV may be AAV2.7m8, AAV9.7m8, AAV2.5T.LSV1 or AAV2.R100. In particular embodiments of any aspect, the transgene in the first rAAV may be different from the transgene in the second rAAV. In certain embodiments, the first and/or second gene product may be a therapeutic gene product. In certain embodiments, the first gene product and the second gene product are independently selected from: an anti-angiogenic polypeptide, a vascular endothelial growth factor (VEGF)-binding protein, an anti-VEGF agent or anti-VEGF protein, or anti-C3 antibody.

In another related aspect, the present invention provides a method for treating an ocular disease or disorder, in particular GA associated with dry AMD, in a subject in need thereof, the method comprising: (a) administering to a first eye of the subject a first effective amount of a first rAAV comprising a first transgene encoding a first gene product; (b) waiting for a period of time; and (c) administering to a second eye of the subject a second effective amount of a second rAAV comprising a second transgene encoding a second gene product, wherein the first and second effective amounts are amounts sufficient to transduce cells of the eye, and wherein the transduced cells express the first and second gene products. In particular embodiments, the cells comprise retinal cells. In particular embodiments, the rAAV is administered by an IVT injection.

In particular embodiments of any aspect, the first rAAV and the second rAAV are the same serotype, the first rAAV and the second rAAV comprise the same capsid proteins, and/or the first rAAV and the second rAAV are AAV2.7m8, AAV9.7m8, AAV2.5T.LSV1 or AAV2.R100. In particular embodiments of any aspect, the first rAAV and the second rAAV are different serotypes, the first rAAV and the second rAAV comprise different serotypes.

In particular embodiments, the period of time is selected from the following: at least one week, at least one month, at least 3 months, at least 6 months, at least one year, at least 18 months, at least two years, at least three years, or longer than three years. In certain embodiments, the subject is not administered a recombinant rAAV during the period of time.

In particular embodiments of any aspect, the first and second gene products are the same. In particular embodiments of any aspect, the first and second gene product are different. In certain embodiments, the first and/or second gene product is a therapeutic gene product. In certain embodiments, the first gene product and the second gene product are independently selected from: an anti-angiogenic polypeptide, a vascular endothelial growth factor (VEGF)-binding protein, an anti-VEGF agent or anti-VEGF protein, or anti-C3 molecule, preferably anti-C3 antibody.

In particular embodiments, the effective amount or first effective amount is at least about 1×10⁹ vg, at least about 2×10⁹ vg, at least about 2.4×10⁹ vg, at least about 2.63×10⁹ vg, at least about 1×10¹⁰ vg, at least about 1×10¹¹ vg, at least about 5×10¹¹ vg, at least about 1×10¹³ vg per eye. In some embodiments, the effective amount or first effective amount comprises up to about 2×10⁹ vg per eye of the first rAAV. In some embodiment, the effective amount or first effective amount comprises between about 1×10⁹ and 5×10¹¹ vector genomes of the first rAAV.

In particular embodiments, the effective amount or second effective amount is at least about 1×10⁹ vg, at least about 5×10¹⁰ vg, at least about 6×10¹⁰ vg, at least about 2×10¹¹ vg, at least about 5×10¹¹ vg, or at least about 1×10¹³ vg per eye.

In further embodiment, the effective amount or second effective amount is from about 5×10¹⁰ to about 2×10¹¹ vg. Still further, the effective amount or second effective amount per eye is from about 5×10¹⁰ to about 2×10¹¹ vg, including about 6×10¹⁰ vg.

The injection volume ranges from 1 μL to 100 μL per eye.

In certain embodiments of any of the aspects, the subject is not administered an immunosuppressant prior to, concurrent with, or following administration of the rAAV or first rAAV, whereas in other embodiments, the subject is administered an immunosuppressant prior to, concurrent with, or following administration of the rAAV or first rAAV.

In particular embodiments of any aspect, the administration is performed by ocular administration, retinal administration, subretinal administration and/or intravitreal administration. In particular embodiments, the administration is performed by ocular injection, retinal injection, subretinal injection and/or intravitreal injection.

In particular embodiments of any aspect, the rAAV is replication defective.

In certain aspects, disclosed herein are methods of transducing AAV2 vectors in a subject comprising administering to the subject a recombinant adeno-associated virus (rAAV) virion at an amount capable of at least partially blocking C3 activity in the subject. In some embodiments, the rAAV virion is AAV2.7m8, AAV9.7m8, AAV2.LSV1 or AAV2.R100. In some embodiments, the subject is a mammalian subject. In some embodiments, the amount of the rAAV virion administered is at least about 1×10⁹ vg, at least about 1×10¹⁰ vg, least about 1×10¹¹ vg. In some embodiments, the amount of the rAAV virion administered is at least about 5×10¹¹ vg. In some embodiments, the amount of the rAAV virion administered is at least about 1×10¹³ vg. In some embodiments, the administration is an IVT injection.

Methods for providing a transgene encoding an anti-Component 3 (C3) molecule gene product to an ocular cell in a subject are provided. The methods comprise administering to one or more sites within the eye of the subject an effective amount of a recombinant adeno-associated virus (rAAV) comprising the transgene, wherein the effective amount is an amount sufficient to at least partially block C3 activity in the subject and transduce the ocular cell, and wherein the transduced ocular cell expresses the gene product. In some aspects, the anti-C3 molecule is an anti-C3 antibody. In various aspects, the rAAV is an AAV2.7m8, AAV2.5T.LSV1 or an AAV2.R100. In an aspect of the method, the subject is a mammalian subject. In various aspects of the method the effective amount is at least about 1×10¹⁰ vg, at least about 5×10¹⁰ vg or at least about 2×10¹¹ vg. In certain aspects, the subject suffers from geographic atrophy associated with dry AMD. In some aspects, the ocular cell is a retinal cell. In some aspects, the rAAV is administered retinally, subretinally and/or intravitreally. Aspects of the methods may further comprise administering an effective amount of a second recombinant adeno-associated virus (rAAV). In some aspects, the first and second recombinant adeno-associated virus (rAAV) is administered from a single vial. In aspects of the methods, the transgene for the second rAAV is selected from the group comprising an anti-angiogenic polypeptide, a vascular endothelial growth factor (VEGF)-binding protein and an anti-VEGF agent. In some aspects, the transgene for the second rAAV encodes a soluble VEGF receptor.

In an embodiment, methods for treating an ocular disease or disorder in a subject in need thereof are provided. Methods for treating an ocular disease or disorder in a subject in need thereof comprise administering to one or more sites within the eye of the subject an effective amount of a recombinant adeno-associated virus (rAAV) comprising a transgene encoding anti-C3 gene product, wherein the effective amount is an amount sufficient to at least partially block C3 activity in the subject and transduce ocular cells within the subject and wherein the transduced ocular cells express the therapeutic gene product. In some aspects, the anti-C3 molecule is an anti-C3 antibody. In various aspects, the rAAV is an AAV2.7m8, AAV2.5T.LSV1 or an AAV2.R100. In an aspect of the method, the subject is a mammalian subject. In various aspects of the method the effective amount is at least about 1×10¹⁰ vg, at least about 5×10¹⁰ vg, at least about 6×10¹⁰ vg or at least about 2×10¹¹ vg. In some aspects, the ocular cell is a retinal cell. In some aspects, the rAAV is administered retinally, subretinally and/or intravitreally. In various aspects of the embodiments, the ocular disease or disorder is selected from the group comprising glaucoma, retinitis pigmentosa, macular degeneration, retinoschisis, Leber's Congenital Amaurosis, diabetic retinopathy, achromotopsia, geographic atrophy associated with dry AMD and color blindness. In various aspects of the embodiments, the ocular disease or disorder is selected from the group consisting of glaucoma, retinitis pigmentosa, macular degeneration, retinoschisis, Leber's Congenital Amaurosis, diabetic retinopathy, achromotopsia, geographic atrophy associated with dry AMD and color blindness. In some aspects, the ocular disease is macular degeneration. In various aspects, the macular degeneration is wet macular degeneration. In various aspects, the macular degeneration is dry macular degeneration. In some aspects, the gene product is an anti-angiogenic polypeptide, a vascular endothelial growth factor (VEGF)-binding protein, an anti-C3 molecule or an opsin protein.

Methods for treating an ocular disease or disorder in a subject in need thereof are provided. Methods for treating an ocular disease or disorder in a subject in need thereof comprise (a) administering to a first eye of the subject a first effective amount of a first rAAV comprising a first transgene encoding a first gene product, (b) waiting for a period of time and (c) administering to a second eye of the subject a second effective amount of a second rAAV comprising a second transgene encoding a second gene product, wherein the first and second effective amounts are amounts sufficient to transduces cells of the eye, and wherein the transduced cells express the first and second gene products, wherein either of the first or second rAAV expresses an anti-C3 gene product. In some aspects of the methods, the anti-C3 gene product of either the first or second rAAV is an anti-C3 antibody.

In various aspects, the first rAAV and the second rAAV are the same serotype. In aspects of the methods, the first rAAV and the second rAAV comprise the same capsid proteins. In some aspect, the first rAAV and the second rAAV are AAV2.7m8, AAV2.5T.LSV1 or AAV2.R100. In other aspects, the first rAAV and the second rAAV are different serotypes. In some aspects, the first rAAV and the second rAAV comprise different serotypes. In various aspects, the period of time is selected from the following: at least one week, at least one month, at least three months, at least six months, at least one year, at least 18 months, at least two years, at least three years or longer than three years. In some aspects, the subject is not administered a recombinant AAV during the period of time.

In various aspects, the first and second gene products are the same. In other aspects, the first and second gene products are different. In certain aspects, the first and/or second gene product is a therapeutic gene product. In some aspects, the first gene product and the second gene product are independently selected from anti-angiogenic polypeptide, a vascular endothelial growth factor (VEGF)-binding protein, an anti-C3 molecule or an opsin protein.

In various aspects of the methods, the ocular disease or disorder is glaucoma, retinitis pigmentosa, macular degeneration, retinoschisis, Leber's Congenital Amaurosis, diabetic retinopathy, achromotopsia, geographic atrophy associated with dry AMD or color blindness. In some aspects, the ocular disease is macular degeneration. In various aspects, the macular degeneration is wet macular degeneration. In various aspects, the macular degeneration is dry macular degeneration.

In various aspects of the method the first effective amount is at least about 1×10¹⁰ vg, at least about 5×10¹⁰ vg or at least about 2×10¹¹ vg. In various aspects of the method the second effective amount is at least about 1×10¹⁰ vg, at least about 5×10¹⁰ vg or at least about 2×10¹¹ vg. In aspects of the methods, the first effective amount comprises up to about 5×10¹¹ vector genomes of the first rAAV. In some aspects the first effective amount comprises between about 1×10¹¹ and about 5×10¹¹ vector genomes of the first rAAV. In some aspects, the first effective amount comprises up to about 5×10¹¹ vector genomes of the first rAAV, and the second effective amount comprises at least about 1×10¹⁰ vector genomes of the second rAAV. In certain aspects, the first effective amount is at least about 2×10⁹ vg. In aspects of the methods, the first effective amount is lower than the second effective amount.

In some aspects, the subject is not administered an immunosuppressant prior to, concurrent with or following administration of the first rAAV. In certain aspects, an immunosuppressant is not administered to the subject after administration of the first rAAV. In other aspects, an immunosuppressant is administered to the subject after the administration of the first rAAV and before or concurrent with the administration of the second rAAV. In some aspects, an immunosuppressant is administered to the subject after the administration of the second rAAV.

In aspects of the methods, the administering of the first effective amount and the administering of the second effective amount is by intraocular injection or intravitreal injection.

In certain aspects, administration of the second effective amount of the second rAAV provides a therapeutic effect in the eye of the subject.

In some aspects of the methods, the first effective amount and the second effective amount are administered to the same eye of the subject. In other aspects of the methods, the first effective amount and the second effective amount are administered to different eyes of the subject.

In various aspects of the methods, the first effective amount and the second effective amount are treating the same ocular disease or disorder. In other aspects of the methods, the first effective amount and the second effective amount are treating different ocular diseases or disorders.

In some aspects of the methods, the first gene product and the second gene product are the same. In other aspects of the methods, the first gene product and the second gene product are different.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B. First generation candidate selection by transient transfection of plasmids in suspension HEK293 cells. (FIG. 1A) Schematic designs of cassette architecture. (FIG. 1B) A potential lead candidate pADV886 (T2A site, untagged, no CpG optimization) was selected based on the quantification (Mean±SD) of human IgG1 expression using ELISA (enzyme-linked immunoassay).

FIGS. 2A-2B. Second generation candidate selection by transient transfection of plasmids into suspension HEK293 cells. (FIG. 2A) Schematic designs of cassette architecture. (FIG. 2B) A potential lead candidate pADV1176 light chain (LC) preceding heavy chain (HC), CD33 signal sequence) was selected based on the quantification (Mean±SD) of human IgG1 expression using Cayman Therapeutic IgG1 ELISA kit.

FIGS. 3A, 3B, 3C and 3D. Comparison of pADV886 vs pADV1176 by transient transfection of plasmids in suspension HEK293 cells and confirmation of the cleavage of furin-T2A on light chains by transducing vectorized pADV886 (AAV2.7m8-pADV886) and pADV1176 (AAV2.7m8-pADV1176) in rabbit retinal explants. (FIG. 3A) Candidate pADV1176 showed higher levels of expression of full length human IgG1 in transfected cell supernatant when compared to pADV886. (FIG. 3B) Supernatants from two experiments showed candidate pADV1176 also had higher levels of heavy chain (around 50 kDa) and light chain with T2A (slight above 25 kDa) compared to pADV886 by using reduced gel. There are two bands around 25 kDa (FIG. 3B), the brighter ones above 25 kDa are light chains with un-cleaved Furin-T2A and the fader ones around 25 kDa is LC which were cleaved of Furin-T2A. (FIG. 3C) Supernatants from rabbit retinal explants transduced with AAV2.7m8-pADV886 and AAV2.7m8-pADV1176 showed two bands by using anti-light chain antibody but there was only one band at the lane of positive control. (FIG. 3D) Supernatants from rabbit retinal explants of candidate pADV886 and pADV1176 showed two bands by using anti-T2A antibody but no band at the lane of positive control. FIG. 3C and FIG. 3D confirms that Furin-T2A was not completely cleaved in the products of the two vectors. The bands at the very bottom of (FIG. 3C and FIG. 3D) are loading dye.

FIG. 4. Selection of vector candidate using cell-based assay to evaluate secreted anti-C3 antibody levels. Human retinal pigment epithelial (ARPE19) cells were infected with AAV2.7m8-pADV886 and AAV2.7m8-pADV1176 at varying multiplicity of infection (MOI); 200000, 100000, 50000, 25000, 12500, 6250, 3125 and 1562.5. AAV2.7m8-pADV1176 showed greater anti-C3 antibody levels at all MOIs tested compared to AAV2.7m8-pADV886.

FIG. 5. Quantification of human IgG1 (Mean±SD) in cell culture supernatant from rabbit retinal explants transduced with AAV2.7m8-pADV886 and AAV2.7m8-pADV1176 shows higher levels of AAV2.7m8-pADV1176, across all days measured (3, 6, 9, 12 and 15 days post-transduction). Untransduced retinal explants were used as negative control.

FIG. 6. ELISA quantification of human IgG1 levels in mouse retinas infected with AAV2.7m8-pADV886 and AAV2.7m8-pADV1176 showed higher levels of AAV2.7m8-pADV1176 compared to AAV2.7m8-pADV886 across all timepoints measured (ng/mL human IgG1 per 50 μg extracted total retinal protein) (Mean±SD).

FIGS. 7A-7D. ELISA quantification of human IgG1 levels in ocular humors of nonhuman primates infected with AAV2.7m8-pADV1176 (Mean±SD). (FIG. 7A) Human IgG1 in aqueous humor samples; (FIG. 7B) Human IgG1 in vitreous humor samples. (FIG. 7C and FIG. 7D), the peak values of human IgG1 in aqueous and vitreous humor, respectively. The peak values were determined by using the highest concentration of human IgG1 from all eyes at every timepoint.

FIGS. 8A and 8B. ELISA quantification of human IgG1 levels (Mean) in aqueous humors from Day 63 to Day 77 (FIG. 8A) and in vitreous humors on Day 77 (FIG. 8B) of nonhuman primates. LPS was administrated on Day 62.

### DETAILED DESCRIPTION OF THE INVENTION

The present disclosure provides methods of transducing ocular cells using a rAAV. Also provided are methods for promoting the expression of a transgene in ocular cells, e.g., retinal cells, in an individual, e.g., for the treatment or prophylaxis of an ocular disease or disorder. These and other objects, advantages, and features of the invention will become apparent to those persons skilled in the art upon reading the details of the compositions and methods as more fully described below.

### Definitions

As convention and as used throughout the application, a scientific format of exponential notation may be used, replacing part of the number with E+n, in which E (exponent) multiplies the preceding number by 10 to the nth power. For example, a 2-decimal scientific format displays 12345678901 as 1.23E+10, which is 1.23 times 10 to the 10th power, and may be written alternatively as 1.23×10¹⁰. Similarly, 1.23E-10 may be written alternatively as 1.23×10⁻¹⁰. In another example, a 3-decimal scientific format displays 12345678901 as 1.234E+10, which is 1.234 times 10 to the 10th power, and may be written alternatively as 1.234×10¹⁰.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors, liposomes, and other gene delivery vehicles.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077 (AAV-1), AF063497 (AAV-1), NC_001401 (AAV-2), AF043303 (AAV-2), NC_001729 (AAV-3), NC_-001829 (AAV-4), U89790 (AAV-4), NC_006152 (AAV-5), AF028704 and AAB95450 (AAV-6), AF513851 (AAV-7), AF513852 (AAV-8), and NC_006261 (AAV-8), the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303. In addition, polynucleotide sequences encoding any of the capsid proteins may be readily generated based on the amino acid sequence and the known genetic code, including codon-optimized sequences.

An “AAV virus” or “AAV viral particle” or “rAAV vector particle” or rAAV” refers to a viral particle composed of at least one AAV capsid protein (e.g., by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as a “rAAV vector particle” or simply a “rAAV vector” or “rAAV”. Production of rAAV particle necessarily includes production of rAAV vector, as such a vector is contained within a rAAV particle.

The term “replication defective” as used herein relative to an AAV viral vector of the invention means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

An “AAV variant” or “AAV mutant” as used herein refers to a viral particle composed of: a) a variant AAV capsid protein, where the variant AAV capsid protein comprises at least one amino acid difference (e.g., amino acid substitution, amino acid insertion, amino acid deletion) relative to a corresponding parental AAV capsid protein, where the AAV capsid protein does not correspond to the amino acid sequence present of a naturally occurring AAV capsid protein; and, optionally, b) a heterologous nucleic acid comprising a nucleotide sequence encoding a heterologous gene product, wherein the variant AAV capsid protein confers increased binding to heparan or a heparan sulfate proteoglycan as compared to the binding by an AAV virion comprising the corresponding parental AAV capsid protein. In certain embodiments, the variant capsid protein confers: a) increased infectivity of a retinal cell compared to the infectivity of the retinal cell by an AAV virion comprising the corresponding parental AAV capsid protein; b) altered cellular tropism as compared to the tropism of an AAV virion comprising the corresponding parental AAV capsid protein; and/or c) an increased ability to bind and/or cross the inner limiting membrane (ILM) as compared to an AAV virion comprising the corresponding parental AAV capsid protein.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with a rAAV vector.

An “infectious” virus or viral particle is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973. See also the Examples.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “gene” refers to a polynucleotide containing at least one open reading frame that is capable of encoding a particular gene product after being transcribed, and sometimes also translated. The term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “gene product” is a molecule resulting from expression of a particular gene. Gene products include, e.g., a polypeptide, an aptamer, an interfering RNA, a mRNA (messenger RNA), and the like. In particular embodiments, a “gene product” is a polypeptide, peptide, protein or interfering RNA including short interfering RNA (siRNA), miRNA (microRNA) or small hairpin RNA (shRNA). In particular embodiments, a gene product is a therapeutic gene product, e.g., a therapeutic protein.

As used herein, a “therapeutic gene” refers to a gene that, when expressed, produces a therapeutic gene product that confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that correct a genetic deficiency in a cell or mammal.

As used herein, a “transgene” is a gene that is delivered to a cell by a vector. The gene may comprise or consist of a sequence that encodes a gene product.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a gene product of interest, and is used for effecting the expression of the gene product in an intended target cell. An expression vector also comprises control elements operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, a rAAV that includes a heterologous nucleic acid encoding a heterologous gene product is a rAAV that includes a nucleic acid not normally included in a naturally-occurring, wild-type AAV, and the encoded heterologous gene product is a gene product not normally encoded by a naturally-occurring, wild-type AAV.

As used herein, the terms “polypeptide,” “peptide,” and “protein” refer to polymers of amino acids of any length. The terms also encompass an amino acid polymer that has been modified; for example, disulfide bond formation, glycosylation, lipidation, phosphorylation, or conjugation with a labeling component.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a polyadenylation sequence consists only of the promoter, polynucleotide sequence encoding the therapeutic polypeptide, and polyadenylation sequence. As another example, a polypeptide “consisting of” a recited sequence contains only the recited sequence.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs from the coding sequence and from a position downstream of a transcribed region.

A “termination signal sequence” as used herein encompasses any genetic element that causes RNA polymerase to terminate transcription, such as for example a polyadenylation signal sequence.

A “polyadenylation signal sequence” as used herein encompasses a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

The term “endogenous” as used herein with reference to a nucleotide molecule or gene product refers to a nucleic acid sequence, e.g. gene or genetic element, or gene product, e.g. RNA, protein, that is naturally occurring in or associated with a host virus or cell.

The term “native” as used herein refers to a nucleotide sequence, e.g. gene, or gene product, e.g. RNA, protein, that is present in a wildtype virus or cell. The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the “biological activity” of an “immunoglobulin”, “antibody” or fragment or variant thereof refers to the ability to bind an antigenic determinant and thereby facilitate immunological function. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The terms “administering” or “introducing”, as used herein, refer to delivery of a vector for recombinant gene or protein expression to a cell, to cells and/or organs of a subject, or to a subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

Typically, a cell is referred to as “transduced”, “infected”; “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, “activation of complement cascade” refers to activating the complement system in human adaptive immune response. Complement system plays a crucial role in the innate defense against common pathogens. Activation of complement leads to robust and efficient proteolytic cascades, which terminate in opsonization and lysis of the pathogen as well as in the generation of the classical inflammatory response through the production of potent proinflammatory molecules.

By “anti-C3 molecule” or “anti-Component 3 molecule”, it is meant a molecule such as a ligand or antibody or small molecule that acts to inhibit the effects of C3 in the host. The ligand may be a natural ligand or artificially created.

By “anti-C3 antibody” or “anti-Component 3 antibody”, it is meant an antibody which recognizes a component 3 antigen and inhibits the effect(s) of the antigen in the host (e. g., a human). As used herein, the antibody can be a single antibody or a plurality of antibodies. Preferably, the antibody is a monoclonal antibody. In certain embodiments, a neutralizing antibody can inhibit the effect(s) of the antigen of at least 1, 2, 3, 4, 5, 6, 7, 8, 9 or more difference strains of AAV. For example, the neutralizing antibody can inhibit infectivity of (e.g., cell entry), or gene expression directed by, an AAV. The non-human, human or humanized antibody may be IgG1, IgG2, IgG3, IgG4 or IgM. The human or humanized antibody fragment or antibody-like protein may be scFv or scFv-Fc.

The various compositions and methods of the invention are described below. Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing-herein is to be construed as an admission that the present invention is not entitled to antedate such publication by virtue of prior invention. Further, the dates of publication provided may be different from the actual publication dates which may need to be independently confirmed.

Unless otherwise indicated, all terms used herein have the same meaning as they would to one skilled in the art and the practice of the present invention will employ, conventional techniques of microbiology and recombinant DNA technology, which are within the knowledge of those of skill of the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. For example, while certain methods disclosed herein are exemplified by referring to rAAV, it is understood that any of these methods may be practiced using a different viral vector, including but not limited to those specifically disclosed herein. It is intended that the following claims define the scope of the invention and that methods and structures within the scope of these claims and their equivalents be covered thereby.

### Virus

In particular embodiments, methods disclosed herein are practiced using a recombinant virus or virion comprising a transgene that encodes a gene product. The recombinant virus may be used to transduce a cell, wherein the transgene expresses the gene product, in order to deliver the gene product to the cell or a subject comprising the cell.

In certain embodiments, the virus or virion is a viral vector derived from a virus, e.g., an adenovirus, an adeno-associated virus (AAV), a lentivirus, a herpes virus, an alpha virus or a retrovirus, e.g., Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) or lentivirus. While embodiments encompassing the use of adeno-associated virus are described in greater detail herein, it is expected that the ordinarily skilled artisan will appreciate that similar knowledge and skill in the art can be brought to bear on non-AAV gene therapy vectors as well. See, for example, the discussion of retroviral vectors in, e.g., U.S. Pat. Nos. 7,585,676 and 8,900,858, and the discussion of adenoviral vectors in, e.g. U.S. Pat. No. 7,858,367, the full disclosures of which are incorporated herein by reference. In certain embodiments, the recombinant virus or virion is infectious. In certain embodiments, the recombinant virion or virus is replication-competent. In certain embodiments, the recombinant virus or virion is replication-incompetent.

In particular embodiments, the virus is an adeno-associated virus (rAAV). In particular embodiments, the virus is an AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), avian AAV, bovine AAV, canine AAV, equine AAV, primate AAV, non-primate AAV, or ovine AAV.

In certain embodiment, the virus is a variant AAV. In particular embodiments, the AAV comprises a variant AAV capsid protein, e.g., the AAV capsid protein may comprises an insertion within a capsid protein, e.g., VP1, of AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV rh.10, avian AAV, bovine AAV, canine AAV, equine AAV, primate AAV, non-primate AAV, bovine AAV, AAV2/7m8, AAVShH10, AAV2.5T, AAV2.5T/7m8, AAV9/7m8, and AAV5/7m8. AAV2.5T capsid proteins and virions are described in U.S. Pat. No. 9,233,131, in which the VP1-encoding amino acid sequences of AAV2.5T (U.S. Pat. No. 9,233,131's SEQ ID NO:42 and FIGS. 10A-B). AAV2.7m8 capsid proteins are described in U.S. Pat. No. 9,193,956. AAV2.7m8 includes a 7m8 insert between amino acids 587 and 588 of the wildtype AAV2 genome. AAV2.5T/7m8 capsid proteins correspond to AAV2.5T capsid proteins further comprising a 7m8 insert. R100 capsid protein described in SEQ ID NO:42 of U.S. Patent Appl Publ No. 20200282077.

In particular embodiments, the virions and viral vectors having one or more modified or altered capsid protein exhibit: 1) increased infectivity of a retinal cell; 2) increased tissue specificity for a retinal cell as compared to one or more other cells or tissues; 3) increased binding to heparan or heparan sulfate proteoglycans and/or ILM; 4) an increased ability to infect and/or deliver a gene product across the ILM when administered intravitreally, as compared to a corresponding virion comprising its native or wild-type capsid protein instead of the modified capsid protein. In some embodiment, the retinal cell is a photoreceptor cell (e.g., rod or cone). In other cases, the retinal cell is a retinal ganglion cell. In other cases, the retinal cell is an RPE cell. In other cases, the retinal cell is a Muller cell. In other embodiments, the retinal cell is an amacrine cell, bipolar cell, or horizontal cells.

In certain embodiments, the variant capsid protein, e.g., VP1, includes an insertion of a peptide of from about 5 amino acids to about 11 amino acids in length. In particular embodiments, the insertion peptide has a length of 5 amino acids, 6 amino acids, 7 amino acids, 8 amino acids, 9 amino acids, 10 amino acids, or 11 amino acids. These insertions are collectively referred to as “7m8 insertions.”

In certain embodiments, the variant capsid protein comprises a “7m8 insertion.” In certain embodiments, the 7m8 insertion peptide comprises an amino acid sequence of any one of the formulas set forth herein. For example, a 7m8 insertion peptide can be a peptide of from 5 to 11 amino acids in length, where the insertion peptide is of Formula I:

Y₁Y₂X₁X₂X₃X₄X₅X₆X₇Y₃Y₄

where:
each of Y₁-Y₄ is independently absent or present and, if present, is independently selected from Ala, Leu, Gly, Ser, and Thr;
X₁ is absent or present and, if present, is selected from Leu, Asn, and Lys;
X₂ is selected from Gly, Glu, Ala, and Asp;
X₃ is selected from Glu, Thr, Gly, and Pro;
X₄ is selected from Thr, Ile, Gln, and Lys;
X₅ is selected from Thr and Ala;
X₆ is selected from Arg, Asn, and Thr; and
X₇ is absent or present and, if present, is selected from Pro and Asn.

As another example, a 7m8 insertion peptide can be a peptide of from 5 to 11 amino acids in length, where the insertion peptide is of Formula IIa:

Y₁Y₂X₁X₂X₃X₄X₅X₆X₇Y₃Y₄

where:
each of Y₁-Y₄ are independently absent or present and, if present, is independently selected from Ala, Leu, Gly, Ser, and Thr;
each of X₁-X₄ is any amino acid;
X₅ is Thr;
X₆ is Arg; and
and X₇ is Pro.

As another example, a 7m8 insertion peptide can be a peptide of from 5 to 11 amino acids in length, where the insertion peptide is of Formula IIb:

Y₁Y₂X₁X₂X₃X₄X₅X₆X₇Y₃Y₄

where:
each of Y₁-Y₄ is independently absent or present and, if present, is independently selected from Ala, Leu, Gly, Ser, and Thr;
X₁ is absent or present and, if present, is selected from Leu and Asn;
X₂ is absent or present and, if present, is selected from Gly and Glu;
X₃ is selected from Glu and Thr;
X₄ is selected from Thr and Ile;
X₅ is Thr;
X₆ is Arg; and
X₇ is Pro.

As another example, a 7m8 insertion peptide can be a peptide of from 5 to 11 amino acids in length, where the insertion peptide is of Formula III:

Y₁Y₂X₁X₂X₃X₄X₅X₆X₇Y₃Y₄

where:
each of Y₁-Y₄ is independent absent or present and, if present, is independently selected from Ala, Leu, Gly, Ser, and Thr;
X₁ is absent or present and, if present, is Lys;
X₂ is selected from Ala and Asp;
X₃ is selected from Gly and Pro;
X₄ is selected from Gln and Lys;
X₅ is selected from Thr and Ala;
X₆ is selected from Asn and Thr; and
X₇ is absent or present and, if present, is Asn.

As another example, a 7m8 insertion peptide can be a peptide of from 5 to 11 amino acids in length, where the insertion peptide is of Formula IV:

Y₁Y₂X₁X₂X₃X₄X₅X₆X₇Y₃Y4

where:
each of Y₁-Y₄ is independently absent or present and, if present, is independently selected from Ala, Leu, Gly, Ser, and Thr;
X₁ is absent or present, if present, is a positively charged amino acid or an uncharged amino acid; or is selected from Leu, Asn, Arg, Ala, Ser, and Lys;
X₂ is a negatively charged amino acid or an uncharged amino acid; or is selected from Gly, Glu, Ala, Val, Thr, and Asp;
X₃ is a negatively charged amino acid or an uncharged amino acid; or is selected from Glu, Thr, Gly, Asp, or Pro;
X₄ is selected from Thr, Ile, Gly, Lys, Asp, and Gln;
X₅ is a polar amino acid, an alcohol (an amino acid having a free hydroxyl group), or a hydrophobic amino acid; or is selected from Thr, Ser, Val, and Ala;
X₆ is a positively charged amino acid or an uncharged amino acid; or is selected from Arg, Val, Lys, Pro, Thr, and Asn; and
X₇ is absent or present and, if present, is a positively charged amino acid or an uncharged amino acid; or is selected from Pro, Gly, Phe, Asn, and Arg.

As non-limiting examples, the 7m8 insertion peptide can comprise or consist of an amino acid sequence selected from LGETTRP, NETITRP, KAGQANN, KDPKTTN, KDTDTTR, RAGGSVG, AVDTTKF, and STGKVPN.

In some cases, the 7m8 insertion peptide has from 1 to 4 spacer amino acids (Y₁-Y4) at the amino terminus and/or at the carboxyl terminus of any one of LGETTRP, NETITRP, KAGQANN, KDPKTTN, KDTDTTR, RAGGSVG, AVDTTKF, and STGKVPN. Suitable spacer amino acids include, but are not limited to, leucine, alanine, glycine, and serine.

For example, in some cases, a 7m8 insertion peptide has one of the following amino acid sequences: LALGETTRPA; LANETITRPA, LAKAGQANNA, LAKDPKTTNA, LAKDTDTTRA, LARAGGSVGA, LAAVDTTKFA, and LASTGKVPNA. As another example, in some cases, a 7m8 insertion peptide has one of the following amino acid sequences: AALGETTRPA; AANETITRPA, AAKAGQANNA, and AAKDPKTTNA. As yet another example, in some cases, a 7m8 insertion peptide has one of the following amino acid sequences: GLGETTRPA; GNETITRPA, GKAGQANNA, and GKDPKTTNA. As another example, in some cases, an insertion peptide comprises one of KDTDTTR, RAGGSVG, AVDTTKF, and STGKVPN, flanked on the C-terminus by AA and on the N-terminus by A; or comprises one of KDTDTTR, RAGGSVG), AVDTTKF, and STGKVPN flanked on the C-terminus by G and on the N-terminus by A. In certain embodiments, the 7m8 is a random sequence of five to 12 amino acid residues.

In certain embodiment, the 7m8 amino acid insert comprises or consists of the following amino acid sequence: LGETTRP. In particular embodiments, the 7m8 insert comprises or consists of the amino acid sequence: LALGETTRPA, or a fragment comprising at least five, at least six, at least seven, at least eight, or at least nine consecutive amino acids thereof. In particular embodiments, the 7m8 insert comprises or consists of an amino acid sequence having at least 80%, at least 85%, or at least 90% homology to the amino acid sequence: LALGETTRPA, or a fragment comprising at least five, at least six, at least seven, at least eight, or at least nine consecutive amino acids thereof. In some embodiments, a capsid protein includes an m78 insertion comprising an amino acid sequence having at least about 85%, at least about 90%, at least about 95%, at least about 98%, at least about 99%, or 100%, amino acid sequence identity to an amino acid sequence selected from LGETTRP and LALGETTRPA.

In some embodiments, a variant AAV capsid polypeptide is a chimeric capsid, e.g., the capsid comprises a portion of an AAV capsid of a first AAV serotype and a portion of an AAV capsid of a second serotype; and in some embodiments, it further comprises a 7m8 insertion relative to a corresponding parental AAV capsid protein.

The following example variants may be considered. When 7m8 is inserted in a rAAV2 (also referred to as AAV2.7m8), the amino acid sequence LALGETTRPA may be inserted into the GH loop within amino acids 570-611 of the AAV2 capsid protein, e.g., between positions 587 and 588 of the AAV2 capsid protein, VP1. When 7m8 is inserted in a rAAV1 (also referred to as AAV1.7m8), the amino acid sequence LALGETTRPA may be inserted into the GH loop within amino acids 571-612 of the AAV1 capsid protein, e.g., between amino acids 590 and 591 of the AAV1 capsid protein, VP1. When 7m8 is inserted in a rAAV5 (also referred to as AAV5.7m8), the amino acid sequence LALGETTRPA may be inserted into the GH loop within amino acids 560-601 of the AAV5 capsid protein, e.g., between amino acids 575 and 576 of the AAV5 capsid protein, VP1. When 7m8 is inserted in a rAAV6 (also referred to as AAV6.7m8), the amino acid sequence LALGETTRPA may be inserted into the GH loop within amino acids 571 to 612 of the AAV6 capsid protein, e.g., between amino acids 590 and 591 of the AAV6 capsid protein, VP1. When 7m8 is inserted in a rAAV7 (also referred to as AAV7.7m8), the amino acid sequence LALGETTRPA may be inserted into the GH loop within amino acids 572 to 613 of the AAV7 capsid protein, e.g., between amino acids 589 and 590 of the AAV7 capsid protein, VP1. When 7m8 is inserted in a rAAV8 (also referred to as AAV8.7m8), the amino acid sequence LALGETTRPA may be inserted into the GH loop within amino acids 573 to 614 of the AAV8 capsid protein, e.g., between amino acids 590 and 591 of the AAV8 capsid protein, VP1. When 7m8 is inserted in a rAAV9 (also referred to as AAV9.7m8), the amino acid sequence LALGETTRPA may be inserted into the GH loop of the AAV9 capsid protein, e.g., between amino acids 588 and 589 of the AAV9 capsid protein, VP1. When 7m8 is inserted in a rAAV10 (also referred to as AAV10.7m8), the amino acid sequence LALGETTRPA may be inserted into the GH loop within amino acids 573 to 614 of the AAV10 capsid protein, e.g., between amino acids 589 and 590 of the AAV10 capsid protein, VP1.

In some embodiments, as noted above, the rAAV comprises an AAV serotype variant, i.e., a variant AAV capsid protein that comprises at least one amino acid difference relative to a corresponding parental AAV capsid protein, e.g., a wild type AAV capsid protein, and which does not consist of an amino acid sequence present in a naturally occurring AAV capsid protein. In certain embodiments, the capsid variant is an AAV2.5T.LSV1 capsid as described in PCT/US2020/029895 and as set forth below. In other embodiments, the capsid variant is AAV2.R100 capsid as described in US Application Publication No. 20200282077 as set forth below.

Below are some exemplified variant capsid protein sequences.

Name: AAV2.7m8 VP1 Capsid Protein
(SEQ ID NO: 1)
MAADGYLPDWLEDTLSEGIRQWWKLKPGPPPPKPAERHKDDSRGLVLPGYKYLGP
FNGLDKGEPVNEADAAALEHDKAYDRQLDSGDNPYLKYNHADAEFQERLKEDTSFGGNL
GRAVFQAKKRVLEPLGLVEEPVKTAPGKKRPVEHSPVEPDSSSGTGKAGQQPARKRLNFGQ
TGDADSVPDPQPLGQPPAAPSGLGTNTMATGSGAPMADNNEGADGVGNSSGNWHCDSTW
MGDRVITTSTRTWALPTYNNHLYKQISSQSGASNDNHYFGYSTPWGYFDFNRFHCHFSPRD
WQRLINNNWGFRPKRLNFKLFNIQVKEVTQNDGTTTIANNLTSTVQVFTDSEYQLPYVLGS
AHQGCLPPFPADVFMVPQYGYLTLNNGSQAVGRSSFYCLEYFPSQMLRTGNNFTFSYTFED
VPFHSSYAHSQSLDRLMNPLIDQYLYYLSRTNTPSGTTTQSRLQFSQAGASDIRDQSRNWLP
GPCYRQQRVSKTSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNLALGETTRPARQAA
TADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTV
DTNGVYSEPRPIGTRYLTRNL
Name: AAV2.5T.LSV1 VP1 Capsid Protein
(SEQ ID NO: 2)
MAADGYLPDWLEDTLSEGIRQWWKLKPGPPPPKPAERHKDDSRGLVLPGYKYLGP
FNGLDKGEPVNEADAAALEHDKAYDRQLDSGDNPYLKYNHADAEFQERLKEDTSFGGNL
GRAVFQAKKRVLEPFGLVEEGAKTAPTGKRIDDHFPKRKKARTEEDSKPSTSSDAEAGPSGS
QQLQIPAQPASSLGADTMSAGGGGPLGDNNQGADGVGNASGDWHCDSTWMGDRVVTKS
TRTWVLPSYNNHQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLPYVVGNGTEGCLP
AFPPQVFTLPQYGYATLNRDNTENPTERSSFFCLEYFPSKMLRTGNNFEFTYNFEEVPFHSSF
APSQNLFKLANPLVDQYLYRFVSTNNTGGVQFNKNLAGRYANTYKNWFPGPMGRTQGWN
LGSGVNRASVSAFATTNRMELEGASYQVPPQPNGMTNNLQGSNTYALENTMIFNSQPANP
GTTATYLEGNMLITSESETQPVNRVAYNVGGQMLAHKFKSGDAPTTGTYNLQEIVPGSVW
MERDVYLQGPIWAKIPETGAHFHPSPAMGGFGLKHPPPMMLIKNTPVPGNITSFSDVPVSSFI
TQYSTGQVTVEMEWELKKENSKRWNPEIQYTNNYNDPQFVDFAPDSTGEYRTTRPIGTRYL
TRPL
Name: AAV2.R100 Capsid Protein
(SEQ ID NO: 3)
MAADGYLPDWLEDTLSEGIRQWWKLKPGPPPPKAAERHKDDSRGLVLPGYKYLGP
FNGLDKGEPVNEADAAALEHDKAYDRQLDSGDNPYLKYNHADAEFQERLKEDTSFGGNL
GRAVFQAKKRVLEPLGLVEEPVKTAPGKKRPVEHSPVEPDSSSGTGKAGQQPARKRLNFGQ
TGDADSVPDPQPLGQPPAAPSGLGTNTMATGSGAPMADNNEGADGVGNSSGNWHCDSTW
MGDRVITTSTRTWALPTYNNHLYKQISSQSGASNDNHYFGYSTPWGYFDFNRFHCHFSPRD
WQRLINNNWGFRPKRLNFKLFNIQVKEVTQNDGTTTIANNLTSTVQVFTDSEYQLPYVLGS
AHQGCLPPFPADVFMVPQYGYLTLNNGSQAVGRSSFYCLEYFPSQMLRTGNNFTFSYTFED
VPFHSSYAHSQSLDRLMNPLIDQYLYYLSRTNTPSGTTTQSRLQFSQAGASDIRDQSRNWLP
GPCYRQQRVSKTSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNLAISDQTKHARQAA
TADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTV
DTNGVYSEPRPIGTRYLTRNL

Name: AAV9.7m8 VP1 Capsid Protein Version 1 in which 7m8 (LALGETTRPA), underlined is placed before the Glutamine residue at the indicated site, where LGETTRP is core 7m8 sequence, and “LA” at the N-terminus and “A” at the C-terminus are linker sequences.

(SEQ ID NO: 4)
MAADGYLPDWLEDNLSEGIREWWALKPGAPQPKANQQHQDNARGLVLPGYKYLG
PGNGLDKGEPVNAADAAALEHDKAYDQQLKAGDNPYLKYNHADAEFQERLKEDTSFGGN
LGRAVFQAKKRLLEPLGLVEEAAKTAPGKKRPVEQSPQEPDSSAGIGKSGAQPAKKRLNFG
QTGDTESVPDPQPIGEPPAAPSGVGSLTMASGGGAPVADNNEGADGVGSSSGNWHCDSQW
LGDRVITTSTRTWALPTYNNHLYKQISNSTSGGSSNDNAYFGYSTPWGYFDFNRFHCHFSPR
DWQRLINNNWGFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTGNNFQFSYEFEN
VPFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGS
LIFGKQGTGRDNVDADKVMITNEEEIKTTNPVATESYGQVATNHQSALALGETTRPAQAQA
QTGWVQNQGILPGMVWQDRDVYLQGPIWAKIPHTDGNFHPSPLMGGFGMKHPPPQILIKN
TPVPADPPTAFNKDKLNSFITQYSTGQVSVEIEWELQKENSKRWNPEIQYTSNYYKSNNVEF
AVNTEGVYSEPRPIGTRYLTRNL

Name: AAV9.7m8 VP1 Capsid Protein Version 2 in which 7m8 (LALGETTRPA), underlined is placed after the Glutamine residue at the indicated site, where LGETTRP is core 7m8 sequence, and “LA” at the N-terminus and “A” at the C-terminus are linker sequences.

(SEQ ID NO: 5)
MAADGYLPDWLEDNLSEGIREWWALKPGAPQPKANQQHQDNARGLVLPGYKYLG
PGNGLDKGEPVNAADAAALEHDKAYDQQLKAGDNPYLKYNHADAEFQERLKEDTSFGGN
LGRAVFQAKKRLLEPLGLVEEAAKTAPGKKRPVEQSPQEPDSSAGIGKSGAQPAKKRLNFG
QTGDTESVPDPQPIGEPPAAPSGVGSLTMASGGGAPVADNNEGADGVGSSSGNWHCDSQW
LGDRVITTSTRTWALPTYNNHLYKQISNSTSGGSSNDNAYFGYSTPWGYFDFNRFHCHFSPR
DWQRLINNNWGFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTGNNFQFSYEFEN
VPFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGS
LIFGKQGTGRDNVDADKVMITNEEEIKTTNPVATESYGQVATNHQSAQLALGETTRPAAQA
QTGWVQNQGILPGMVWQDRDVYLQGPIWAKIPHTDGNFHPSPLMGGFGMKHPPPQILIKN
TPVPADPPTAFNKDKLNSFITQYSTGQVSVEIEWELQKENSKRWNPEIQYTSNYYKSNNVEF
AVNTEGVYSEPRPIGTRYLTRNL

Thus, in certain embodiments, the AAV2 capsid variant is or is related to an AAV2.7m8 capsid, AAV9.7m8, AAV2.5T.LSV1 capsid, or AAV2.R100 capsid that comprises, consists, or consists essentially of an amino acid sequence that is at least 80, 85, 90, 95, 98, 99, or 100% identical to the sequence set forth in SEQ ID NOS:1, 2, 3, 4 or 5.

In some embodiments, the recombinant virion or virus, e.g., an AAV, comprises a polynucleotide cassette comprising a transgene sequence that encodes a gene product, e.g., a therapeutic gene product. In certain embodiments, the transgene sequence that encodes the gene product is operably linked to a promoter sequence. In certain embodiments, the polynucleotide cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the viral vectors of the invention need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAV5, AAV6, AAV7, AAV8, AAV9, AAV10. Certain AAV vectors have the wild type REP and CAP genes deleted in whole or part, but retain functional flanking ITR sequences.

In certain embodiment, recombinant viruses or virions described herein comprises a heterologous nucleic acid comprising a nucleotide sequence (i.e., a transgene) encoding a gene product, e.g., a therapeutic gene product. In some embodiments, the gene product is an interfering RNA. In some embodiments, the gene product is an aptamer. In some embodiments, the gene product is a polypeptide. In some embodiments, the gene product is a site-specific nuclease that provides for site-specific knock-down of gene function.

Where the gene product is an interfering RNA (RNAi), suitable RNAi include but are not limited to RNAi that decrease the level of an apoptotic or angiogenic factor in a cell. For example, an RNAi can be an shRNA or siRNA that reduces the level of a gene product that induces or promotes apoptosis in a cell. Genes whose gene products induce or promote apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730.

Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44:15064). See also, U.S. Pat. Nos. 6,649,596, 6,399,586, 5,661,135, 5,639,872, and 5,639,736; and 7,947,659 and 7,919,473.

Where the gene product is an aptamer, exemplary aptamers of interest include an aptamer against vascular endothelial growth factor (VEGF). See, e.g., Ng et al. (2006) Nat. Rev. Drug Discovery 5:123; and Lee et al. (2005) Proc. Natl. Acad. Sci. USA 102:18902. For example, a VEGF aptamer can comprise the nucleotide sequence 5′-cgcaaucagugaaugcuuauacauccg-3′ (SEQ ID NO:17). Also suitable for use is a PDGF-specific aptamer, e.g., E10030; see, e.g., Ni and Hui (2009) Ophthalmologica 223:401; and Akiyama et al. (2006) J. Cell Physiol. 207:407).

Where the gene product is a polypeptide, in certain embodiments, the polypeptide may enhance function of a retinal cell, e.g., the function of a rod or cone photoreceptor cell, a retinal ganglion cell, a Muller cell, a bipolar cell, an amacrine cell, a horizontal cell, or a retinal pigmented epithelial cell. Illustrative polypeptides include neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Flt-1 polypeptide (Lai et al. (2005) Mol. Ther. 12:659); an Fc fusion protein comprising a soluble Flt-1 polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-Xl); and the like. Suitable polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF); epidermal growth factor; rhodopsin; X-linked inhibitor of apoptosis; and Sonic hedgehog, as well as functional variants and fragments of any of these, including variants having at least 80%, at least 85%, at least 90%, or at least 95% sequence identity to any of these polypeptides, and fragments comprising at least 20%, at least 30%, at least 50%, at least 60%, at least 70%, at least 80%, or at least 90% of any of these polypeptides or variants thereof.

Suitable light-responsive opsins include, e.g., a light-responsive opsin as described in U.S. Patent Publication No. 2007/0261127 (e.g., ChR2; Chop2); U.S. Patent Publication No. 2001/0086421; U.S. Patent Publication No. 2010/0015095; and Diester et al. (2011) Nat. Neurosci. 14:387.

In certain embodiments, the gene product is an anti-angiogenic polypeptide, a vascular endothelial growth factor (VEGF)-binding protein, an anti-VEGF agent, or an opsin protein.

Vascular endothelial growth factor (herein referred to as “VEGF” or “VEGF ligand”) is a potent endothelial cell-specific mitogen that plays a key role in physiological blood vessel formation. In some cases, VEGF activity results from the binding of VEGF ligand to one or more VEGF receptors in a cell. The binding of VEGF ligand to VEGF receptor may have numerous downstream cellular and biochemical effects, including but not limited to angiogenesis in tissues. VEGF has been implicated in virtually every type of angiogenic or neovascular disorder, including those associated with cancer, ischemia, and inflammation. Additionally, VEGF has been implicated in eye diseases, including but not limited to ischemic retinopathy, intraocular neovascularization, age-related macular degeneration (AMD), wet-AMD, dry-AMD, retinal neovascularization, diabetic macular edema, diabetic retina ischemia, diabetic retinal edema, proliferative diabetic retinopathy, retinal vein occlusion, central retinal vein occlusion, branched retinal vein occlusion. Further, anti-VEGF treatments, including the compositions and methods of this disclosure as described herein, may be used in the treatment of one or more of these diseases described herein. Recent data suggests that VEGF is the principal angiogenic growth factor in the pathogenesis of the wet form of AMD.

VEGF, a 46-kDa homodimeric glycopeptide, is expressed by several different ocular cell types including but not limited to pigment epithelial cells, pericytes, vascular endothelial cells, neuroglia and ganglion cells. In some cases, VEGF is expressed in specific spatial and temporal patterns during retinal development. In some cases, the human isoforms of VEGF may include proteins of 206, 189, 183, 165, 148, 145, and 121 amino acids per monomer, however the predominant human VEGF isoforms include but are not limited to VEGF121, VEGF165, VEGF189 and VEGF206. These proteins are produced by alternative splicing of the VEGF mRNA and differ in their ability to bind to heparin and to the specific VEGF receptors or coreceptors (neuropilins). The domain encoded by exons 1-5 of the VEGF gene contains information required for the recognition of the known VEGF receptors (KDR/FLK-1 and FLT-1). This domain is present in all of the VEGF isoforms. VEGF acts via these receptors, which are high-affinity receptor tyrosine kinases, leading to endothelial cell proliferation, migration, and increased vasopermeability.

VEGF is one of the several factors involved in the complex process of angiogenesis and has a very high specificity for vascular endothelial cells. VEGF is a regulator of physiological angiogenesis during processes such as embryo genesis, skeletal growth and reproductive function, but it has also been implicated in pathological angiogenesis associated with disease such as in cancer, placental disorders and other conditions. The potential biological effects of VEGF may be mediated by specific FMS-like membrane spanning receptors, FLT-1 and FLK-l/KDR. In some cases, these naturally occurring binding partners of VEGF may affect binding of VEGF to VEGF receptors, thus modulating activation of the VEGF receptor and subsequent downstream pathways.

As related to cancer, several VEGF inhibitors, including a humanized monoclonal antibody to VEGF (rhuMab VEGF), an anti-VEGFR-2 antibody, small molecules inhibiting VEGFR-2 signal transduction and a soluble VEGF receptor have shown some therapeutic properties.

As related to intraocular neovascular diseases, such as diabetic retinopathy, retinal vein occlusions, or AMD, some VEGF antagonists have shown therapeutic effects, despite the need for frequent administration.

In certain embodiments, methods disclosed herein are used to deliver an anti-VEGF agent to the eye. In particular embodiments, the recombinant virus of the present disclosure comprises a sequence encoding an anti-VEGF protein, including, but not limited to the VEGF-binding proteins or functional fragments thereof disclosed in U.S. Pat. Nos. 5,712,380, 5,861,484 and 7,071,159 and VEGF-binding fusion proteins disclosed in U.S. Pat. No. 7,635,474. In one embodiment, an anti-VEGF agent is aflibercept or a variant or functional fragment thereof. An anti-VEGF protein may also include the sFLT-1 protein as described herein.

The recombinant viruses or plasmids of the present disclosure may comprise the sequence encoding an anti-VEGF protein, including the naturally occurring protein sFlt-1, as described in U.S. Pat. No. 5,861,484 and that sequence described by SEQ ID NO: 109 of U.S. Pat. No. 5,861,484. It also includes, but is not limited to functional fragments thereof, including sequences of sFlt-1 domain 2 or those set forth in SEQ ID NO: 121 of U.S. Pat. No. 7,635,474, as well as related constructs, such as the VEGF-binding fusion proteins disclosed in U.S. Pat. No. 7,635,474. An anti-VEGF protein may also include the sFLT-1 protein as described herein. These sequences can be expressed from DNA encoding such sequences using the genetic code, a standard technique that is understood by those skilled in the art. As can be appreciated by those with skill in the art, due to the degeneracy of the genetic code, anti-VEGF protein sequences can be readily expressed from a number of different DNA sequences.

“sFlt-1 protein” herein refers to a polypeptide sequence, or functional fragment thereof, with at least 90%, or more, homology to the naturally occurring human sFLT-1 sequence, such that the sFlt-1 protein or polypeptide binds to VEGF and/or the VEGF receptor. Homology refers to the % conservation of residues of an alignment between two sequences (e.g. a Naturally occurring human sFLT-1 protein may include any suitable variants of sFLT-1, including, but not limited to functional fragments, sequences comprising insertions, deletions, substitutions, pseudofragments, pseudogenes, splice variants or artificially optimized sequences. In some cases, “sFLT-1 protein” may be at least about 90%, 91%, 92%, 93%, 94%, 95%, 96%, 97%, 98%, 99%, 99.9%, 99.99% or 100% homologous to the naturally occurring human sFLT-1 protein sequence. In some cases, “sFLT-1 protein” may be at most about 90%, 91%, 92%, 93%, 94%, 95%, 96%, 97%, 98%, 99%, 99.9%, 99.99% or 100% homologous to the naturally occurring human sFLT-1 protein sequence. In some cases, “sFLT-1 protein” may be at least about 90%>, 91%, 92%, 93%, 94%, 95%, 96%, 97%, 98%, 99%, 99.9%, 99.99% or 100% spatially homologous to the naturally occurring human sFLT-1 protein conformation. In some cases, “sFLT-1 protein” may be at most about 90%, 91%, 92%, 93%, 94%, 95%, 96%, 97%, 98%, 99%, 99.9%, 99.99% or 100% spatially homologous to the naturally occurring human sFLT-1 protein conformation.

Further, the soluble truncated form of the VEGF receptor FLT-1, sFLT-1, is the only known endogenous specific inhibitor of VEGF. In nature, it is generated by alternative mRNA splicing and lacks the membrane-proximal immunoglobulin-like domain, the transmembrane spanning region and the intracellular tyrosine-kinase domain. Structurally, FLT-1 and sFLT-1 protein may both comprise multiple functional domains. In some variants, FLT and sFLT proteins commonly share 6 interlinked domain; 3 domains involved in dimerization of the protein and 3 domains involved in the binding of a ligand, such as VEGF.

sFLT-1 is the only known endogenous specific inhibitor of VEGF. This interaction is specific and can be competed away with 100-fold excess unlabeled VEGF. In some cases, the angiostatic activity of sFLT-1 may result from inhibition of VEGF by two mechanisms: i) sequestration of VEGF, to which it binds with high affinity, and ii) formation of inactive heterodimers with membrane-spanning isoforms of the VEGF receptors FLT-1 and FLK-l/KDR. As known in the art, in vitro binding assays have indicated that sFLT-1 binds VEGF with high affinity and may also inhibit VEGF driven proliferation of human umbilical vein endothelial cells. In animal models for cancer, sFLT-1 inhibits tumor growth. In some cases, sFLT-1 may function in a substoichiometric or dominant negative manner, as excess VEGF in the extracellular space may be prevented from binding and subsequently activating the VEGF receptor. These properties of sFLT-1 have been described in Kendall and Thomas, 1993; Proc Natl Acad Sci. 90: 10705-10709, which is incorporated herein by reference in its entirety. As is known in the art, functional fragments of sFLT-1 can be used in place of the full-length protein. More specifically, the VEGF binding domain (domain 2), or alternatively domain 2 of sFLT-1 plus domain 3 from sFLT-1, KDR, or another family member, can be used to bind and inactivate VEGF. Such functional fragments are described in Wiesmann et al., 1997; Cell, 91: 695-704, which is incorporated herein by reference in its entirety. The terms “sFLT-1” and “a functional fragment of sFLT-1” are equivalent and used here interchangeably.

Suitable gene product polypeptides that may be delivered according to the methods disclosed herein also include, e.g., retinoschisin, retinitis pigmentosa GTPase regulator (RGPR)-interacting protein-1 (see, e.g., GenBank Accession Nos. Q96KN7, Q9EPQ2, and Q9GLM3); peripherin-2 (Prph2) (see, e.g., GenBank Accession No. NP000313; peripherin; a retinal pigment epithelium-specific protein (RPE65), (see, e.g., GenBank AAC39660; and Morimura et al. (1998) Proc. Natl. Acad. Sci. USA 95:3088); CHM (choroidermia (Rab escort protein 1)), a polypeptide that, when defective or missing, causes choroideremia (see, e.g., Donnelly et al. (1994) Hum. Mol. Genet. 3:1017; and van Bokhoven et al. (1994) Hum. Mol. Genet. 3:1041); and Crumbs homolog 1 (CRB1), a polypeptide that, when defective or missing, causes Leber congenital amaurosis and retinitis pigmentosa (see, e.g., den Hollander et al. (1999) Nat. Genet. 23:217; and GenBank Accession No. CAM23328).

Suitable polypeptides also include polypeptides that, when defective or missing, lead to achromotopsia, where such polypeptides include, e.g., cone photoreceptor cGMP-gated channel subunit alpha (CNGA3) (see, e.g., GenBank Accession No. NP001289; and Booij et al. (2011) Ophthalmology 118:160-167); cone photoreceptor cGMP-gated cation channel beta-subunit (CNGB3) (see, e.g., Kohl et al. (2005) Eur J Hum Genet. 13(3):302); guanine nucleotide binding protein (G protein), alpha transducing activity polypeptide 2 (GNAT2) (ACHM4); and ACHM5; and polypeptides that, when defective or lacking, lead to various forms of color blindness (e.g., L-opsin, M-opsin, and S-opsin). See Mancuso et al. (2009) Nature 461(7265):784-787.

In some cases, a gene product of interest is a site-specific endonuclease that provide for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease can be targeted to the defective allele and knock out the defective allele.

In addition to knocking out a defective allele, a site-specific nuclease can also be used to stimulate homologous recombination with a donor DNA that encodes a functional copy of the protein encoded by the defective allele. Thus, e.g., a subject rAAV virion can be used to deliver both a site-specific endonuclease that knocks out a defective allele, and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217. In some embodiments, a subject rAAV virion comprises a heterologous nucleotide sequence that encodes a site-specific endonuclease; and a heterologous nucleotide sequence that encodes a functional copy of a defective allele, where the functional copy encodes a functional retinal protein. Functional retinal proteins include, e.g., retinoschisin, RPE65, retinitis pigmentosa GTPase regulator (RGPR)-interacting protein-1, peripherin, peripherin-2, and the like.

Site-specific endonucleases that are suitable for use include, e.g., zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073.

In some embodiments, a nucleotide sequence encoding a gene product is operably linked to a constitutive promoter. In other embodiments, a nucleotide sequence encoding a gene product of interest is operably linked to an inducible promoter. In some instances, a nucleotide sequence encoding a gene product of interest is operably linked to a tissue-specific or cell type-specific regulatory element. In certain embodiments, the promoter selected from cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter.

For example, in some instances, a nucleotide sequence encoding a gene product of interest is operably linked to a photoreceptor-specific regulatory element (e.g., a photoreceptor-specific promoter), e.g., a regulatory element that confers selective expression of the operably linked gene in a photoreceptor cell. Suitable photoreceptor-specific regulatory elements include, e.g., a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225).

Recombinant viral vectors (e.g., rAAV virions) described herein, and optionally encapsulating polynucleotide cassettes of the present disclosure, may be produced using standard methodology. For example, in the case of rAAV virions, an AAV expression vector comprising a polynucleotide cassette may be introduced into a producer cell, followed by introduction of an AAV helper construct comprising a polynucleotide sequence encoding a variant capsid protein disclosed herein, and where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions comprising variant capsid proteins described herein are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety.

### Methods

The present invention also provides methods of delivering a gene product to a cell or tissue, comprising administering a virus or viral vector described herein to the cell or tissue. Certain embodiments of the present invention relate to methods of transducing AAV vectors followed by expression of a transgene in the presence of activated complement cascade in a subject comprising administering to the subject a recombinant adeno-associated virus (rAAV) virion at an amount capable of at least partially blocking C3 in the subject.

In particular embodiments, the rAAV may be any of those described herein. In some instances, the rAAV can be a native AAV of serotype 1, 2, 3, 4, 5, 6, 7, 8, or 9. In some instances, the rAAV can be a chimeric AAV comprising proteins from at least two serotypes. In some cases, the rAAV can comprise a variant capsid protein. In some instances, the rAAV can comprise amino acid insertions, deletions, or substitutions relative to the sequence of a parent natural AAV. In some embodiments, the rAAV can comprise an amino acid insertion in a GH loop of a capsid protein. Examples of such insertions are described in U.S. Pat. Nos. 9,193,956 and 8,663,624, the disclosure of each are incorporated by reference herein in their entirety.

Described herein is development of novel AAV vectors for clinical indications with IVT delivery that can overcome current limitations. AAV2.7m8 (described in U.S. Pat. No. 9,193,956, the disclosure of which is incorporated by reference herein in its entirety) is a novel variant of AAV2 that can effectively transduce multiple layers of the non-human primate (NHP) retina following IVT injection.

The present invention provides new methods of administration of viral vectors, e.g., AAV vectors, that are demonstrated herein to successfully transduce ocular cells in the presence of an activated complement cascade. In particular embodiments of any of the methods described herein, the administration is performed by ocular administration, retinal administration, subretinal administration and/or intravitreal administration. In particular embodiments, the administration is performed by ocular injection, retinal injection, subretinal injection and/or intravitreal injection. In certain embodiments, the subject being administered the viral vector has activated complement cascade. For example, a subject may have been previously administered rAAV by an IVT injection, which provoked activation of complement cascade.

In one embodiment, the disclosure includes a method of transducing AAV for expression of a transgene in the presence of activated complement cascade in a subject comprising administering to the subject a recombinant adeno-associated virus (rAAV) virion at an amount capable of at least partially blocking C3 in the subject. In particular embodiments, the rAAV virion is AAV2.7m8. In particular embodiments, the subject is a mammalian subject. In various embodiments, the rAAV virion administered is at least about 1×10⁹ vg, at least about 1×10¹¹ vg, at least about 5×10¹¹ vg, at least about 1×10¹³ vg, at least about 5×10¹³ vg, at least about 1×10¹⁴ vg, at least about 5×10¹⁴ vg, at least about 1×10¹⁵ vg, at least about 5×10¹⁵ vg, or at least about 1×10¹⁶ vg. In particular embodiments, the administration is performed by ocular administration, retinal administration, subretinal administration and/or intravitreal administration. In particular embodiments, the administration is performed by ocular injection, retinal injection, subretinal injection and/or intravitreal injection. In particular embodiments, the administration is an intravitreal injection. In certain embodiments, the subject is administered a first dose and a second dose of the rAAV virion, each administered dose comprising an amount of rAAV virion capable of at least partially blocking C3 in the subject.

In one embodiment, the disclosure includes a method of delivering a transgene encoding a gene product to an ocular cell in a subject, comprising administering to one or more sites within the eye of the subject an effective amount of a rAAV comprising the transgene. In certain embodiments, the transgene is expressed in the presence of activated complement cascade in the subject, and the effective amount of the rAAV administered is sufficient to at least partially block C3 in the subject and transduce the ocular cell, wherein the rAAV-transduced ocular cell expresses the gene product. In particular embodiments, the rAAV virion is AAV2.7m8. In particular embodiments, the subject is a mammalian subject. In particular embodiments, the administration is performed by ocular administration, retinal administration, subretinal administration and/or intravitreal administration. In particular embodiments, the administration is performed by ocular injection, retinal injection, subretinal injection and/or intravitreal injection. In particular embodiments, the administration is an IVT injection.

In various embodiments of the methods described herein, the effective amount of the rAAV administered is at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, at least about 1×10¹⁴ vg, at least about 5×10¹⁴ vg, at least about 1×10¹⁵ vg, at least about 5×10¹⁵ vg, or at least about 1×10¹⁶ vg. In some embodiments, the effective amount of the rAAV administered is about 1×10¹¹ vg to about 5×10¹² vg, or about 1×10¹¹ vg to about 1×10¹⁴ vg.

In certain embodiments, the rAAV is administered retinally, subretinally, and/or intravitreally.

In certain embodiments, the gene product delivered by methods of the present invention may be any therapeutic gene product, including but not limited to any gene products disclosed herein. In some embodiments of any of the methods described here, the gene product is an anti-angiogenic polypeptide. In other embodiments, the gene product is a vascular endothelial growth factor (VEGF)-binding protein. In certain embodiments, the gene product is an anti-VEGF agent or anti-VEGF protein. In certain embodiments, the gene product is an opsin protein. In certain embodiments, the gene product is anti-C3 protein.

In certain embodiments, methods disclosed herein are used to provide multiple administrations of a viral vector, e.g., rAAV, to a subject, e.g., by an IVT injection. As demonstrated herein, the present methods are able to successfully transduce ocular cells by an IVT injection in the presence of activated complement cascade. Accordingly, the methods may be employed to successfully deliver a gene product by an IVT injection to ocular cells within a subject who has such activated complement cascade.

In one embodiment, the disclosure includes a method for treating an ocular disease or disorder, such as geographic atrophy associated with dry AMD in a subject comprising: first, administering to a first eye of the subject a first effective amount of a first viral vector (e.g., rAAV) comprising a first transgene encoding a first gene product (i.e., a first dose), next, waiting for a period of time, and then, administering to a second eye of the subject a second effective amount of a second viral vector (e.g., rAAV) comprising a second transgene encoding a second gene product (i.e., a second dose), wherein the first and second effective amounts of viral vector (e.g., rAAV) are amounts sufficient to transduce cells of the eye, wherein the transduced cells express the first and second gene products. In certain embodiments, the method may further comprise subsequent administrations of a viral vector following additional periods of time. In particular embodiments, the methods may comprise two or more, three or more, four or more, or five or more administrations of a viral vector with periods of time between each administration. In particular embodiments, the administration is performed by ocular administration, retinal administration, subretinal administration and/or intravitreal administration. In particular embodiments, each administration is independently performed by ocular injection, retinal injection, subretinal injection and/or intravitreal injection. In particular embodiments, the administration is an intravitreal injection.

In specific embodiments, the first viral vector (e.g., rAAV) and the second viral vector (e.g., rAAV) are of the same serotype. In other embodiments, the first and second viral vectors (e.g., rAAVs) comprise the same capsid proteins. In certain embodiments, the first rAAV and the second rAAV are both AAV, e.g., AAV2.7m8. In some embodiments, the first and second viral vectors (e.g., rAAVs) are different serotypes.

In certain embodiments, the period of time between administering the first viral vector (e.g., rAAV) and administering the second viral vector (e.g., rAAV) is at least about one week, at least about one month, at least about three months, at least about six months, at least about one year, at least about 18 months, at least about three years, or longer than about three years. In some embodiments, the subject is not administered a rAAV during the period of time between administering the first rAAV and administering the second rAAV. In certain embodiments, the subject may receive one, two, three, four, or more effective amounts of rAAV in either one or both eyes, following the second administration of rAAV. In other embodiments, the subject does not receive any additional administrations of rAAV after the second administration of rAAV.

In certain embodiments of any of the methods described herein that comprise administering a first dose (i.e., a first effective amount) and a second dose (i.e., a second effective amount) of the rAAV, the first dose and second dose are administered to the same eye. In certain embodiments, the first dose and second dose are administered to different eyes. In particular embodiments, the first and second dose are administered for treating the same ocular disease. In certain embodiments, the first and second dose are administered for treating different ocular diseases. In certain embodiments, the first dose and second dose comprise rAAV comprising the same heterologous polynucleotide encoding a gene product. In particular embodiments, the first dose and second dose comprise rAAV comprising different heterologous polynucleotides, e.g., encoding different gene products.

In one embodiment, the present disclosure includes a method of treating an ocular disease or disorder, e.g., geographic atrophy associated with dry AMD, in a subject in need thereof, comprising the following:
(1) administering to one or both eyes of the subject by ocular injection (e.g., retinal, subretinal and/or intravitreal injection) an effective amount of a rAAV comprising a transgene encoding a therapeutic gene product, wherein at least a plurality of ocular cells (e.g., retinal cells) of the one or both eyes are transduced and express the therapeutic gene product;
(2) waiting for a period of time; and
(3) administering to one or both eyes of the subject by ocular injection (e.g., retinal, subretinal and/or intravitreal injection) an effective amount of a rAAV comprising a transgene encoding a therapeutic gene product, wherein at least a plurality of ocular cells (e.g., retinal cells) of the one or both eyes are transduced and express the therapeutic gene product. In certain embodiments, step (1) and/or step (3) are performed by an IVT injection.

In certain embodiments, the therapeutic gene product administered at step (1) and/or step (3) is an anti-VEGF agent or protein, e.g., sFlt-1 or aflibercept. In certain embodiments, the therapeutic gene product administered at step (1) is the same as the therapeutic gene product administered at step (3). In other embodiments, the first or second therapeutic gene product may be an anti-C3 antibody. In other embodiments, the anti-C3 protein such as an anti-C3 antibody may be administered only once.

In other embodiments, the methods are practiced to deliver any of the gene products disclosed herein.

In particular embodiments, the rAAV administered in step (1) and/or step (3) is rAAV2.7m8.

In certain embodiments, the time period of step (b) is at least one week, at least one month, at least three months, at least six months, at least one year, at least 18 months, at least two years, at least three years, or longer than three years.

In particular embodiments, the effective amount administered in step (1) is at least about 1×10⁹ vg, at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, or at least about 1×10¹⁴ vg. at least about 5×10¹⁵ vg, or at least about 1×10¹⁶ vg. In some embodiments, the effective amount of the rAAV administered in the first effective dose is about 1×10⁹ vg up to about 5×10¹² vg, or about 1×10¹¹ vg up to about 1×10¹⁴ vg.

In particular embodiments, the effective amount administered in step (3) is at least about 1×10⁹ vg, at least about 1×10¹¹ vg, at least about 5×10¹¹ vg, at least about 1×10¹² vg, at least about 5×10¹² vg, at least about 1×10¹³ vg, at least about 5×10¹³ vg, at least about 1×10¹⁴ vg, at least about 5×10¹⁵ vg, at least about 1×10¹⁵ vg, at least about 5×10¹⁵ vg, or at least about 1×10¹⁶ vg. In certain embodiments, the effective amount of the rAAV administered in the second effective dose is at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, or at least about 1×10¹⁴ vg. In some embodiments, the effective amount of the rAAV administered in the second effective dose is about 1×10¹¹ vg up to about 5×10¹² vg, or about 1×10¹¹ vg up to about 1×10¹⁴ vg.

In certain embodiments of any of the methods described herein, the therapeutic gene product is an anti-VEGF agent or protein.

In certain embodiments of any of the methods disclosed herein, the disease or disorder being treated is associated with aberrant angiogenesis in the eye. In particular embodiments, the disease or disorder is macular degeneration, e.g., age-related macular degeneration (AMD). In particular embodiments, it is wet macular degeneration or dry macular degeneration.

In some embodiments of any of the methods described herein, the subject is not administered an immunosuppressant prior to, concurrent with, or following the administration of the first rAAV. In other embodiments, the subject is administered an immunosuppressant prior to, concurrent with, or following administration of the first rAAV. In specific embodiments, the subject is not administered an immunosuppressant after the administration of the first rAAV. In certain embodiments, an immunosuppressant is administered to the subject after the administration of the first rAAV and before or concurrent with the administration of the second rAAV. In some embodiments, and immunosuppressant is administered is administered to the subject after the administration of the second rAAV.

In some embodiments of any of the methods described here that comprise administering a first dose and a second dose of the rAAV, the administering of the first effective amount and the administering of the second effective amount is by intraocular injection or intravitreal injection. In other embodiments, the administration of the second effective amount of the second rAAV provides a therapeutic effect in the eye of the subject. In certain embodiments, the administration of the first effective amount and the second effective amount are to the same eye of the subject. In other embodiments, the administration of the first effective amount and the second affective amount are to different eyes of the subject. In specific embodiments, the first effective amount and the second effective amount are treating the same ocular disease or disorder. In some embodiments, the first effective amount and the second effective amount are treating different ocular diseases or disorders. In certain embodiments, the first gene product and the second gene product are the same. In other embodiments, the first gene product and the second gene product are different.

In certain embodiments of any of the methods disclosed herein for treating an ocular disease or disorder, the first effective amount of viral vector and the second effective amount of viral vectors are amounts sufficient to transduce at least a plurality of ocular cells, resulting in the expression of a therapeutically effective amount of the gene product, i.e., an amount sufficient to treat the disease or disorder.

Any of the viruses or viral vectors described herein may present in a pharmaceutical composition and/or administered to a subject while in a pharmaceutical composition. A pharmaceutical composition is a formulation containing one or more active ingredients as well as one or more excipients, carriers, stabilizers or bulking agents, which is suitable for administration to a human patient to achieve a desired diagnostic result or therapeutic or prophylactic effect. For storage stability and convenience of handling, a pharmaceutical composition can be formulated as a lyophilized (i.e. freeze-dried) or vacuum dried powder which can be reconstituted with a buffer prior to administration to a patient.

The vector or recombinant viruses (virions) can be incorporated into pharmaceutical compositions for administration to mammalian patients, particularly humans. The vector or virions can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the therapeutic molecule dissolved in an aqueous buffer having an acceptable pH upon reconstitution.

In some aspects, the pharmaceutical composition provided herein comprise a therapeutically effective amount of a vector or virion in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some aspects, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the anti-VEGF contained in the adenoviral vector delivery system, may be in the range of 6.5 to 7.75, 7 to 7.5, or 7.2 to 7.4. The pH of the formulation may range from about 3.0 to about 12.0. The pH of the immunogenic composition may be at least about 3, 4, 5, 6, 7, 8, 9, 10, 11 or 12 pH units. The pH of the immunogenic composition may be at most about 3, 4, 5, 6, 7, 8, 9, 10, 11 or 12 pH units.

The present invention further provides unit dosage forms of a viral vector or virus described herein. In particular embodiments, an amount the virus or viral vector may be measured as vector genomes. In some cases, the unit dose of the pharmaceutical composition of the disclosure is 1×10¹⁰ to 3×10¹² vector genomes. In some cases, the unit dose of the pharmaceutical composition of the disclosure is 1×10⁹ to 3×10¹³ vector genomes. In some cases, the unit dose of the pharmaceutical composition of the disclosure is 1×10¹⁰ to 1×10¹¹ vector genomes. In some cases, the unit dose of the pharmaceutical composition of the disclosure is 1×10⁸ to 3×10¹⁴ vector genomes. In some cases, the unit dose of the pharmaceutical composition of the disclosure is at least about 1×10¹, 1×10², 1×10³, 1×10⁴, 1×10⁵, 1×10⁶, 1×10⁷, 1×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰ 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1×10¹³, 1×10¹⁴, 1×10¹⁵, 1×10¹⁶, 1×10¹⁷, and 1×10¹⁸ vector genomes. In some cases, the unit dose of the pharmaceutical composition of the disclosure is 1×10⁸ to 3×10¹⁴ vector genomes. In some cases, the unit dose of the pharmaceutical composition of the disclosure is at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some embodiments the unit dose of the pharmaceutical composition comprises at least about 2×10⁹ vg, at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, or at least about 1×10¹⁴ vg. In some embodiments, the unit dose is about 1×10¹¹ vg up to about 5×10¹² vg, or about 1×10¹¹ vg up to about 1×10¹⁴ vg. In certain embodiments, the unit dose is at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, or at least about 1×10¹⁴ vg. In some embodiments, the unit dose is about 1×10¹¹ vg up to about 5×10¹² vg, or about 1×10¹¹ vg up to about 1×10¹⁴ vg. In particular embodiments, the unit dose is at least about 1×10¹⁰ vg, at least about 1×10¹¹ vg, at least about 5×10¹¹ vg, at least about 1×10¹² vg, at least about 5×10¹² vg, or at least about 1×10¹³ vg. In some embodiments, the unit dose is about 1×10¹¹ vg to about 5×10¹² vg, or about 1×10¹¹ vg to about 1×10¹³ vg. In particular embodiments, the unit dose is at least about 5×10¹² vg, at least about 1×10¹³ vg, at least about 5×10¹³ vg, or at least about 1×10¹⁴ vg, at least about 5×10¹⁴ vg, or at least about 1×10¹⁵ vg. In some embodiments, the unit dose is about 1×10¹² vg to about 5×10¹⁴ vg, or about 5×10¹² vg to about 1×10¹⁴ vg.

In some aspects, following a method of treatment disclosed herein, a subject's best corrected visual acuity (BCVA) improves by 1, 2 3, 4, 5 or more lines.

In some aspects, following a method of treatment disclosed herein, a reduction in neovascularization as assessed by Fluorescein angiography (FA) occurs.

In some cases, retinal thickness may be measured to examine the effects of treatment. In some cases, following a method of treatment disclosed herein, the central retinal thickness of the human subject does not increase by more than 50 microns, 100 microns, or 250 microns within 12 months following treatment with the pharmaceutical composition of the disclosure. In some cases, following a method of treatment disclosed herein, the central retinal thickness of the human subject decreases by at least 50 microns, 100 microns, 200 microns, 250 microns, 300 microns, 400 microns, 500 microns, 600 microns within 3 months, 6 months or 9 months 12 months following treatment with the pharmaceutical composition of the disclosure. The decrease in the central retinal thickness of the human subject may be measured comparing the central retinal thickness at point in time to a baseline measurement taken at or within 1, 3, 7 or 10 days of the administration of the pharmaceutical composition of the disclosure.

Compositions and reagents useful for the present disclosure may be packaged in kits to facilitate application of the present disclosure. In some aspects, the present method provides for a kit comprising a virus or viral vector disclosed herein, e.g., a recombinant virus of the disclosure. In some embodiments, the kit comprises instructions for using the virus or viral vector, e.g., instructions on how to administer the virus or viral vector to a subject to treat an ocular disease or disorder. The instructions could be in any desired form, including but not limited to, printed on a kit insert, printed on one or more containers, as well as electronically stored instructions provided on an electronic storage medium, such as a computer readable storage medium. Also optionally included is a software package on a computer readable storage medium that permits the user to integrate the information and calculate a control dose.

In one aspect, a kit comprises one or more containers, each container comprising: (a) a unit dose of a pharmaceutical composition described herein, which comprises a recombinant virus provided herein (e.g., a rAAV), and (b) instructions on how to administer to a subject a therapeutically effective amount of the recombinant virus.

In some embodiments, the kit comprises two containers, wherein each container comprises a unit dose of a pharmaceutical composition described herein. In certain embodiments, the unit dose in each of the two containers is the same, whereas in other embodiments, the unit dose in each of the two containers is different. In particular embodiments, both of the two containers comprises a unit dose of at least about 1×10¹⁰ vg, at least about 1×10¹¹ vg, at least about 5×10¹¹ vg, at least about 1×10¹² vg, at least about 5×10¹² vg, at least about 1×10¹³ vg, at least about 5×10¹³ vg, or at least about 1×10¹⁴ vg. In some embodiments, one container for the first dose comprises about 1×10¹¹ vg up to about 5×10¹² vg, or either about 1×10¹⁰ vg up to about 1×10¹⁴ vg or about 1×10¹¹ vg up to about 1×10¹⁴ vg. In some embodiments, the second container for the second dose comprises about 1×10¹¹ vg up to about 5×10¹² vg, or either about 1×10¹¹ vg up to about 1×10¹⁴ vg or about 1×10¹¹ vg up to about 1×10¹⁴ vg. In some embodiments, the first container comprises at least about 1×10¹⁰ vg, at least about 1×10¹¹ vg, at least about 5×10¹¹ vg, at least about 1×10¹² vg, at least about 5×10¹² vg, or at least about 1×10¹³ vg. In some embodiments, the second container comprises at least about 5×10¹² vg, at least about 1×10¹³ vg, at least about 5×10¹³ vg, at least about 1×10¹⁴ vg, at least about 5×10¹⁴ vg, or at least about 1×10¹⁵ vg. In some embodiments, any unit dose comprises about 1×10¹² vg to about 5×10¹⁴ vg, or about 5×10¹² vg to about 1×10¹⁵ vg.

In some embodiments, the unit dose of rAAV particles is administered in combination with steroid treatment. In some embodiments, the steroid treatment is a corticosteroid treatment (e.g. dexamethasone SP, or triamcinolone). In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the ophthalmic steroid treatment is a glucocorticoid including, but not limited to, an anti-inflammatory glucocorticoid. In some embodiments, the topical steroid treatment is a glucocorticoid including but not limited to, an anti-inflammatory glucocorticoid. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. Anti-inflammatory glucocorticoids may include, but are not limited to, difluprednate, dexamethasone, prednisolone, triamcinolone, fluorometholone, rimexolone, fluocinolone, loteprednol and bioequivalents thereof. In some embodiments, the topical steroid treatment is a difluprednate treatment. By “dexamethasone” is intended dexamethasone, dexamethasone biosimilars, dexamethasone bioequivalents, and pharmaceutical compositions comprising dexamethasone, a dexamethasone biosimilar or a dexamethasone bioequivalent. Pharmaceutical compositions comprising dexamethasone include, but are not limited to, Ozurdex™, Maxidex™, Decadron™ Dexamethasone Intensol™, Ocu-Dex™, Dexycu™, Dextenza™ and Zodex™. Ozurdex™ is a pharmaceutical composition comprising dexamethasone. By “difluprednate” is intended difluprednate, difluprednate biosimilars, difluprednate bioequivalents, and pharmaceutical compositions comprising difluprednate, a difluprednate biosimilar or a difluprednate bioequivalent. Pharmaceutical compositions comprising difluprednate include, but are not limited to, Durezol™ and difluprednate emulsions. By “triamcinolone” is intended triamcinolone, triamcinolone biosimilars, triamcinolone bioequivalents, and pharmaceutical compositions comprising triamcinolone, a triamcinolone biosimilar or a triamcinolone bioequivalent. Pharmaceutical compositions comprising triamcinolone include, but are not limited to, Triesence™, Xipere™, and Trivaris™. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment (e.g., difluprednate). In some embodiments, the ophthalmic steroid treatment (e.g., difluprednate) is a daily steroid treatment for up to about 4 weeks, about 6 weeks, or about 8 weeks from administering the unit dose of rAAV particles. In some embodiments, the ophthalmic steroid treatment comprises about four administrations of ophthalmic steroid on about week 1, about three administrations of ophthalmic steroid on about week 2, about two administrations of ophthalmic steroid on about week 3, and about one administration of ophthalmic steroid on about week 4; timing starting with and following administration of the unit dose of rAAV particles. In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the steroid treatment is an ophthalmic steroid treatment (e.g., difluprednate). In some embodiments, the ophthalmic steroid treatment (e.g., difluprednate) is a daily topical steroid treatment for up to about 4 weeks, about 6 weeks, or about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid (i.e., QID) per day for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the topical steroid comprises difluprednate 0.05% at a dose of about 1 μg to about 3 μg. In some embodiments, the topical steroid comprises difluprednate 0.05% at a dose of about 2.5 μg. In some embodiments, the topical steroid is about 0.005% to about 0.5% difluprednate. In some embodiments, the topical steroid is any of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% difluprednate. In some embodiments, the topical steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some aspects, the kit may comprise pharmaceutically acceptable salts or solutions for administering the recombinant virus. Optionally, the kit can further comprise instructions for suitable operational parameters in the form of a label or a separate insert. For example, the kit may have standard instructions informing a physician or laboratory technician to prepare a dose of recombinant virus.

Optionally, the kit could further comprise devices for administration, such as a syringe, filter needle, extension tubing, cannula, and subretinal injector.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1—Designs of Cassettes

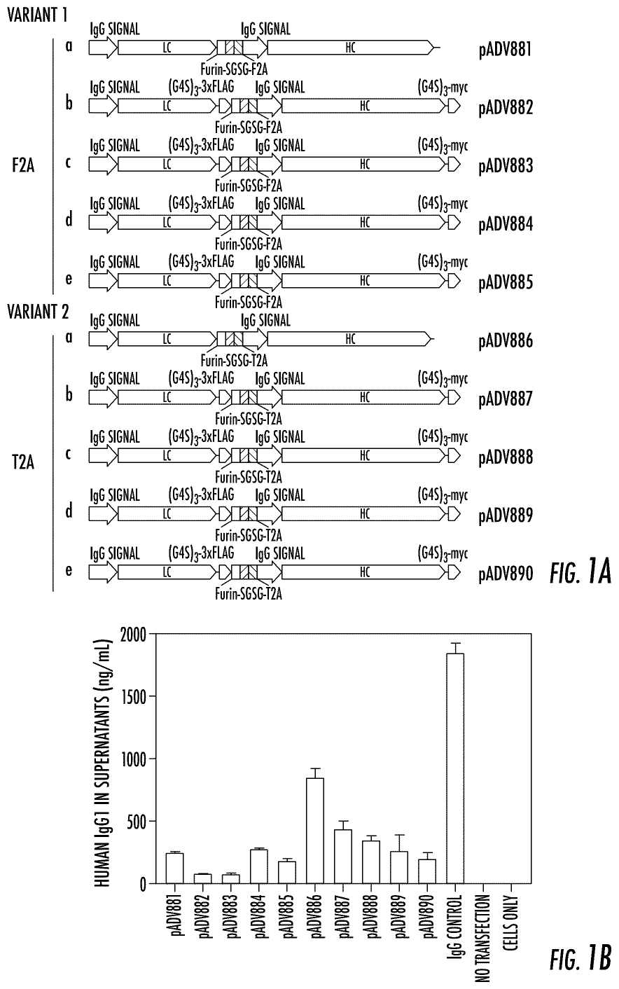

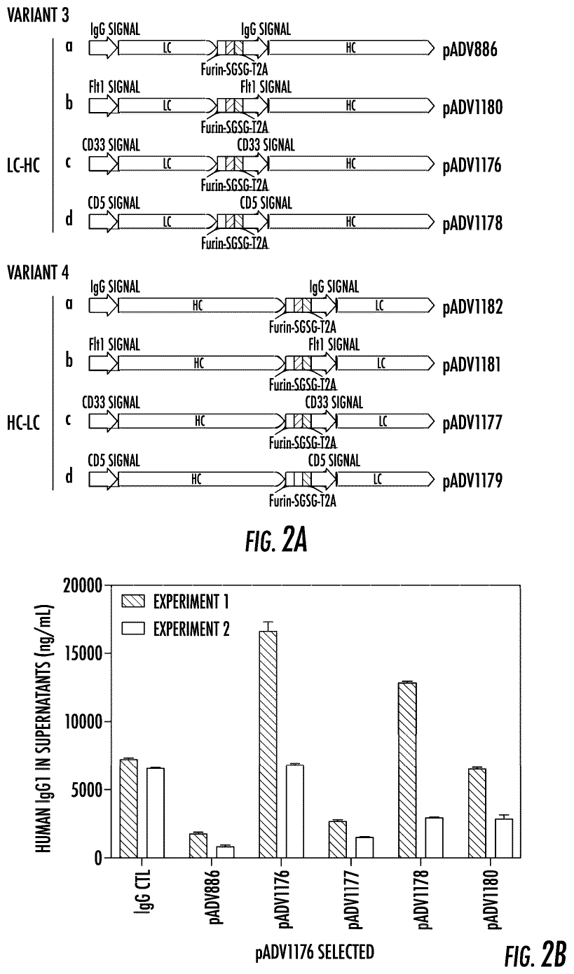

The first-generation screen included ten cassettes. In all cassettes, the light chain (LC) and heavy chain (HC) are preceded by a Kappa signal sequence at the N-terminus. Except for the pADV881 and pADV886 cassettes, all cassettes contained a (G4S)₃-myc tag sequence at the C-terminus of the LC and HC region. The LC and HC complexes of each cassette are bridged with a linker sequence (Furin-SGSG-F2A, FIG. 1A Variants 1 or Furin-SGSG-T2A, FIG. 1A Variants 2). The 10 plasmids named as pADV881 to pADV890 were synthesized by ATUM (Newark, CA, USA). Suspension HEK293 cells (Oxford Genetics) were transfected with the 10 plasmids (cell culture and transfection procedure can be found in following section). Human IgG1 in supernatant from transfected cells was measured by human IgG1 ELISA according to manufacturer's protocol (Cayman Chemical, Cat #: 500910). Based on the robust expression of human IgG1 in supernatants (FIG. 1), candidate pADV886 (T2A linker, untagged, no CpG optimization) was selected as the template for the second-generation construct design. Second generation variables included: different signal sequences and orientation of the tandem LC and HC region (FIG. 2A). Seven plasmids synthesized by ATUM (Newark, CA, USA) were transfected into suspension HEK293 cells (cell culture and transfection procedure can be found in following section). Human IgG1 expression in supernatant was measured by using human IgG1 ELISA kit (Cayman Chemical, Cat #500910) and confirmed pADV1176 as a lead candidate for further investigation (FIG. 2B). The nucleotide sequences of the seventeen cassettes discussed above are set forth in SEQ ID NOS:6-16 and 18-24; amino acid sequences of a few of the preferred signal peptides are set forth in SEQ ID NOS:26-29. Also included below are sequences of the light chain (SEQ ID NO:25) and heavy chain sequences (SEQ ID NO:24) of anti-C3 antibody.

### Example 2—Quantify Human IgG1 Expression Using ELISA and Western Blot

Suspension HEK293 cells (Oxford Genetics) were cultured on 6-well plates (3 mL media per well; culture media include BalanCD Media (Irvine Scientific, Cat #: 91165) and 4 mM L-Glutamine (Gibco, Cat #: 25030)) at 37° C. (95% O2 and 5% CO2). These cells were individually transfected with plasmids using TransIT-LT1 transfection reagent (MIR2305, Mirus Bio) according to manufacturer's protocol. Briefly, 2×10⁶ cells/mL were transfected with 1 μg of DNA per mL of cells. Seventy two hours post-transfection, cells and media were collected from wells and centrifuged at 3,200 rpm for 10 minutes at 4° C. Supernatants were collected for further analysis. Human IgG1 levels in the supernatant was measured by human IgG1 ELISA according to manufacturer's protocol (Cayman Chemical, Cat #: 500910) (FIG. 1B and FIG. 2B). Plate was read on a SpectraMax M3 Plate Reader (Molecular Devices, CA, USA).

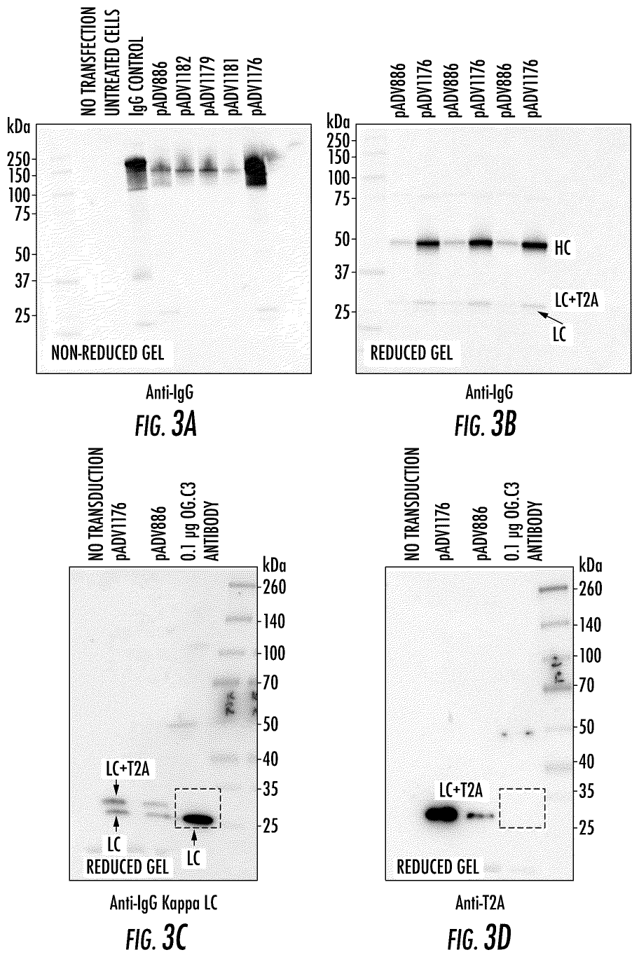

Full length and light chain/heavy chain (LC/HC) of the anti-C3 antibodies were analyzed by Western Blot. Equal volumes of cell supernatant (20 μL/lane) of HEK293 or rabbit retinal explants were incubated with reducing (Thermo Fisher Scientific, Cat #: 39000; FIGS. 3C and 3D) or non-reducing buffer (Thermo Fisher Scientific, Cat #: 39001, FIG. 3A) at 90° C. for 10 minutes before being loaded on 4-12% Bis-Tris gels (Thermo Fisher Scientific, Cat #: NP0326BOX). Electrophoresis was performed at 125 volts for 60 minutes. Proteins were transferred (6 minutes at 23 volts) to an iBlot 2 PVDF Mini Stack (Thermo Fisher Scientific, Cat #: IB24002) using an iBlot 2 Western Blotting System (Thermo Fisher Scientific). Blots in FIGS. 3A and 3B were probed with a peroxidase affiniPure goat-anti-human secondary antibody (Jackson ImmunoResearch, Cat #: 109-035-003) at room temperature for 1 hour. Blots in FIG. 3C were detected by a goat anti-human kappa light chain antibody (Invitrogen, Cat #: SA510285, 1:2000,) overnight night at 4° C. Blots in FIG. 3D were stained by a mouse anti-T2A antibody (Novus, Cat #: T2A NPB2-59627, 1:2000,) overnight night at 4° C. The positive control (FIGS. 3C-3D) was OG.C3 antibody (LakePharma, Cat #: SR-20166). The next day, the blots were probed with secondary antibodies for 1 hour at room temperature. The blot was subjected to ECL Western Blotting Substrate (Thermo Fisher Scientific, Cat #: 32106). The bands of full length (FIG. 3A), the LC/HC (FIG. 3B), LC (FIG. 3C) and T2A (FIG. 3D) were imaged with an Amersham ImageQuant 800 (Cytiva). Detailed information on the supernatant collection from rabbit retinal explants can be found below herein.

### Example 3—Preparation of Viral Vectors and Transduction Potency

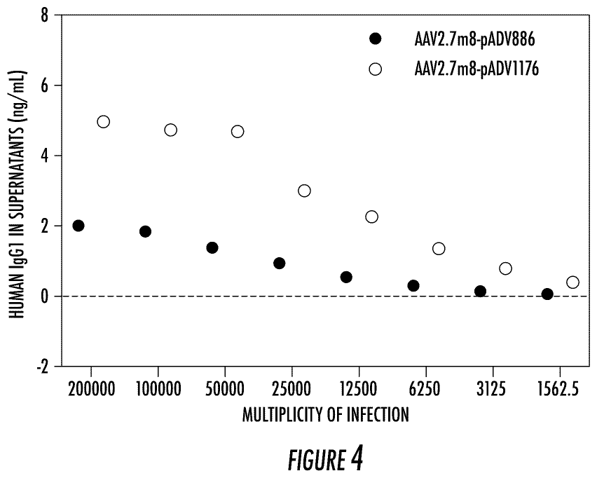

Two candidates (pADV886 and pADV1176) were vectorized using AAV2.7m8 capsids in-house, at ADVERUM Biotechnology, Inc (Redwood City, CA). Transduction potency of both vectors were evaluated using human retinal pigment epithelial (ARPE19) cells (ATCC, Cat #: CRL-2302, Lot: 70022669, Passage 10). Briefly, ARPE19 cells were seeded at 10,000 cells/well in a 96-well plate. On the following day, cells were transduced with AAV2.7m8-pADV886 or AAV2.7m8-pADV1176 at varying multiplicity of infections (MOIs); 200000, 100000, 50000, 25000, 12500, 6250, 3125 and 1562.5. Cell culture supernatant was harvested 72 hours post-transduction and immediately assayed using a human IgG1 ELISA kit (Invitrogen, Cat #: EHIGG1). The concentration of human IgG1 detected in cell culture supernatant correlates to vector-based antibody expression (FIG. 4).

### Example 4—Validation of the Viral Vector Expression Using Rabbit Retinal Explants

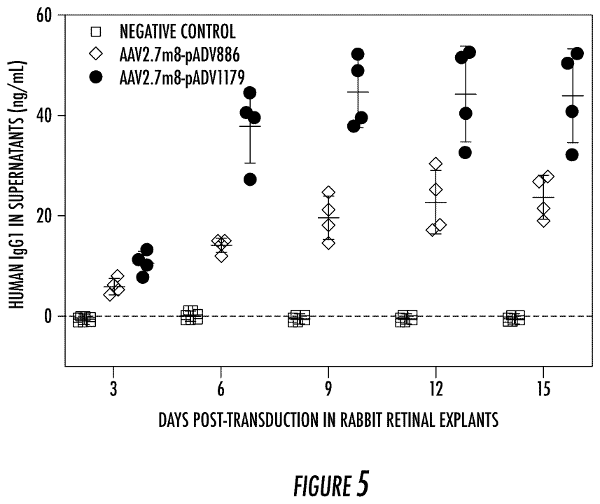

Retinas were carefully dissected from adult rabbit eyes 2-3 hours post-euthanasia. Six mm retina biopsy punches were obtained and mounted onto transwell inserts of cell culture dishes (Corning, Cat #: CLS3401-48EA), with the photoreceptor side facing downwards and the ganglion cell side facing upwards. The explants were incubated with complete media (Formulation in Table 1) for 24 hours in a 37° C. cell culture incubator (95% O₂ and 5% CO₂). The next day, media was completely removed, and the explants were transduced with AAV2.7m8-pADV886 or AAV2.7m8-pADV1176 at 2×10¹⁰ vg/explant diluted in 100 μL of transduction media (Formulation in Table 2). Explants were incubated for 2 hours in a cell culture incubator. Afterwards, 400 μL of complete media was added to each well (in between the inserts and well wall). Supernatant was collected at days 3, 6, 9, 12 and 15 post-transduction and replaced with fresh complete media. The collected supernatant was immediately stored at −80° C. until the experiment was completed. Samples were assayed with a human IgG1 ELISA kit (Invitrogen, Cat #: EHIGG1) in which samples were diluted 1:4 using kit assay diluent. The analysis indicates that AAV2.7m8-pADV1176 has more robust secreted levels compared to AAV2.7m8-pADV886 (FIG. 5).

### Example 5—Validation of Viral Vector Expression by Intravitreal Injection

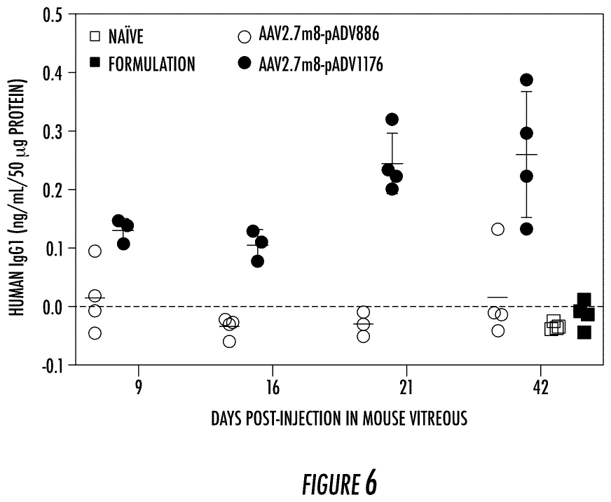

Intravitreal injections of AAV2.7m8-pADV886 or AAV2.7m8-pADV1176 were performed (˜1×10⁹ vg/eye) in C57BL/6 adult mice according to the protocol set-up in Table 3. Retinas were collected at four timepoints, days 9, 16, 21 and 42 post-injection, in which the retinas were dissected on ice and snap-frozen on dry ice prior to storage at −80° C. To determine the levels of human IgG1 expressed in mouse retina, the retinas were digested in 200 μL NP-40 cell lysis buffer (Invitrogen, Cat #: FNN0021) supplemented with protease inhibitors (Thermo Scientific, Cat #: A32955). Digestion was performed on ice for 30 minutes prior to sample centrifugation at 8,000×g for 10 minutes at 4° C. Supernatant was collected and total protein was determined using BCA protein assay (Thermo Scientific, Cat #: A53225). The protein samples were then adjusted to 50 μg total protein in 100 μL/well and assayed using a human IgG1 ELISA kit (Invitrogen, Cat #: EHIGG1). Candidate AAV2.7m8-pADV1176 showed higher expression after intravitreal injections (FIG. 6).

### Example 6—Binding Affinity of Products of AAV2.7m8-pADV1176 Using Kinetics Analysis

Supernatant from suspension HEK293 cells transfected with vector carrying gene encoding anti-C3 antibody (cell culture and transfection methods can be found in previous section) were used for kinetics analysis via Octet (BLI) to measure binding affinity to human complement C3 (Cat: C2910, Sigma). Binding experiments were performed on Octet HTX at 25° C. by LakePharma (Redwood City, CA). There were three test articles including products of pADV1176 (0.015 mg/mL), negative control (supernatant from untransfected cells) and positive control (human IgG1 in buffer—137 mM NaCl, 2.7 mM KCl, 10 mM Na₂HPO₄, 2 mM KH₂PO₄, pH 7.4, 0.98 mg/mL). The readout of Kd for the products of pADV1176 was 1.297×10⁻¹⁰ M, positive control was 2.560×10⁻¹⁰ M, and was not detectable in the negative control sample (Table 4).

### Example 7—Validating the Expression of AAV2.7m8-pADV11176 in Non-Human Primates

Intravitreal injections of AAV2.7m8-pADV1176 (1×10¹¹ vg/eye) were performed in adult (4-6 years) mauritian cynomolgus macaque (Macaca fascicularis) in Biomere (Worcester, MA) according to the protocol set-up in Table 5. On day 0, animals in group 1 and group 3 were given bilateral intravitreal (IVT) injection with formulation buffer (group 1) or AAV2.7m8-pADV1176 (group 3).

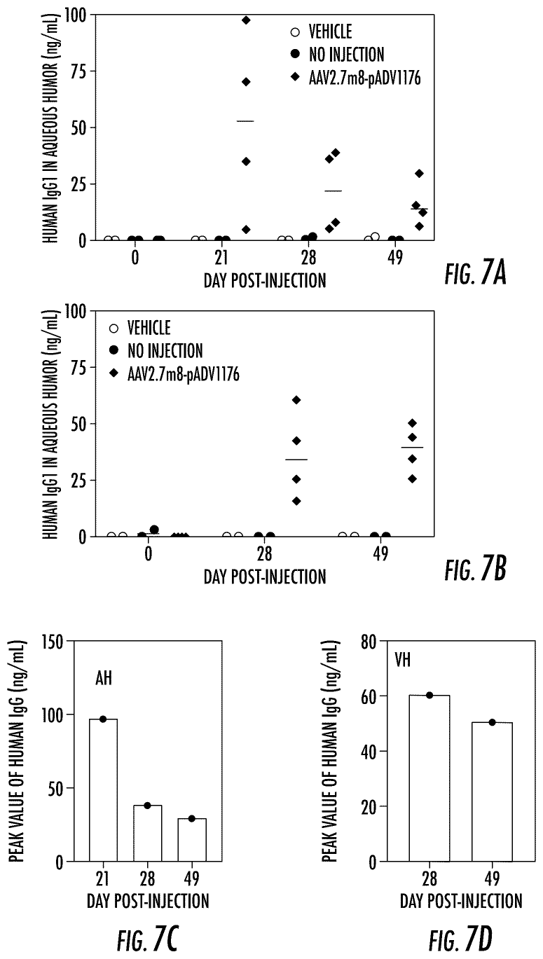

Aqueous humor (AH) and vitreous humor (VH) were collected at multiple timepoints (Table 5), in which the samples were snap-frozen on dry ice prior to storage at −80° C. To determine the levels of human IgG1 expressed after IVT injections, which correlate to the amount of anti-C3 antibody produced from AAV2.7m8-pADV1176, AH and VH samples were assayed with Human Therapeutic IgG1 ELISA Kit (Cayman Chemical, Cat #: 500910). 15 μL sample was diluted with 85 μL assay diluent per well, which duplicate wells were run for an average representative value. Kit instructions were followed except for sample incubation, which was performed for 16 hours, 4° C. with mild agitation at 500 rpm. The results indicate that AAV2.7m8-pADV1176 shows robust antibody levels in AH (3 weeks post-injection, FIG. 7A) and VH (4 weeks post injection, FIG. 7B), up to 7 weeks.

On day 62, animals in group 1 and group 3 were given bilateral IVT injections of LPS (lipopolysaccharide, 0.25 EU/eye (0.025 ng/eye), dose volume of 50 μL/eye); animals in group 2 received bilateral IVT injections of anti-C3 antibody-R21438 at 6.87 mg/eye at a dose volume of 50 μL/eye, and then immediately received bilateral IVT injections of LPS (0.25 EU/eye, equal to 0.025 ng/eye, dose volume of 50 μL/eye) (Table 6).

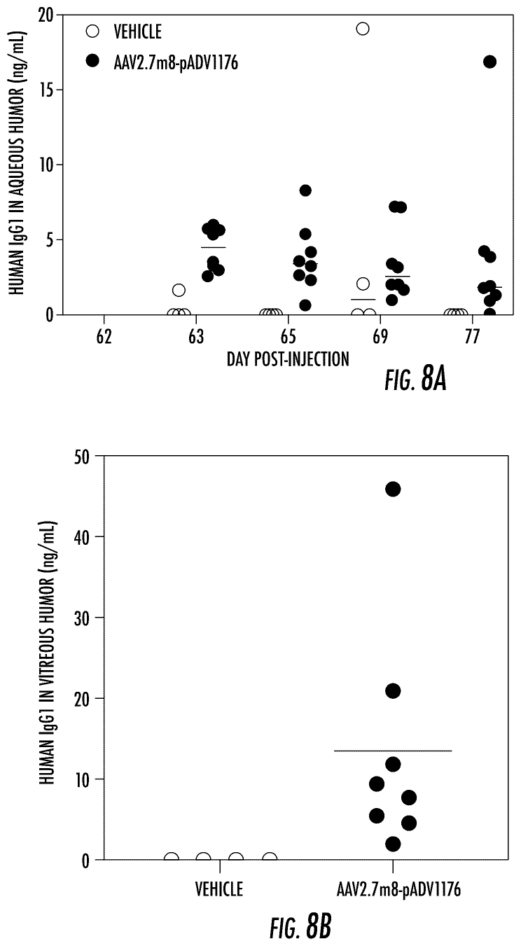

After LPS injection on day 62 (Table 6), ani-C3 antibody levels analyzed by ELISA (Cayman Chemical, Cat #: 500910) significantly decreased in AH and VH (FIG. 8A-B), but anti-C3 antibody levels in All and VH were above of limited quantification range, which was excluded in FIG. 8A-B.

TABLE 1
Complete culture media (50 mL volume)

| Reagents | Cat# | Vendors | Volume (mL) |
|---|---|---|---|
| Neurobasal A | 10888022 | Thermo Fisher Scientific | 48.45 |
| Glutamax (100x) | 35050061 | Thermo Fisher Scientific | 0.50 |
| B27 (50x) | 17504044 | Thermo Fisher Scientific | 1.00 |
| Anti-Anti (1000x) | 15240096 | Thermo Fisher Scientific | 0.05 |

TABLE 2
Transduction media (50 mL volume)

| Reagents | Cat# | Vendors | Volume (mL) |
|---|---|---|---|
| Neurobasal A | 10888022 | Thermo Fisher Scientific | 48.5 |
| Glutamax (100x) | 35050061 | Thermo Fisher Scientific | 0.5 |
| B27 (50x) | 17504044 | Thermo Fisher Scientific | 1.0 |

TABLE 3
Reagent and animal information for the mouse study (intravitreal
injection in both eyes, 2 mice per timepoint for group 1 and 2)

| Groups | Reagents | | Vol | vg/eye | D 9 | D 15 | D 21 | D 42 |
|---|---|---|---|---|---|---|---|---|
| 1 | AAV2.7m8- | | 1 μl | 2.40 × 10⁹ | 2 | 2 | 2 | 2 |
| | pADV886 | | | | | | | |
| 2 | AAV2.7m8- | | 1 μl | 2.63 × 10⁹ | 2 | 2 | 2 | 2 |
| | pADV1176 | | | | | | | |
| 3 | Formulation | Formulation buffer | 1 μl | / | / | / | / | 2 |
| 4 | Naive | No injection | / | / | / | / | / | 2 |

TABLE 4
Samples and kinetics analysis

| | Ka | Kdis | KD |
|---|---|---|---|
| Articles | (1/M s) | (1/s) | (M) |
| Products of | 2.060 × 10⁵ | 2.675 × 10⁻⁵ | 1.297 × 10⁻¹⁰ |
| pADV1176 | | | |
| Negative control | Not detectable | Not detectable | Not detectable |
| Positive control | 1.930 × 10⁵ | 4.920 × 10⁻⁵ | 2.560 × 10⁻¹⁰ |

TABLE 5
Reagent and non-human primate information for validating the expression
of AAV2.7m8-pADV1176 (intravitreal injection in both eyes)

| | IVT | | Aqueous | Vitreous |
|---|---|---|---|---|
| | in both | Volume/eye | humor (100 | humor (100 |
| ARMs | eyes | for IVT | μL/collection) | μL/collection) |
| 1 (n = 2) | Formulation | 50 μL | Baseline, day | Baseline, |
| | buffer | | 21, day 28 | day 28 |
| 2 (n = 2) | No injection | / | and day 49 | and day 49 |
| 3 (n = 4) | AAV2.7m8- | 50 μL | | |
| | pADV1176 | | | |

IVT: intravitreal injection

TABLE 6
Reagent and non-human primate (NHP) information
for validating the levels of AAV2.7m8-pADV1176
after bilateral LPS injection on Day 62

| | | Aqueous | Vitreous |
|---|---|---|---|
| ARMs | Bilateral | humor (100 | humor (100 |
| (NHPs) | IVT on Day 62 | μL/collection) | μL/collection) |
| 1 (n = 2) | LPS (50 μl/eye) | Day 63, day | Day 77 |
| 2 (n = 2) | NGM antibody (50 μl/eye) | 65, day 69 | |
| | and LPS (50 μl/eye) | and day 77 | |
| 3 (n = 4) | LPS (50 μl/eye) | | |

LPS (lipopolysaccharide): 0.25 EU/eye (0.025 ng/eye).

NGM Antibody (Anti-C3 Antibody-SR21438): 6.85 mg/Eye (50 μL).

Nucleotide and Amino Acid Sequences

| | |
|---|---|
| LOCUS | pADV881_Construct3_std_no_tags 2214 bp |
| FEATURES | Location/Qualifiers |
| gene | <1..>2214 |
| | /label = ″Construct3_std_NoTags″ |
| sig_peptide | 1..66 |
| | /label = ″Kappa signal″ |
| Light_Chain | 67..708 |
| | /label = ″light chain″ |
| Furin_linker | 709..720 |
| | /label = ″Furin″ |
| SGSG_linker | 721..732 |
| | /label = ″SGSG″ |
| SGSG_linker | 733..804 |
| | /label = ″F2A″ |
| sig_peptide | 805..870 |
| | /label = ″Kappa signal″ |
| Heavy_Chain | 871..2211 |
| | /label = ″heavy chain″ |
| STOP | 2212..2214 |
| | /label = ″STOP″ |

ORIGIN

| | |
|---|---|
| 1 | atggatatga gagtccccgc ccaactcctc ggtttgcttc tcctttggtt gagaggggcc |
| 61 | agatgcgaca tccaaatgac ccagtcgccg tcgtcgctgt cggcgtccgt cggtgatcgc |
| 121 | gtgaccatca cttgtaaagc ctcggagaac gtggacacct atgtgtcctg gtaccagcag |
| 181 | aagccgggaa aggcccccaa gctcttaatc tacggcgcat ccaaccgcta caccggagtc |
| 241 | ccgtcccgct tctccggctc gggctcggga actgacttca ccttcaccat ttcgtcactg |
| 301 | caaccagagg atattgctac ctaccactgt ggacagtcac actcctaccc cctgaccttt |
| 361 | ggtcaaggga ccaagctcga aatcaagcgg actgtggccg ccccttccgt gttcatcttc |
| 421 | cctccttccg atgagcagct caagagcgga actgcaagcg tcgtgtgcct gctgaacaac |
| 481 | ttttaccccc gggaagccaa agtgcagtgg aaagtcgaca atgccctgca atccggaaac |
| 541 | agtcaggaaa gcgtgaccga acaggactcg aaggactcca cgtactccct ttcctccacg |
| 601 | ctaaccctga gcaaggcaga ctatgagaag cacaaggtct acgcatgcga agtgacacac |
| 661 | cagggcctga gcagccctgt gaccaagagc ttcaacaggg gagagtgccg cgccaagcgc |
| 721 | agcggctccg gtgcccccgt gaagcaaacc ctgaatttcg atctgctgaa gttggcgggc |
| 781 | gacgtggaaa gcaatcccgg acccatggat atgcgcgtgc cggcccagtt gctgggactg |
| 841 | ctgctcctgt ggctgcgggg agccagatgc caagtgcagc ttgtgcagtc cggtgccgaa |
| 901 | gtcaagaaac ctggagcctc cgtcaaagtg tcctgcaagg cctcgggcta caccttcacc |
| 961 | gacttctaca tggactgggt ccgacaggcg ccagggcagc gcctggagtg gatgggctat |
| 1021 | atctacccgc acaacgcagg aaccacctac aaccagcagt tcactggaag agtgaccatt |
| 1081 | accgtcgata agtcagccag caccgcgtac atggaactgt cctctctccg gtccgaggat |
| 1141 | actgctgtgt actactgtgc gcggcggggc ggcttcgact ttgactactg gggacagggg |
| 1201 | actctggtca ctgtgtcctc cgcatccacc aagggtcctt cagtgttccc attggccccg |
| 1261 | agctcaaagt ccacatcagg gggaactgcc gctctcggat gcctcgtgaa ggattacttt |
| 1321 | cccgaacccg tgaccgtgtc ctggaactct ggcgcgctca cctccggggt gcataccttc |
| 1381 | cccgcggtgc tgcagagctc cgggctgtac tccctgtcga gcgtggtcac cgtgccatcg |
| 1441 | tcgtccctgg gaactcagac ctacatctgc aacgtgaacc acaagccatc caacaccaag |
| 1501 | gtcgacaaga aggtcgagcc gaagtcctgc gataagactc atacttgccc gccgtgccct |
| 1561 | gctccggccc ttgccggggg ccctagcgtg ttcctcttcc cacctaaacc gaaggacacc |
| 1621 | ctcatgattt cccgcactcc tgaagtgacc tgtgtggtag tggacgtgtc ccatgaggac |
| 1681 | cccgaggtca agttcaattg gtacgtggac ggcgtggaag tgcacaacgc caagaccaag |
| 1741 | ccgagggagg aacagtacaa ctcgacttac cgggtcgtgt ccgtgctgac tgtgctgcac |
| 1801 | caggactggc tcaacggcaa agagtataag tgcaaagtgt ccaacaaggc cctgcccgct |
| 1861 | ccgattgaaa agactattag caaggccaag ggacagcctc gggagccgca agtgtacacc |
| 1921 | ctgccgccat cacgggagga aatgaccaag aaccaagttt ccctgacgtg cctggtcaaa |
| 1981 | gggttctacc cctcggacat cgcggtggag tgggaatcca acggtcaacc tgaaaacaac |
| 2041 | tacaagacca ctccgcccgt gctcgactcc gacggatcgt tcttcttgta ctccaagctg |
| 2101 | accgtggaca agagccgctg gcagcagggc aacgtgttct catgttccgt gatgcatgaa |
| 2161 | gccctgcata accactacac tcagaagtcc ctgagcctgt cccccggaaa gtga (SEQ ID NO: 6) |

| | |
|---|---|
| LOCUS | pADV882_Construct3_std 2403 bp |
| FEATURES | Location/Qualifiers |
| | gene <1..>2403 |
| | /label = ″Construct3_std″ |
| sig_peptide | 1..66 |
| | /label = ″Kappa signal″ |
| light_Chain | 67..708 |
| | /label = ″light chain″ |
| (G4S)x3 | 709..753 |
| | /label = ″(G4S)x3″ |
| 3xFLAG | 754..819 |
| | /label = ″3xFLAG″ |
| Furin_linker | 820..831 |
| | /label = ″Furin″ |
| SGSG_linker | 832..843 |
| | /label = ″SGSG″ |
| F2A_linker | 844..915 |
| | /label = ″F2A″ |
| sig_peptide | 916..981 |
| | /label = ″Kappa signal″ |
| Heavy_Chain | 982..2322 |
| | /label = ″heavy chain″ |
| (G4S)x3 | 2323..2367 |
| | /label = ″(G4S)x3″ |
| myc_*_Tag | 2368..2400 |
| | /label = ″myc_*″ |
| STOP | 2401..2403 |
| | /label = ″STOP″ |

ORIGIN

| | |
|---|---|
| 1 | atggatatga gagtccccgc ccaactcctc ggtttgcttc tcctttggtt gagaggggcc |
| 61 | agatgcgaca tccaaatgac ccagtcgccg tcgtcgctgt cggcgtccgt cggtgatcgc |
| 121 | gtgaccatca cttgtaaagc ctcggagaac gtggacacct atgtgtcctg gtaccagcag |
| 181 | aagccgggaa aggcccccaa gctcttaatc tacggcgcat ccaaccgcta caccggagtc |
| 241 | ccgtcccgct tctccggctc gggctcggga actgacttca ccttcaccat ttcgtcactg |
| 301 | caaccagagg atattgctac ctaccactgt ggacagtcac actcctaccc cctgaccttt |
| 361 | ggtcaaggga ccaagctcga aatcaagcgg actgtggccg ccccttccgt gttcatcttc |
| 421 | cctccttccg atgagcagct caagagcgga actgcaagcg tcgtgtgcct gctgaacaac |
| 481 | ttttaccccc gggaagccaa agtgcagtgg aaagtcgaca atgccctgca atccggaaac |
| 541 | agtcaggaaa gcgtgaccga acaggactcg aaggactcca cgtactccct ttcctccacg |
| 601 | ctaaccctga gcaaggcaga ctatgagaag cacaaggtct acgcatgcga agtgacacac |
| 661 | cagggcctga gcagccctgt gaccaagagc ttcaacaggg gagagtgcgg agggggggga |
| 721 | tcaggagggg gtggatcagg tggtggagga tctgactaca aagaccatga tggagactac |
| 781 | aaggaccatg acattgacta caaggatgat gatgacaagc gcgccaagcg cagcggctcc |
| 841 | ggtgcccccg tgaagcaaac cctgaatttc gatctgctga agttggcggg cgacgtggaa |
| 901 | agcaatcccg gacccatgga tatgcgcgtg ccggcccagt tgctgggact gctgctcctg |
| 961 | tggctgcggg gagccagatg ccaagtgcag cttgtgcagt ccggtgccga agtcaagaaa |
| 1021 | cctggagcct ccgtcaaagt gtcctgcaag gcctcgggct acaccttcac cgacttctac |
| 1081 | atggactggg tccgacaggc gccagggcag cgcctggagt ggatgggcta tatctacccg |
| 1141 | cacaacgcag gaaccaccta caaccagcag ttcactggaa gagtgaccat taccgtcgat |
| 1201 | aagtcagcca gcaccgcgta catggaactg tcctctctcc ggtccgagga tactgctgtg |
| 1261 | tactactgtg cgcggcgggg cggcttcgac tttgactact ggggacaggg gactctggtc |
| 1321 | actgtgtcct ccgcatccac caagggtcct tcagtgttcc cattggcccc gagctcaaag |
| 1381 | tccacatcag ggggaactgc cgctctcgga tgcctcgtga aggattactt tcccgaaccc |
| 1441 | gtgaccgtgt cctggaactc tggcgcgctc acctccgggg tgcatacctt ccccgcggtg |
| 1501 | ctgcagagct ccgggctgta ctccctgtcg agcgtggtca ccgtgccatc gtcgtccctg |
| 1561 | ggaactcaga cctacatctg caacgtgaac cacaagccat ccaacaccaa ggtcgacaag |
| 1621 | aaggtcgagc cgaagtcctg cgataagact catacttgcc cgccgtgccc tgctccggcc |
| 1681 | cttgccgggg gccctagcgt gttcctcttc ccacctaaac cgaaggacac cctcatgatt |
| 1741 | tcccgcactc ctgaagtgac ctgtgtggta gtggacgtgt cccatgagga ccccgaggtc |
| 1801 | aagttcaatt ggtacgtgga cggcgtggaa gtgcacaacg ccaagaccaa gccgagggag |
| 1861 | gaacagtaca actcgactta ccgggtcgtg tccgtgctga ctgtgctgca ccaggactgg |
| 1921 | ctcaacggca aagagtataa gtgcaaagtg tccaacaagg ccctgcccgc tccgattgaa |
| 1981 | aagactatta gcaaggccaa gggacagcct cgggagccgc aagtgtacac cctgccgcca |
| 2041 | tcacgggagg aaatgaccaa gaaccaagtt tccctgacgt gcctggtcaa agggttctac |
| 2101 | ccctcggaca tcgcggtgga gtgggaatcc aacggtcaac ctgaaaacaa ctacaagacc |
| 2161 | actccgcccg tgctcgactc cgacggatcg ttcttcttgt actccaagct gaccgtggac |
| 2221 | aagagccgct ggcagcaggg caacgtgttc tcatgttccg tgatgcatga agccctgcat |
| 2281 | aaccactaca ctcagaagtc cctgagcctg tcccccggaa agggaggggg tggctcaggc |
| 2341 | ggaggcggct cgggcggcgg gggcagcgaa cagaagctga tctccgaaga ggacctgtga |
| 2401 | taa (SEQ ID NO: 7) |

| | |
|---|---|
| LOCUS | pADV883_Construct3_1.3 2403 bp |
| FEATURES | Location/Qualifiers |
| gene | <1..>2403 |
| | /label = ″Construct3_1.3″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFLAG | 754..819 |
| | /label = ″3xFLAG″ |
| Furin_linker | 820..831 |
| | /label = ″Furin″ |
| SGSG_linker | 832..843 |
| | /label = ″SGSG″ |
| F2A_linker | 844..915 |
| | /label = ″F2A″ |
| sig_peptide | 916..981 |
| | /label = ″Kappa signal″ |
| heavy_chain | 982..2322 |
| | /label = ″heavy chain″ |
| (G4S)x3 | 2323..2367 |
| | /label = ″ (G4S)x3″ |
| myc_*_tag | 2368..2400 |
| | /label = ″myc_*″ |
| STOP | 2401..2403 |
| | /label = ″STOP″ |

ORIGIN

| | |
|---|---|
| 1 | atggacatga gagtgcctgc acaacttctt ggacttctgt tgctctggtt gagaggagcc |
| 61 | cgctgtgaca tccagatgac ccagtcccca agcagcctgt cagcctcagt gggagatagg |
| 121 | gtcaccatta cttgcaaagc cagtgaaaat gtggacacct atgtgtcctg gtaccagcag |
| 181 | aagcctggca aagcacccaa gctcctcatc tatggagcct ccaaccggta cactggggtg |
| 241 | ccctcccggt tctcgggatc aggatcgggt actgacttta ccttcactat tagcagcctg |
| 301 | caacctgagg acattgccac ataccactgt ggacagtccc actcctaccc cctgaccttt |
| 361 | gggcagggca ccaagctgga gatcaagaga actgtggcag ccccctctgt gttcatcttc |
| 421 | cctccctcgg atgaacagct gaagtcaggc actgcttctg tggtctgcct cctcaacaac |
| 481 | ttctacccaa gagaggccaa ggtccagtgg aaagtggaca atgccctgca aagtggcaac |
| 541 | agccaggaat cagtgactga acaggactcc aaggacagca cttacagcct gtcctccacc |
| 601 | ctcaccctgt ccaaggctga ctatgagaag cacaaggtct atgcctgtga agtgacccac |
| 661 | cagggcttga gcagcccagt gaccaagagc ttcaacagag gggagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag gtctggatca |
| 841 | ggggccccag tgaagcagac cctcaacttt gacctcctga agctggctgg agatgtggaa |
| 901 | tccaaccctg gtcccatgga tatgagggtg ccagcccagc tcctgggcct cctgctcctg |
| 961 | tggctgaggg gtgccagatg ccaagtgcag ctggtgcagt cgggtgctga agtgaaaaag |
| 1021 | cctggagcct cagtcaaagt gtcctgcaag gcctcaggct acaccttcac tgacttctac |
| 1081 | atggactggg tccgccaagc ccctggacag cggctggagt ggatgggcta catctacccc |
| 1141 | cacaatgctg gcaccaccta caaccagcag ttcactggga gagtcaccat cacagtggac |
| 1201 | aagagtgcct ccactgcata catggaactg agcagcctca gatcagagga cactgcagtc |
| 1261 | tactactgtg ctaggagagg gggctttgac tttgactact ggggtcaagg gacccttgtc |
| 1321 | actgtgtcct cagcctccac caagggcccc tcagtgttcc ccctggcccc ctcctccaaa |
| 1381 | tccacctctg ggggaactgc agccctgggc tgcctggtca aggactactt ccctgagcct |
| 1441 | gtgactgtct cctggaactc aggagccctg acatcaggag tgcacacctt ccctgctgtg |
| 1501 | ctgcaaagct caggcttgta ctccctgtcc tctgtggtca ctgtgccaag ctccagccta |
| 1561 | ggtacccaga cctacatctg caatgtcaac cacaagccct ccaacaccaa ggtggacaag |
| 1621 | aaagtggagc ccaagtcctg tgacaagacc catacttgcc ctccctgccc tgcccctgcc |
| 1681 | ttggctggag gtccatcagt gttcctgttc ccacccaagc ccaaggatac cctcatgatc |
| 1741 | tcccgcactc ctgaagtgac ctgtgtggtg gtggatgtgt cccatgagga ccctgaagtc |
| 1801 | aagttcaatt ggtatgtgga tggagtggaa gtgcacaatg ccaagaccaa gcctagggaa |
| 1861 | gaacagtaca acagcaccta cagggttgtg tcagtgctga ctgtgctgca ccaggattgg |
| 1921 | ctcaatggca aagagtacaa gtgcaaagta tccaacaagg ccctgcctgc ccccattgaa |
| 1981 | aagaccatct ccaaggccaa gggccagccc agggaacccc aggtctacac cctgccccct |
| 2041 | tcccgggagg agatgaccaa gaaccaagtc tccctgactt gccttgtgaa gggtttctac |
| 2101 | ccctcggaca ttgcagtgga atgggagtcc aatggccagc cagagaacaa ctacaagacc |
| 2161 | acccctcctg tgctggactc agatggctcc ttcttcctgt actccaagtt aactgtggac |
| 2221 | aagtccagat ggcagcaggg caatgtgttc agctgctcag tgatgcatga agccctgcac |
| 2281 | aaccactaca cccagaagtc cctgtccctg agcccaggaa agggtggagg gggctcaggg |
| 2341 | ggaggaggct ctggaggggg aggctcagag caaaagctga tctcagagga agatctgtga |
| 2401 | taa (SEQ ID NO: 8) |

| | |
|---|---|
| LOCUS | pADV884_Construct3_0.75 2403 bp |
| FEATURES | Location/Qualifiers |
| gene | <1..>2403 |
| | /label = ″Construct3_0.75″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFlag | 754..819 |
| | /label = ″3xFlag″ |
| Furin_linker | 820..831 |
| | /label = ″Furin″ |
| SGSG_linker | 832..843 |
| | /label = ″SGSG″ |
| F2A_linker | 844..915 |
| | /label = ″F2A″ |
| sig_peptide | 916..981 |
| | /label = ″Kappa signal″ |
| heavy_chain | 982..2322 |
| | /label = ″heavy chain″ |
| (G4S)x3 | 2323..2367 |
| | /label = ″(G4S)x3″ |
| myc_*_tag | 2368..2400 |
| | /label = ″myc_*″ |
| STOP | 2401..2403 |
| | /label = ″STOP″ |

ORIGIN

| | |
|---|---|
| 1 | atggatatga gagtccctgc acaattactg ggcctgctgc tgctttggtt gaggggagcc |
| 61 | agatgtgaca ttcagatgac tcagtccccc tcctccttgt cggcctcagt gggggacagg |
| 121 | gtcaccatca cctgtaaagc ctcagagaat gtggatacct atgtctcctg gtaccagcag |
| 181 | aagcctggaa aggccccaaa gctcctgatc tatggtgcca gcaaccggta cactggtgtc |
| 241 | ccctcccgct tctcgggatc aggatcgggc actgacttca cattcaccat ttcctccctt |
| 301 | caacctgagg acattgccac ctaccattgt ggacagagcc actcctaccc ccttaccttt |
| 361 | ggccagggta ccaagctgga aatcaagaga acagtggcag ccccctctgt gttcatcttt |
| 421 | cccccttcgg atgagcagct gaagagtggc actgcttcgg tggtctgcct ccttaacaac |
| 481 | ttctaccccc gggaagccaa ggtccagtgg aaagtggaca atgccctgca gtcgggcaac |
| 541 | agccaagagt cagtgactga gcaagactcc aaggactcca cctactccct ctcctccacc |
| 601 | ctcaccctga gcaaggctga ctatgagaag cacaaggtct atgcctgtga agtgacccac |
| 661 | cagggcctga gcagcccagt gaccaagagc ttcaacaggg gagagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag atctggttcg |
| 841 | ggagcccctg tgaagcaaac cctcaacttt gacctactga agctggctgg agatgtggaa |
| 901 | tccaacccag gtcccatgga catgagggtg ccagcccagc tcctggggct gctcctcctc |
| 961 | tggctgcggg gagctaggtg ccaggtccaa ctggtgcagt caggggcaga agtcaagaag |
| 1021 | cctggtgcca gtgtgaaagt gtcctgcaaa gcctcaggat acaccttcac tgacttctac |
| 1081 | atggactggg ttcgccaggc acctggccag aggctggagt ggatgggtta catctacccc |
| 1141 | cacaacgctg gaaccaccta caaccagcag ttcactggaa gagtcaccat tactgtggat |
| 1201 | aagtctgcca gcactgccta catggaactc agctccctga gatcagagga cactgctgtg |
| 1261 | tactactgtg cccgccgggg aggctttgac tttgactact ggggccaggg caccctggtc |
| 1321 | actgtgtcct cggcctccac taagggtccg tcagtgttcc cattggcccc aagctccaag |
| 1381 | agcacctcag gaggcactgc tgccctgggc tgcttggtca aggactactt ccctgagcct |
| 1441 | gtgactgtgt cctggaactc aggggccctc acctcgggag tgcacacctt ccctgcggtg |
| 1501 | ctccagagct caggactgta cagcctgagc tcagtggtca ctgtcccttc ctcctccctg |
| 1561 | gggacccaga cctacatctg caatgtcaac cataagccct ccaacaccaa agtagacaag |
| 1621 | aaagtggagc ccaagtcctg tgataagacc cacacttgcc ctccctgccc ggcccctgcc |
| 1681 | ctggctggag gaccctcagt gttcctgttc ccacccaagc ccaaggatac cctgatgatc |
| 1741 | tcccgcactc ctgaagtgac ctgtgtggtg gtggatgtgt cccatgaaga tcctgaagtc |
| 1801 | aagttcaatt ggtatgtgga tggagtggaa gtgcacaatg caaagaccaa gccaagagaa |
| 1861 | gaacagtaca acagcaccta ccgggtggtg tcagtgctga ctgtgctgca ccaggactgg |
| 1921 | cttaatggaa aggagtacaa gtgcaaagtg tccaacaagg ccctgcctgc ccccattgaa |
| 1981 | aagaccatct ccaaggccaa gggccagccc agggaacccc aagtgtacac cctgcccccg |
| 2041 | tcccgggaag agatgaccaa gaaccaagtg tccctgactt gcctggtcaa gggcttctac |
| 2101 | ccctcggaca ttgcagtgga gtgggaaagc aatggccagc cagagaacaa ctacaagaca |
| 2161 | acccctcctg tgctggactc agatggctcc ttcttcctgt actccaagct cactgtggac |
| 2221 | aagtccagat ggcaacaggg caatgtgttc tcctgctctg tgatgcatga ggccctccac |
| 2281 | aaccactaca cccaaaagtc cttgagcctg tccccgggga agggaggggg tggctcaggg |
| 2341 | ggagggggct ctgggggtgg aggctcagaa cagaagctga tctcagaaga ggacctgtga |
| 2401 | taa (SEQ ID NO: 9) |

| | |
|---|---|
| LOCUS | pADV885_Construct3_2.5 2403 bp |
| FEATURES | Location/Qualifiers |
| gene | <1..>2403 |
| | /label = ″Construct3_2.5″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFlag | 754..819 |
| | /label = ″3xFlag″ |
| Furin_linker | 820..831 |
| | /label = ″Furin″ |
| GSGS_linker | 832..843 |
| | /label = ″GSGS″ |
| F2A_linker | 844..915 |
| | /label = ″F2A″ |
| sig_peptide | 916..981 |
| | /label = ″Kappa signal″ |
| heavy_chain | 982..2322 |
| | /label = ″heavy chain″ |
| (G4S)x3 | 2323..2367 |
| | /label = ″(G4S)x3″ |
| myc_*_tag | 2368..2400 |
| | /label = ″myc_*″ |
| STOP | 2401..2403 |
| | /label = ″STOP″ |

ORIGIN

| | |
|---|---|
| 1 | atggatatga gagtgcctgc ccaactgtta ggattgctgc tgctttggtt gaggggagcc |
| 61 | agatgtgaca tccagatgac ccagtcccca tcctccctgt ctgcatcagt gggggataga |
| 121 | gtgaccatta cttgcaaagc ctctgagaat gtggatacct atgtgtcctg gtatcaacag |
| 181 | aagccaggaa aagcccccaa actcctcatc tatggtgcaa gcaacaggta cactggagtg |
| 241 | cccagcagat tctcaggcag tggttcagga actgacttca ccttcactat cagcagcctg |
| 301 | cagcctgagg acattgccac ctaccattgt ggacagagcc actcctaccc actcaccttt |
| 361 | ggccagggca ctaagctgga gatcaagagg actgtggcag ccccctcagt ctttatcttc |
| 421 | ccacctagtg atgaacagct gaagtcaggc actgcctcag tggtttgcct gctcaacaac |
| 481 | ttctacccaa gagaagccaa ggtccagtgg aaggtggaca atgcccttca gtctggcaac |
| 541 | agccaagagt cagtgactga acaggactcc aaggactcca cctactccct ctcctccacc |
| 601 | ctcaccctga gcaaggcaga ctatgagaag cacaaggtct atgcctgtga agtgacccac |
| 661 | cagggcctga gcagccctgt caccaagagc ttcaacaggg gagagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag atcaggctct |
| 841 | ggagcccctg tgaagcagac cctcaacttt gatctgctga agctggctgg ggatgtggag |
| 901 | agcaaccctg gccccatgga catgagagtc cctgcccaat tgctgggcct ccttctcctg |
| 961 | tggcttaggg gtgctaggtg ccaagtgcag ctggtgcagt caggggctga agtgaaaaag |
| 1021 | cctggagcct cagtgaaagt gtcctgcaag gcttcagggt acaccttcac tgacttctac |
| 1081 | atggattggg tcagacaggc cccaggacag aggctggagt ggatgggcta catctacccc |
| 1141 | cacaatgcag ggaccaccta caaccagcag ttcactggaa gagtgactat cacagtggat |
| 1201 | aagtcagcct ccactgccta catggagctg tccagcctga gatcagagga cactgcagtc |
| 1261 | tactactgtg ccagaagggg aggctttgac tttgactact ggggacaggg caccctggtc |
| 1321 | actgtctcct cagcaagcac caagggtcct tcagtgttcc ccttggcccc ctcctccaag |
| 1381 | tccacctcag ggggaactgc agccctgggg tgcctggtca aggactactt ccctgagcca |
| 1441 | gtcactgtgt cctggaactc aggggccctc acctctggag tgcacacctt ccctgctgtg |
| 1501 | ctgcaaagct caggcctgta cagcctgagc tcagtggtca ctgtgccctc ctcctccctg |
| 1561 | ggtacccaaa cctacatctg caatgtcaac cacaagccct ccaacaccaa agtggacaag |
| 1621 | aaagtggagc ccaagtcctg tgacaagacc cacacatgcc ctccctgccc tgcccctgcc |
| 1681 | ctggctggag gtccctcagt gttcctgttc ccacccaagc ccaaggacac actgatgatc |
| 1741 | tccaggactc ctgaagtgac ctgtgtggta gtggatgtgt cccatgaaga tcctgaagtc |
| 1801 | aagttcaatt ggtatgtgga tggagtggaa gtgcacaatg ccaagaccaa gcccagagaa |
| 1861 | gaacagtaca acagcaccta cagagtggtg tcagtcctca ctgtgctgca ccaggactgg |
| 1921 | ctcaatggaa aggagtacaa gtgcaaagtg tccaacaagg ccctgcctgc ccccattgaa |
| 1981 | aagaccatct ccaaggccaa gggacagccc agggaacccc aggtctacac cctaccacca |
| 2041 | tccagggagg aaatgaccaa gaaccaagtg tccttgactt gccttgtgaa gggcttctac |
| 2101 | ccctctgaca ttgctgtgga atgggaatcc aatggtcaac ctgagaacaa ctacaagacc |
| 2161 | acccctcctg tgctggacag tgatggctcc ttcttcctgt actccaagct gactgtggac |
| 2221 | aagtccagat ggcagcaggg caatgtgttc agctgctcag tgatgcatga agccctccac |
| 2281 | aaccactaca cccagaagtc cctctccctg tcccctggga agggaggggg tggctcaggg |
| 2341 | ggagggggct caggaggtgg aggctcagag cagaagctga tttctgaaga ggacctgtga |
| 2401 | taa (SEQ ID NO: 10) |

| | |
|---|---|
| LOCUS | pADV886_Construct4_std_no_tags 2196 bp |
| FEATURES | Location/Qualifiers |
| gene | <1..>2196 |
| | /label = ″Construct4_std_NoTags″ |
| sig_peptide | 1..66 |
| | /label = ″Kappa signal″ |
| light_chain | 67..708 |
| | /label = ″light chain″ |
| Furin_linker | 709..720 |
| | /label = ″Furin″ |
| SGSG_linker | 721..732 |
| | /label = ″SGSG″ |
| T2A_linker | 733..786 |
| | /label = ″T2A″ |
| sig_peptide | 787..852 |
| | /label = ″Kappa signal″ |
| heavy_chain | 853..2193 |
| | /label = ″heavy chain″ |
| STOP | 2194..2196 |
| | /label = ″STOP″ |

ORIGIN

| | |
|---|---|
| 1 | atggatatga gagtccccgc ccaactcctc ggtttgcttc tcctttggtt gagaggggcc |
| 61 | agatgcgaca tccaaatgac ccagtcgccg tcgtcgctgt cggcgtccgt cggtgatcgc |
| 121 | gtgaccatca cttgtaaagc ctcggagaac gtggacacct atgtgtcctg gtaccagcag |
| 181 | aagccgggaa aggcccccaa gctcttaatc tacggcgcat ccaaccgcta caccggagtc |
| 241 | ccgtcccgct tctccggctc gggctcggga actgacttca ccttcaccat ttcgtcactg |
| 301 | caaccagagg atattgctac ctaccactgt ggacagtcac actcctaccc cctgaccttt |
| 361 | ggtcaaggga ccaagctcga aatcaagcgg actgtggccg ccccttccgt gttcatcttc |
| 421 | cctccttccg atgagcagct caagagcgga actgcaagcg tcgtgtgcct gctgaacaac |
| 481 | ttttaccccc gggaagccaa agtgcagtgg aaagtcgaca atgccctgca atccggaaac |
| 541 | agtcaggaaa gcgtgaccga acaggactcg aaggactcca cgtactccct ttcctccacg |
| 601 | ctaaccctga gcaaggcaga ctatgagaag cacaaggtct acgcatgcga agtgacacac |
| 661 | cagggcctga gcagccctgt gaccaagagc ttcaacaggg gagagtgccg cgccaagcgc |
| 721 | agcggctccg gtgaggggcg ggggagcctg ctgacatgcg gcgatgtgga ggagaacccg |
| 781 | ggcccgatgg atatgcgcgt gccggcccag ttgctgggac tgctgctcct gtggctgcgg |
| 841 | ggagccagat gccaagtgca gcttgtgcag tccggtgccg aagtcaagaa acctggagcc |
| 901 | tccgtcaaag tgtcctgcaa ggcctcgggc tacaccttca ccgacttcta catggactgg |
| 961 | gtccgacagg cgccagggca gcgcctggag tggatgggct atatctaccc gcacaacgca |
| 1021 | ggaaccacct acaaccagca gttcactgga agagtgacca ttaccgtcga taagtcagcc |
| 1081 | agcaccgcgt acatggaact gtcctctctc cggtccgagg atactgctgt gtactactgt |
| 1141 | gcgcggcggg gcggcttcga ctttgactac tggggacagg ggactctggt cactgtgtcc |
| 1201 | tccgcatcca ccaagggtcc ttcagtgttc ccattggccc cgagctcaaa gtccacatca |
| 1261 | gggggaactg ccgctctcgg atgcctcgtg aaggattact ttcccgaacc cgtgaccgtg |
| 1321 | tcctggaact ctggcgcgct cacctccggg gtgcatacct tccccgcggt gctgcagagc |
| 1381 | tccgggctgt actccctgtc gagcgtggtc accgtgccat cgtcgtccct gggaactcag |
| 1441 | acctacatct gcaacgtgaa ccacaagcca tccaacacca aggtcgacaa gaaggtcgag |
| 1501 | ccgaagtcct gcgataagac tcatacttgc ccgccgtgcc ctgctccggc ccttgccggg |
| 1561 | ggccctagcg tgttcctctt cccacctaaa ccgaaggaca ccctcatgat ttcccgcact |
| 1621 | cctgaagtga cctgtgtggt agtggacgtg tcccatgagg accccgaggt caagttcaat |
| 1681 | tggtacgtgg acggcgtgga agtgcacaac gccaagacca agccgaggga ggaacagtac |
| 1741 | aactcgactt accgggtcgt gtccgtgctg actgtgctgc accaggactg gctcaacggc |
| 1801 | aaagagtata agtgcaaagt gtccaacaag gccctgcccg ctccgattga aaagactatt |
| 1861 | agcaaggcca agggacagcc tcgggagccg caagtgtaca ccctgccgcc atcacgggag |
| 1921 | gaaatgacca agaaccaagt ttccctgacg tgcctggtca aagggttcta cccctcggac |
| 1981 | atcgcggtgg agtgggaatc caacggtcaa cctgaaaaca actacaagac cactccgccc |
| 2041 | gtgctcgact ccgacggatc gttcttcttg tactccaagc tgaccgtgga caagagccgc |
| 2101 | tggcagcagg gcaacgtgtt ctcatgttcc gtgatgcatg aagccctgca taaccactac |
| 2161 | actcagaagt ccctgagcct gtcccccgga aagtga (SEQ ID NO: 11) |

| | |
|---|---|
| LOCUS | pADV887_Construct4_std 2385 bp |
| FEATURES | Location/Qualifiers |
| gene | <1..>2385 |
| | / label = ″Construct4 std″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFlag | 754..819 |
| | /label = ″3xFlag″ |
| Furin_linker | 820..831 |
| | /label = ″Furin″ |
| GSGS_linker | 832..843 |
| | /label = ″GSGS″ |
| T2A_linker | 844..897 |
| | /label = ″T2A″ |
| sig_peptide | 898..963 |
| | /label = ″Kappa signal″ |
| heavy_chain | 964..2304 |
| | /label = ″heavy chain″ |
| (G4S)x3 | 2305..2349 |
| | /label = ″(G4S)x3″ |
| myc_*_tag | 2350..2382 |
| | /label = ″myc_*″ |
| STOP | 2383..2385 |
| | /label = ″STOP″ |

ORIGIN

| | |
|---|---|
| 1 | atggatatga gagtccccgc ccaactcctc ggtttgcttc tcctttggtt gagaggggcc |
| 61 | agatgcgaca tccaaatgac ccagtcgccg tcgtcgctgt cggcgtccgt cggtgatcgc |
| 121 | gtgaccatca cttgtaaagc ctcggagaac gtggacacct atgtgtcctg gtaccagcag |
| 181 | aagccgggaa aggcccccaa gctcttaatc tacggcgcat ccaaccgcta caccggagtc |
| 241 | ccgtcccgct tctccggctc gggctcggga actgacttca ccttcaccat ttcgtcactg |
| 301 | caaccagagg atattgctac ctaccactgt ggacagtcac actcctaccc cctgaccttt |
| 361 | ggtcaaggga ccaagctcga aatcaagcgg actgtggccg ccccttccgt gttcatcttc |
| 421 | cctccttccg atgagcagct caagagcgga actgcaagcg tcgtgtgcct gctgaacaac |
| 481 | ttttaccccc gggaagccaa agtgcagtgg aaagtcgaca atgccctgca atccggaaac |
| 541 | agtcaggaaa gcgtgaccga acaggactcg aaggactcca cgtactccct ttcctccacg |
| 601 | ctaaccctga gcaaggcaga ctatgagaag cacaaggtct acgcatgcga agtgacacac |
| 661 | cagggcctga gcagccctgt gaccaagagc ttcaacaggg gagagtgcgg agggggggga |
| 721 | tcaggagggg gtggatcagg tggtggagga tctgactaca aagaccatga tggagactac |
| 781 | aaggaccatg acattgacta caaggatgat gatgacaagc gcgccaagcg cagcggctcc |
| 841 | ggtgaggggc gggggagcct gctgacatgc ggcgatgtgg aggagaaccc gggcccgatg |
| 901 | gatatgcgcg tgccggccca gttgctggga ctgctgctcc tgtggctgcg gggagccaga |
| 961 | tgccaagtgc agcttgtgca gtccggtgcc gaagtcaaga aacctggagc ctccgtcaaa |
| 1021 | gtgtcctgca aggcctcggg ctacaccttc accgacttct acatggactg ggtccgacag |
| 1081 | gcgccagggc agcgcctgga gtggatgggc tatatctacc cgcacaacgc aggaaccacc |
| 1141 | tacaaccagc agttcactgg aagagtgacc attaccgtcg ataagtcagc cagcaccgcg |
| 1201 | tacatggaac tgtcctctct ccggtccgag gatactgctg tgtactactg tgcgcggcgg |
| 1261 | ggcggcttcg actttgacta ctggggacag gggactctgg tcactgtgtc ctccgcatcc |
| 1321 | accaagggtc cttcagtgtt cccattggcc ccgagctcaa agtccacatc agggggaact |
| 1381 | gccgctctcg gatgcctcgt gaaggattac tttcccgaac ccgtgaccgt gtcctggaac |
| 1441 | tctggcgcgc tcacctccgg ggtgcatacc ttccccgcgg tgctgcagag ctccgggctg |
| 1501 | tactccctgt cgagcgtggt caccgtgcca tcgtcgtccc tgggaactca gacctacatc |
| 1561 | tgcaacgtga accacaagcc atccaacacc aaggtcgaca agaaggtcga gccgaagtcc |
| 1621 | tgcgataaga ctcatacttg cccgccgtgc cctgctccgg cccttgccgg gggccctagc |
| 1681 | gtgttcctct tcccacctaa accgaaggac accctcatga tttcccgcac tcctgaagtg |
| 1741 | acctgtgtgg tagtggacgt gtcccatgag gaccccgagg tcaagttcaa ttggtacgtg |
| 1801 | gacggcgtgg aagtgcacaa cgccaagacc aagccgaggg aggaacagta caactcgact |
| 1861 | taccgggtcg tgtccgtgct gactgtgctg caccaggact ggctcaacgg caaagagtat |
| 1921 | aagtgcaaag tgtccaacaa ggccctgccc gctccgattg aaaagactat tagcaaggcc |
| 1981 | aagggacagc ctcgggagcc gcaagtgtac accctgccgc catcacggga ggaaatgacc |
| 2041 | aagaaccaag tttccctgac gtgcctggtc aaagggttct acccctcgga catcgcggtg |
| 2101 | gagtgggaat ccaacggtca acctgaaaac aactacaaga ccactccgcc cgtgctcgac |
| 2161 | tccgacggat cgttcttctt gtactccaag ctgaccgtgg acaagagccg ctggcagcag |
| 2221 | ggcaacgtgt tctcatgttc cgtgatgcat gaagccctgc ataaccacta cactcagaag |
| 2281 | tccctgagcc tgtcccccgg aaagggaggg ggtggctcag gcggaggcgg ctcgggcggc |
| 2341 | gggggcagcg aacagaagct gatctccgaa gaggacctgt gataa (SEQ ID NO: 12) |

| | |
|---|---|
| LOCUS | pADV888_Construct4_1.3 2385 bp |
| FEATURES | Location/Qualifiers |
| gene | <1..>2385 |
| | /label = ″Construct4_1.3″ |
| sig_peptide | 1..66 |
| | /label = ″Kappa signal″ |
| light_chain | 67..708 |
| | /label = ″light chain″ |
| (G4S)_x3 | 709..753 |
| | /label = ″(G4S)x3″ |
| 3xFLAG | 754..819 |
| | /label = ″3xFLAG″ |
| Furin_linker | 820..831 |
| | /label = ″Furin″ |
| GSGS_linker | 832..843 |
| | /label = ″GSGS″ |
| T2A_linker | 844..897 |
| | /label = ″T2A″ |
| sig_peptide | 898..963 |
| | /label = ″Kappa signal″ |
| heavy_chain | 964..2304 |
| | /label = ″heavy chain″ |
| (G4S)_x3 | 2305..2349 |
| | /label = ″(G4S)x3″ |
| myc_*_tag | 2350..2382 |
| | /label = ″myc_*″ |
| STOP | 2383..2385 |
| | /label = ″STOP″ |

ORIGIN

| | |
|---|---|
| 1 | atggacatga gagtgcctgc acaacttctt ggacttctgt tgctctggtt gagaggagcc |
| 61 | cgctgtgaca tccagatgac ccagtcccca agcagcctgt cagcctcagt gggagatagg |
| 121 | gtcaccatta cttgcaaagc cagtgaaaat gtggacacct atgtgtcctg gtaccagcag |
| 181 | aagcctggca aagcacccaa gctcctcatc tatggagcct ccaaccggta cactggggtg |
| 241 | ccctcccggt tctcgggatc aggatcgggt actgacttta ccttcactat tagcagcctg |
| 301 | caacctgagg acattgccac ataccactgt ggacagtccc actcctaccc cctgaccttt |
| 361 | gggcagggca ccaagctgga gatcaagaga actgtggcag ccccctctgt gttcatcttc |
| 421 | cctccctcgg atgaacagct gaagtcaggc actgcttctg tggtctgcct cctcaacaac |
| 481 | ttctacccaa gagaggccaa ggtccagtgg aaagtggaca atgccctgca aagtggcaac |
| 541 | agccaggaat cagtgactga acaggactcc aaggacagca cttacagcct gtcctccacc |
| 601 | ctcaccctgt ccaaggctga ctatgagaag cacaaggtct atgcctgtga agtgacccac |
| 661 | cagggcttga gcagcccagt gaccaagagc ttcaacagag gggagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag gtctggatca |
| 841 | ggggaaggga ggggtagcct gctcacctgt ggagacgtgg aggaaaaccc tggccccatg |
| 901 | gatatgaggg tgccagccca gctcctgggc ctcctgctcc tgtggctgag gggtgccaga |
| 961 | tgccaagtgc agctggtgca gtcgggtgct gaagtgaaaa agcctggagc ctcagtcaaa |
| 1021 | gtgtcctgca aggcctcagg ctacaccttc actgacttct acatggactg ggtccgccaa |
| 1081 | gcccctggac agcggctgga gtggatgggc tacatctacc cccacaatgc tggcaccacc |
| 1141 | tacaaccagc agttcactgg gagagtcacc atcacagtgg acaagagtgc ctccactgca |
| 1201 | tacatggaac tgagcagcct cagatcagag gacactgcag tctactactg tgctaggaga |
| 1261 | gggggctttg actttgacta ctggggtcaa gggacccttg tcactgtgtc ctcagcctcc |
| 1321 | accaagggcc cctcagtgtt ccccctggcc ccctcctcca aatccacctc tgggggaact |
| 1381 | gcagccctgg gctgcctggt caaggactac ttccctgagc ctgtgactgt ctcctggaac |
| 1441 | tcaggagccc tgacatcagg agtgcacacc ttccctgctg tgctgcaaag ctcaggcttg |
| 1501 | tactccctgt cctctgtggt cactgtgcca agctccagcc taggtaccca gacctacatc |
| 1561 | tgcaatgtca accacaagcc ctccaacacc aaggtggaca agaaagtgga gcccaagtcc |
| 1621 | tgtgacaaga cccatacttg ccctccctgc cctgcccctg ccttggctgg aggtccatca |
| 1681 | gtgttcctgt tcccacccaa gcccaaggat accctcatga tctcccgcac tcctgaagtg |
| 1741 | acctgtgtgg tggtggatgt gtcccatgag gaccctgaag tcaagttcaa ttggtatgtg |
| 1801 | gatggagtgg aagtgcacaa tgccaagacc aagcctaggg aagaacagta caacagcacc |
| 1861 | tacagggttg tgtcagtgct gactgtgctg caccaggatt ggctcaatgg caaagagtac |
| 1921 | aagtgcaaag tatccaacaa ggccctgcct gcccccattg aaaagaccat ctccaaggcc |
| 1981 | aagggccagc ccagggaacc ccaggtctac accctgcccc cttcccggga ggagatgacc |
| 2041 | aagaaccaag tctccctgac ttgccttgtg aagggtttct acccctcgga cattgcagtg |
| 2101 | gaatgggagt ccaatggcca gccagagaac aactacaaga ccacccctcc tgtgctggac |
| 2161 | tcagatggct ccttcttcct gtactccaag ttaactgtgg acaagtccag atggcagcag |
| 2221 | ggcaatgtgt tcagctgctc agtgatgcat gaagccctgc acaaccacta cacccagaag |
| 2281 | tccctgtccc tgagcccagg aaagggtgga gggggctcag ggggaggagg ctctggaggg |
| 2341 | ggaggctcag agcaaaagct gatctcagag gaagatctgt gataa (SEQ ID NO: 13) |

| | |
|---|---|
| LOCUS | pADV889_Construct4_0.75 2385 bp |
| FEATURES | Location/Qualifiers |
| gene | <1..>2385 |
| | /label = ″Construct4_0.75″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFlag | 754..819 |
| | /label = ″3xFlag″ |
| Furin_linker | 820..831 |
| | /label = ″Furin″ |
| GSGS_linker | 832..843 |
| | /label = ″GSGS″ |
| _T2A_linker | 844..897 |
| | /label = ″T2A″ |
| sig_peptide | 898..963 |
| | /label = ″Kappa signal″ |
| heavy_chain | 964..2304 |
| | /label = ″heavy chain″ |
| (G4S)_x3 | 2305..2349 |
| | /label = ″(G4S)x3″ |
| myc_*_tag | 2350..2382 |
| | /label = ″myc_*″ |
| STOP | 2383..2385 |
| | /label = ″STOP″ |

ORIGIN

| | |
|---|---|
| 1 | atggatatga gagtccctgc acaattactg ggcctgctgc tgctttggtt gaggggagcc |
| 61 | agatgtgaca ttcagatgac tcagtccccc tcctccttgt cggcctcagt gggggacagg |
| 121 | gtcaccatca cctgtaaagc ctcagagaat gtggatacct atgtctcctg gtaccagcag |
| 181 | aagcctggaa aggccccaaa gctcctgatc tatggtgcca gcaaccggta cactggtgtc |
| 241 | ccctcccgct tctcgggatc aggatcgggc actgacttca cattcaccat ttcctccctt |
| 301 | caacctgagg acattgccac ctaccattgt ggacagagcc actcctaccc ccttaccttt |
| 361 | ggccagggta ccaagctgga aatcaagaga acagtggcag ccccctctgt gttcatcttt |
| 421 | cccccttcgg atgagcagct gaagagtggc actgcttcgg tggtctgcct ccttaacaac |
| 481 | ttctaccccc gggaagccaa ggtccagtgg aaagtggaca atgccctgca gtcgggcaac |
| 541 | agccaagagt cagtgactga gcaagactcc aaggactcca cctactccct ctcctccacc |
| 601 | ctcaccctga gcaaggctga ctatgagaag cacaaggtct atgcctgtga agtgacccac |
| 661 | cagggcctga gcagcccagt gaccaagagc ttcaacaggg gagagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag atctggttcg |
| 841 | ggagagggaa ggggctccct cctgacttgt ggagatgtgg aagagaaccc tggccccatg |
| 901 | gacatgaggg tgccagccca gctcctgggg ctgctcctcc tctggctgcg gggagctagg |
| 961 | tgccaggtcc aactggtgca gtcaggggca gaagtcaaga agcctggtgc cagtgtgaaa |
| 1021 | gtgtcctgca aagcctcagg atacaccttc actgacttct acatggactg ggttcgccag |
| 1081 | gcacctggcc agaggctgga gtggatgggt tacatctacc cccacaacgc tggaaccacc |
| 1141 | tacaaccagc agttcactgg aagagtcacc attactgtgg ataagtctgc cagcactgcc |
| 1201 | tacatggaac tcagctccct gagatcagag gacactgctg tgtactactg tgcccgccgg |
| 1261 | ggaggctttg actttgacta ctggggccag ggcaccctgg tcactgtgtc ctcggcctcc |
| 1321 | actaagggtc cgtcagtgtt cccattggcc ccaagctcca agagcacctc aggaggcact |
| 1381 | gctgccctgg gctgcttggt caaggactac ttccctgagc ctgtgactgt gtcctggaac |
| 1441 | tcaggggccc tcacctcggg agtgcacacc ttccctgcgg tgctccagag ctcaggactg |
| 1501 | tacagcctga gctcagtggt cactgtccct tcctcctccc tggggaccca gacctacatc |
| 1561 | tgcaatgtca accataagcc ctccaacacc aaagtagaca agaaagtgga gcccaagtcc |
| 1621 | tgtgataaga cccacacttg ccctccctgc ccggcccctg ccctggctgg aggaccctca |
| 1681 | gtgttcctgt tcccacccaa gcccaaggat accctgatga tctcccgcac tcctgaagtg |
| 1741 | acctgtgtgg tggtggatgt gtcccatgaa gatcctgaag tcaagttcaa ttggtatgtg |
| 1801 | gatggagtgg aagtgcacaa tgcaaagacc aagccaagag aagaacagta caacagcacc |
| 1861 | taccgggtgg tgtcagtgct gactgtgctg caccaggact ggcttaatgg aaaggagtac |
| 1921 | aagtgcaaag tgtccaacaa ggccctgcct gcccccattg aaaagaccat ctccaaggcc |
| 1981 | aagggccagc ccagggaacc ccaagtgtac accctgcccc cgtcccggga agagatgacc |
| 2041 | aagaaccaag tgtccctgac ttgcctggtc aagggcttct acccctcgga cattgcagtg |
| 2101 | gagtgggaaa gcaatggcca gccagagaac aactacaaga caacccctcc tgtgctggac |
| 2161 | tcagatggct ccttcttcct gtactccaag ctcactgtgg acaagtccag atggcaacag |
| 2221 | ggcaatgtgt tctcctgctc tgtgatgcat gaggccctcc acaaccacta cacccaaaag |
| 2281 | tccttgagcc tgtccccggg gaagggaggg ggtggctcag ggggaggggg ctctgggggt |
| 2341 | ggaggctcag aacagaagct gatctcagaa gaggacctgt gataa (SEQ ID NO: 14) |

| | |
|---|---|
| LOCUS | pADV890_Construct4_2.5 2385 bp |
| FEATURES | Location/Qualifiers |
| gene | <1..>2385 |
| | /label = ″Construct4_2.5″ |
| sig_peptide | 1..66 |
| | /label = ″Kappa signal″ |
| light_chain | 67..708 |
| | /label = ″light chain″ |
| (G4S)x3 | 709..753 |
| | /label = ″(G4S)x3″ |
| 3xFlag | 754..819 |
| | /label = ″3xFlag″ |
| Furin_linker | 820..831 |
| | /label = ″Furin″ |
| GSGS_linker | 832..843 |
| | /label = ″GSGS″ |
| T2A_linker | 844..897 |
| | /label = ″T2A″ |
| sig_peptide | 898..963 |
| | /label = ″Kappa signal″ |
| heavy_chain | 964..2304 |
| | /label = ″heavy chain″ |
| (G4S)x3 | 2305..2349 |
| | /label = ″(G4S)x3″ |
| myc_*_tag | 2350..2382 |
| | /label = ″myc_*″ |
| STOP | 2383..2385 |
| | /label = ″STOP″ |

ORIGIN

| | |
|---|---|
| 1 | atggatatga gagtgcctgc ccaactgtta ggattgctgc tgctttggtt gaggggagcc |
| 61 | agatgtgaca tccagatgac ccagtcccca tcctccctgt ctgcatcagt gggggataga |
| 121 | gtgaccatta cttgcaaagc ctctgagaat gtggatacct atgtgtcctg gtatcaacag |
| 181 | aagccaggaa aagcccccaa actcctcatc tatggtgcaa gcaacaggta cactggagtg |
| 241 | cccagcagat tctcaggcag tggttcagga actgacttca ccttcactat cagcagcctg |
| 301 | cagcctgagg acattgccac ctaccattgt ggacagagcc actcctaccc actcaccttt |
| 361 | ggccagggca ctaagctgga gatcaagagg actgtggcag ccccctcagt ctttatcttc |
| 421 | ccacctagtg atgaacagct gaagtcaggc actgcctcag tggtttgcct gctcaacaac |
| 481 | ttctacccaa gagaagccaa ggtccagtgg aaggtggaca atgcccttca gtctggcaac |
| 541 | agccaagagt cagtgactga acaggactcc aaggactcca cctactccct ctcctccacc |
| 601 | ctcaccctga gcaaggcaga ctatgagaag cacaaggtct atgcctgtga agtgacccac |
| 661 | cagggcctga gcagccctgt caccaagagc ttcaacaggg gagagtgtgg agggggggga |
| 721 | tcaggagggg gtggatcagg tggtggagga tctgactaca aagaccatga tggagactac |
| 781 | aaggaccatg acattgacta caaggatgat gatgacaaga gagccaagag atcaggctct |
| 841 | ggagagggga ggggcagcct gctcacctgt ggagatgtgg aggaaaaccc tggccccatg |
| 901 | gacatgagag tccctgccca attgctgggc ctccttctcc tgtggcttag gggtgctagg |
| 961 | tgccaagtgc agctggtgca gtcaggggct gaagtgaaaa agcctggagc ctcagtgaaa |
| 1021 | gtgtcctgca aggcttcagg gtacaccttc actgacttct acatggattg ggtcagacag |
| 1081 | gccccaggac agaggctgga gtggatgggc tacatctacc cccacaatgc agggaccacc |
| 1141 | tacaaccagc agttcactgg aagagtgact atcacagtgg ataagtcagc ctccactgcc |
| 1201 | tacatggagc tgtccagcct gagatcagag gacactgcag tctactactg tgccagaagg |
| 1261 | ggaggctttg actttgacta ctggggacag ggcaccctgg tcactgtctc ctcagcaagc |
| 1321 | accaagggtc cttcagtgtt ccccttggcc ccctcctcca agtccacctc agggggaact |
| 1381 | gcagccctgg ggtgcctggt caaggactac ttccctgagc cagtcactgt gtcctggaac |
| 1441 | tcaggggccc tcacctctgg agtgcacacc ttccctgctg tgctgcaaag ctcaggcctg |
| 1501 | tacagcctga gctcagtggt cactgtgccc tcctcctccc tgggtaccca aacctacatc |
| 1561 | tgcaatgtca accacaagcc ctccaacacc aaagtggaca agaaagtgga gcccaagtcc |
| 1621 | tgtgacaaga cccacacatg ccctccctgc cctgcccctg ccctggctgg aggtccctca |
| 1681 | gtgttcctgt tcccacccaa gcccaaggac acactgatga tctccaggac tcctgaagtg |
| 1741 | acctgtgtgg tagtggatgt gtcccatgaa gatcctgaag tcaagttcaa ttggtatgtg |
| 1801 | gatggagtgg aagtgcacaa tgccaagacc aagcccagag aagaacagta caacagcacc |
| 1861 | tacagagtgg tgtcagtcct cactgtgctg caccaggact ggctcaatgg aaaggagtac |
| 1921 | aagtgcaaag tgtccaacaa ggccctgcct gcccccattg aaaagaccat ctccaaggcc |
| 1981 | aagggacagc ccagggaacc ccaggtctac accctaccac catccaggga ggaaatgacc |
| 2041 | aagaaccaag tgtccttgac ttgccttgtg aagggcttct acccctctga cattgctgtg |
| 2101 | gaatgggaat ccaatggtca acctgagaac aactacaaga ccacccctcc tgtgctggac |
| 2161 | agtgatggct ccttcttcct gtactccaag ctgactgtgg acaagtccag atggcagcag |
| 2221 | ggcaatgtgt tcagctgctc agtgatgcat gaagccctcc acaaccacta cacccagaag |
| 2281 | tccctctccc tgtcccctgg gaagggaggg ggtggctcag ggggaggggg ctcaggaggt |
| 2341 | ggaggctcag agcagaagct gatttctgaa gaggacctgt gataa (SEQ ID NO: 15) |

| | |
|---|---|
| LOCUS | pADV1176_C11_aC3_LC-HC_CD33-notags 2160 bp |
| FEATURES | Location/Qualifiers |
| gene | <1..>2160 |
| | /gene = ″C4_LC-HC_CD33_notags″ |
| sig_peptide | <1..48 |
| | /label = ″CD33 signal″ |
| light_chain | 49..690 |
| | /label = ″light chain″ |
| Furin_linker | 691..702 |
| | /label = ″Furin″ |
| SGSG_linker | 703..714 |
| | /label = ″SGSG″ |
| T2A_linker | 715..768 |
| | /label = ″T2A″ |
| sig_peptide | 769..816 |
| | /label = ″CD33 signal″ |
| heavy_chain | 817..2157 |
| | /label = ″heavy chain″ |
| STOP | 2158..2160 |
| | /label = ″STOP″ |

ORIGIN

| | |
|---|---|
| 1 | atgcccctgt tgctgctgct tccacttctc tgggctggtg cccttgctga catccaaatg |
| 61 | acccagtcgc cgtcgtcgct gtcggcgtcc gtcggtgatc gcgtgaccat cacttgtaaa |
| 121 | gcctcggaga acgtggacac ctatgtgtcc tggtaccagc agaagccggg aaaggccccc |
| 181 | aagctcttaa tctacggcgc atccaaccgc tacaccggag tcccgtcccg cttctccggc |
| 241 | tcgggctcgg gaactgactt caccttcacc atttcgtcac tgcaaccaga ggatattgct |
| 301 | acctaccact gtggacagtc acactcctac cccctgacct ttggtcaagg gaccaagctc |
| 361 | gaaatcaagc ggactgtggc cgccccttcc gtgttcatct tccctccttc cgatgagcag |
| 421 | ctcaagagcg gaactgcaag cgtcgtgtgc ctgctgaaca acttttaccc ccgggaagcc |
| 481 | aaagtgcagt ggaaagtcga caatgccctg caatccggaa acagtcagga aagcgtgacc |
| 541 | gaacaggact cgaaggactc cacgtactcc ctttcctcca cgctaaccct gagcaaggca |
| 601 | gactatgaga agcacaaggt ctacgcatgc gaagtgacac accagggcct gagcagccct |
| 661 | gtgaccaaga gcttcaacag gggagagtgc cgcgccaagc gcagcggctc cggtgagggg |
| 721 | cgggggagcc tgctgacatg cggcgatgtg gaggagaacc cgggcccgat gccactcctc |
| 781 | cttctgttgc ctctcctctg ggccggagct ctggcacaag tgcagcttgt gcagtccggt |
| 841 | gccgaagtca agaaacctgg agcctccgtc aaagtgtcct gcaaggcctc gggctacacc |
| 901 | ttcaccgact tctacatgga ctgggtccga caggcgccag ggcagcgcct ggagtggatg |
| 961 | ggctatatct acccgcacaa cgcaggaacc acctacaacc agcagttcac tggaagagtg |
| 1021 | accattaccg tcgataagtc agccagcacc gcgtacatgg aactgtcctc tctccggtcc |
| 1081 | gaggatactg ctgtgtacta ctgtgcgcgg cggggcggct tcgactttga ctactgggga |
| 1141 | caggggactc tggtcactgt gtcctccgca tccaccaagg gtccttcagt gttcccattg |
| 1201 | gccccgagct caaagtccac atcaggggga actgccgctc tcggatgcct cgtgaaggat |
| 1261 | tactttcccg aacccgtgac cgtgtcctgg aactctggcg cgctcacctc cggggtgcat |
| 1321 | accttccccg cggtgctgca gagctccggg ctgtactccc tgtcgagcgt ggtcaccgtg |
| 1381 | ccatcgtcgt ccctgggaac tcagacctac atctgcaacg tgaaccacaa gccatccaac |
| 1441 | accaaggtcg acaagaaggt cgagccgaag tcctgcgata agactcatac ttgcccgccg |
| 1501 | tgccctgctc cggcccttgc cgggggccct agcgtgttcc tcttcccacc taaaccgaag |
| 1561 | gacaccctca tgatttcccg cactcctgaa gtgacctgtg tggtagtgga cgtgtcccat |
| 1621 | gaggaccccg aggtcaagtt caattggtac gtggacggcg tggaagtgca caacgccaag |
| 1681 | accaagccga gggaggaaca gtacaactcg acttaccggg tcgtgtccgt gctgactgtg |
| 1741 | ctgcaccagg actggctcaa cggcaaagag tataagtgca aagtgtccaa caaggccctg |
| 1801 | cccgctccga ttgaaaagac tattagcaag gccaagggac agcctcggga gccgcaagtg |
| 1861 | tacaccctgc cgccatcacg ggaggaaatg accaagaacc aagtttccct gacgtgcctg |
| 1921 | gtcaaagggt tctacccctc ggacatcgcg gtggagtggg aatccaacgg tcaacctgaa |
| 1981 | aacaactaca agaccactcc gcccgtgctc gactccgacg gatcgttctt cttgtactcc |
| 2041 | aagctgaccg tggacaagag ccgctggcag cagggcaacg tgttctcatg ttccgtgatg |
| 2101 | catgaagccc tgcataacca ctacactcag aagtccctga gcctgtcccc cggaaagtga |
| | (SEQ ID NO: 16) |

| | |
|---|---|
| LOCUS | pADV1177_C11_aC3_HC-LC_CD33-notags 2160 bp |
| FEATURES | Location/Qualifiers |
| gene | <1..>2160 |
| | /label = ″C4_HC-LC_CD33_notags″ |
| sig_peptide | 1..48 |
| | /label = ″CD33 signal″ |
| heavy_chain | 49..1389 |
| | /label = ″heavy chain″ |
| Furin_linker | 1390..1401 |
| | /label = ″Furin″ |
| GSGS_linker | 1402..1413 |
| | /label = ″GSGS″ |
| T2A_linker | 1414..1467 |
| | /label = ″T2A″ |
| sig_peptide | 1468..1515 |
| | /label = ″CD33 signal″ |
| light_chain | 1516..2157 |
| | /label = ″light chain″ |
| STOP | 2158..2160 |
| | /label = ″STOP″ |

ORIGIN

| | |
|---|---|
| 1 | atgccactcc tccttctgtt gcctctcctc tgggccggag ctctggcaca agtgcagctt |
| 61 | gtgcagtccg gtgccgaagt caagaaacct ggagcctccg tcaaagtgtc ctgcaaggcc |
| 121 | tcgggctaca ccttcaccga cttctacatg gactgggtcc gacaggcgcc agggcagcgc |
| 181 | ctggagtgga tgggctatat ctacccgcac aacgcaggaa ccacctacaa ccagcagttc |
| 241 | actggaagag tgaccattac cgtcgataag tcagccagca ccgcgtacat ggaactgtcc |
| 301 | tctctccggt ccgaggatac tgctgtgtac tactgtgcgc ggcggggcgg cttcgacttt |
| 361 | gactactggg gacaggggac tctggtcact gtgtcctccg catccaccaa gggtccttca |
| 421 | gtgttcccat tggccccgag ctcaaagtcc acatcagggg gaactgccgc tctcggatgc |
| 481 | ctcgtgaagg attactttcc cgaacccgtg accgtgtcct ggaactctgg cgcgctcacc |
| 541 | tccggggtgc ataccttccc cgcggtgctg cagagctccg ggctgtactc cctgtcgagc |
| 601 | gtggtcaccg tgccatcgtc gtccctggga actcagacct acatctgcaa cgtgaaccac |
| 661 | aagccatcca acaccaaggt cgacaagaag gtcgagccga agtcctgcga taagactcat |
| 721 | acttgcccgc cgtgccctgc tccggccctt gccgggggcc ctagcgtgtt cctcttccca |
| 781 | cctaaaccga aggacaccct catgatttcc cgcactcctg aagtgacctg tgtggtagtg |
| 841 | gacgtgtccc atgaggaccc cgaggtcaag ttcaattggt acgtggacgg cgtggaagtg |
| 901 | cacaacgcca agaccaagcc gagggaggaa cagtacaact cgacttaccg ggtcgtgtcc |
| 961 | gtgctgactg tgctgcacca ggactggctc aacggcaaag agtataagtg caaagtgtcc |
| 1021 | aacaaggccc tgcccgctcc gattgaaaag actattagca aggccaaggg acagcctcgg |
| 1081 | gagccgcaag tgtacaccct gccgccatca cgggaggaaa tgaccaagaa ccaagtttcc |
| 1141 | ctgacgtgcc tggtcaaagg gttctacccc tcggacatcg cggtggagtg ggaatccaac |
| 1201 | ggtcaacctg aaaacaacta caagaccact ccgcccgtgc tcgactccga cggatcgttc |
| 1261 | ttcttgtact ccaagctgac cgtggacaag agccgctggc agcagggcaa cgtgttctca |
| 1321 | tgttccgtga tgcatgaagc cctgcataac cactacactc agaagtccct gagcctgtcc |
| 1381 | cccggaaagc gcgccaagcg cagcggctcc ggtgaggggc gggggagcct gctgacatgc |
| 1441 | ggcgatgtgg aggagaaccc gggcccgatg cccctgttgc tgctgcttcc acttctctgg |
| 1501 | gctggtgccc ttgctgacat ccaaatgacc cagtcgccgt cgtcgctgtc ggcgtccgtc |
| 1561 | ggtgatcgcg tgaccatcac ttgtaaagcc tcggagaacg tggacaccta tgtgtcctgg |
| 1621 | taccagcaga agccgggaaa ggcccccaag ctcttaatct acggcgcatc caaccgctac |
| 1681 | accggagtcc cgtcccgctt ctccggctcg ggctcgggaa ctgacttcac cttcaccatt |
| 1741 | tcgtcactgc aaccagagga tattgctacc taccactgtg gacagtcaca ctcctacccc |
| 1801 | ctgacctttg gtcaagggac caagctcgaa atcaagcgga ctgtggccgc cccttccgtg |
| 1861 | ttcatcttcc ctccttccga tgagcagctc aagagcggaa ctgcaagcgt cgtgtgcctg |
| 1921 | ctgaacaact tttacccccg ggaagccaaa gtgcagtgga aagtcgacaa tgccctgcaa |
| 1981 | tccggaaaca gtcaggaaag cgtgaccgaa caggactcga aggactccac gtactccctt |
| 2041 | tcctccacgc taaccctgag caaggcagac tatgagaagc acaaggtcta cgcatgcgaa |
| 2101 | gtgacacacc agggcctgag cagccctgtg accaagagct tcaacagggg agagtgctga |
| | (SEQ ID NO: 18) |

| | |
|---|---|
| LOCUS | pADV1178_C11_aC3_LC-HC_CD5-notags 2208 bp |
| FEATURES | Location/Qualifiers |
| gene | <1..>2208 |
| | /label = ″C4_LC-HC_CD5_notags″ |
| sig_peptide | 1..72 |
| | /label = ″CD5 signal″ |
| light_chain | 73..714 |
| | /label = ″light chain″ |
| Furin_linker | 715..726 |
| | /label = ″Furin″ |
| SGSG_linker | 727..738 |
| | /label = ″SGSG″ |
| T2A_linker | 739..792 |
| | /label = ″T2A″ |
| sig_peptide | 793..864 |
| | /label = ″CD5 signal″ |
| heavy_chain | 865..2205 |
| | /label = ″heavy chain″ |
| STOP | 2206..2208 |
| | /label = ″STOP″ |

ORIGIN

| | |
|---|---|
| 1 | atgcctatgg gatccttgca acctctggcc accctctacc tccttggcat gcttgtggca |
| 61 | tcagtgctgg ctgacatcca aatgacccag tcgccgtcgt cgctgtcggc gtccgtcggt |
| 121 | gatcgcgtga ccatcacttg taaagcctcg gagaacgtgg acacctatgt gtcctggtac |
| 181 | cagcagaagc cgggaaaggc ccccaagctc ttaatctacg gcgcatccaa ccgctacacc |
| 241 | ggagtcccgt cccgcttctc cggctcgggc tcgggaactg acttcacctt caccatttcg |
| 301 | tcactgcaac cagaggatat tgctacctac cactgtggac agtcacactc ctaccccctg |
| 361 | acctttggtc aagggaccaa gctcgaaatc aagcggactg tggccgcccc ttccgtgttc |
| 421 | atcttccctc cttccgatga gcagctcaag agcggaactg caagcgtcgt gtgcctgctg |
| 481 | aacaactttt acccccggga agccaaagtg cagtggaaag tcgacaatgc cctgcaatcc |
| 541 | ggaaacagtc aggaaagcgt gaccgaacag gactcgaagg actccacgta ctccctttcc |
| 601 | tccacgctaa ccctgagcaa ggcagactat gagaagcaca aggtctacgc atgcgaagtg |
| 661 | acacaccagg gcctgagcag ccctgtgacc aagagcttca acaggggaga gtgccgcgcc |
| 721 | aagcgcagcg gctccggtga ggggcggggg agcctgctga catgcggcga tgtggaggag |
| 781 | aacccgggcc cgatgcctat gggatccctc caacccctcg ccaccctcta tctcttggga |
| 841 | atgcttgttg ccagtgtgct tgcccaagtg cagcttgtgc agtccggtgc cgaagtcaag |
| 901 | aaacctggag cctccgtcaa agtgtcctgc aaggcctcgg gctacacctt caccgacttc |
| 961 | tacatggact gggtccgaca ggcgccaggg cagcgcctgg agtggatggg ctatatctac |
| 1021 | ccgcacaacg caggaaccac ctacaaccag cagttcactg gaagagtgac cattaccgtc |
| 1081 | gataagtcag ccagcaccgc gtacatggaa ctgtcctctc tccggtccga ggatactgct |
| 1141 | gtgtactact gtgcgcggcg gggcggcttc gactttgact actggggaca ggggactctg |
| 1201 | gtcactgtgt cctccgcatc caccaagggt ccttcagtgt tcccattggc cccgagctca |
| 1261 | aagtccacat cagggggaac tgccgctctc ggatgcctcg tgaaggatta ctttcccgaa |
| 1321 | cccgtgaccg tgtcctggaa ctctggcgcg ctcacctccg gggtgcatac cttccccgcg |
| 1381 | gtgctgcaga gctccgggct gtactccctg tcgagcgtgg tcaccgtgcc atcgtcgtcc |
| 1441 | ctgggaactc agacctacat ctgcaacgtg aaccacaagc catccaacac caaggtcgac |
| 1501 | aagaaggtcg agccgaagtc ctgcgataag actcatactt gcccgccgtg ccctgctccg |
| 1561 | gcccttgccg ggggccctag cgtgttcctc ttcccaccta aaccgaagga caccctcatg |
| 1621 | atttcccgca ctcctgaagt gacctgtgtg gtagtggacg tgtcccatga ggaccccgag |
| 1681 | gtcaagttca attggtacgt ggacggcgtg gaagtgcaca acgccaagac caagccgagg |
| 1741 | gaggaacagt acaactcgac ttaccgggtc gtgtccgtgc tgactgtgct gcaccaggac |
| 1801 | tggctcaacg gcaaagagta taagtgcaaa gtgtccaaca aggccctgcc cgctccgatt |
| 1861 | gaaaagacta ttagcaaggc caagggacag cctcgggagc cgcaagtgta caccctgccg |
| 1921 | ccatcacggg aggaaatgac caagaaccaa gtttccctga cgtgcctggt caaagggttc |
| 1981 | tacccctcgg acatcgcggt ggagtgggaa tccaacggtc aacctgaaaa caactacaag |
| 2041 | accactccgc ccgtgctcga ctccgacgga tcgttcttct tgtactccaa gctgaccgtg |
| 2101 | gacaagagcc gctggcagca gggcaacgtg ttctcatgtt ccgtgatgca tgaagccctg |
| 2161 | cataaccact acactcagaa gtccctgagc ctgtcccccg gaaagtga (SEQ ID NO: 19) |

| | |
|---|---|
| LOCUS | pADV1179_C11_aC3_HC-LC_CD5-notags 2208 bp |
| FEATURES | Location/Qualifiers |
| gene | <1..>2208 |
| | /label = ″C4_HC-LC_CD5_notags″ |
| sig_peptide | 1..72 |
| | /label = ″CD5 signal″ |
| heavy_chain | 73..1413 |
| | /label = ″heavy chain″ |
| Furin_linker | 1414..1425 |
| | /label = ″Furin″ |
| SGSG_linker | 1426..1437 |
| | /label = ″SGSG″ |
| T2A_linker | 1438..1491 |
| | /label = ″T2A″ |
| sig_peptide | 1492..1563 |
| | /label = ″CD5 signal″ |
| light_chain | 1564..2205 |
| | /label = ″light chain″ |
| STOP | 2206..2208 |
| | /label = ″STOP″ |

ORIGIN

| | |
|---|---|
| 1 | atgcctatgg gatccctcca acccctcgcc accctctatc tcttgggaat gcttgttgcc |
| 61 | agtgtgcttg cccaagtgca gcttgtgcag tccggtgccg aagtcaagaa acctggagcc |
| 121 | tccgtcaaag tgtcctgcaa ggcctcgggc tacaccttca ccgacttcta catggactgg |
| 181 | gtccgacagg cgccagggca gcgcctggag tggatgggct atatctaccc gcacaacgca |
| 241 | ggaaccacct acaaccagca gttcactgga agagtgacca ttaccgtcga taagtcagcc |
| 301 | agcaccgcgt acatggaact gtcctctctc cggtccgagg atactgctgt gtactactgt |
| 361 | gcgcggcggg gcggcttcga ctttgactac tggggacagg ggactctggt cactgtgtcc |
| 421 | tccgcatcca ccaagggtcc ttcagtgttc ccattggccc cgagctcaaa gtccacatca |
| 481 | gggggaactg ccgctctcgg atgcctcgtg aaggattact ttcccgaacc cgtgaccgtg |
| 541 | tcctggaact ctggcgcgct cacctccggg gtgcatacct tccccgcggt gctgcagagc |
| 601 | tccgggctgt actccctgtc gagcgtggtc accgtgccat cgtcgtccct gggaactcag |
| 661 | acctacatct gcaacgtgaa ccacaagcca tccaacacca aggtcgacaa gaaggtcgag |
| 721 | ccgaagtcct gcgataagac tcatacttgc ccgccgtgcc ctgctccggc ccttgccggg |
| 781 | ggccctagcg tgttcctctt cccacctaaa ccgaaggaca ccctcatgat ttcccgcact |
| 841 | cctgaagtga cctgtgtggt agtggacgtg tcccatgagg accccgaggt caagttcaat |
| 901 | tggtacgtgg acggcgtgga agtgcacaac gccaagacca agccgaggga ggaacagtac |
| 961 | aactcgactt accgggtcgt gtccgtgctg actgtgctgc accaggactg gctcaacggc |
| 1021 | aaagagtata agtgcaaagt gtccaacaag gccctgcccg ctccgattga aaagactatt |
| 1081 | agcaaggcca agggacagcc tcgggagccg caagtgtaca ccctgccgcc atcacgggag |
| 1141 | gaaatgacca agaaccaagt ttccctgacg tgcctggtca aagggttcta cccctcggac |
| 1201 | atcgcggtgg agtgggaatc caacggtcaa cctgaaaaca actacaagac cactccgccc |
| 1261 | gtgctcgact ccgacggatc gttcttcttg tactccaagc tgaccgtgga caagagccgc |
| 1321 | tggcagcagg gcaacgtgtt ctcatgttcc gtgatgcatg aagccctgca taaccactac |
| 1381 | actcagaagt ccctgagcct gtcccccgga aagcgcgcca agcgcagcgg ctccggtgag |
| 1441 | gggcggggga gcctgctgac atgcggcgat gtggaggaga acccgggccc gatgcctatg |
| 1501 | ggatccttgc aacctctggc caccctctac ctccttggca tgcttgtggc atcagtgctg |
| 1561 | gctgacatcc aaatgaccca gtcgccgtcg tcgctgtcgg cgtccgtcgg tgatcgcgtg |
| 1621 | accatcactt gtaaagcctc ggagaacgtg gacacctatg tgtcctggta ccagcagaag |
| 1681 | ccgggaaagg cccccaagct cttaatctac ggcgcatcca accgctacac cggagtcccg |
| 1741 | tcccgcttct ccggctcggg ctcgggaact gacttcacct tcaccatttc gtcactgcaa |
| 1801 | ccagaggata ttgctaccta ccactgtgga cagtcacact cctaccccct gacctttggt |
| 1861 | caagggacca agctcgaaat caagcggact gtggccgccc cttccgtgtt catcttccct |
| 1921 | ccttccgatg agcagctcaa gagcggaact gcaagcgtcg tgtgcctgct gaacaacttt |
| 1981 | tacccccggg aagccaaagt gcagtggaaa gtcgacaatg ccctgcaatc cggaaacagt |
| 2041 | caggaaagcg tgaccgaaca ggactcgaag gactccacgt actccctttc ctccacgcta |
| 2101 | accctgagca aggcagacta tgagaagcac aaggtctacg catgcgaagt gacacaccag |
| 2161 | ggcctgagca gccctgtgac caagagcttc aacaggggag agtgctga (SEQ ID NO: 20) |

| | |
|---|---|
| LOCUS | pADV1180_C11_aC3_LC-HC_flt1-notags 2220 bp |
| FEATURES | Location/Qualifiers |
| gene | <1..>2220 |
| | /label = ″C4_LC-HC_flt1_notags″ |
| sig_peptide | 1..78 |
| | /label = ″FLT1 signal″ |
| light_chain | 79..720 |
| | /label = ″light chain″ |
| Furin_linker | 721..732 |
| | /label = ″Furin″ |
| SGSG_linker | 733..744 |
| | /label = ″SGSG″ |
| T2A_linker | 745..798 |
| | /label = ″T2A″ |
| sig_peptide | 799..876 |
| | /label = ″FLT1 signal″ |
| heavy_chain | 877..2217 |
| | /label = ″heavy chain″ |
| STOP | 2218..2220 |
| | /label = ″STOP″ |

ORIGIN

| | |
|---|---|
| 1 | atggtgtcat actgggatac tggagtcctg ctttgtgccc tgctttcctg cctgctgttg |
| 61 | actggctcct cctcgggaga catccaaatg acccagtcgc cgtcgtcgct gtcggcgtcc |
| 121 | gtcggtgatc gcgtgaccat cacttgtaaa gcctcggaga acgtggacac ctatgtgtcc |
| 181 | tggtaccagc agaagccggg aaaggccccc aagctcttaa tctacggcgc atccaaccgc |
| 241 | tacaccggag tcccgtcccg cttctccggc tcgggctcgg gaactgactt caccttcacc |
| 301 | atttcgtcac tgcaaccaga ggatattgct acctaccact gtggacagtc acactcctac |
| 361 | cccctgacct ttggtcaagg gaccaagctc gaaatcaagc ggactgtggc cgccccttcc |
| 421 | gtgttcatct tccctccttc cgatgagcag ctcaagagcg gaactgcaag cgtcgtgtgc |
| 481 | ctgctgaaca acttttaccc ccgggaagcc aaagtgcagt ggaaagtcga caatgccctg |
| 541 | caatccggaa acagtcagga aagcgtgacc gaacaggact cgaaggactc cacgtactcc |
| 601 | ctttcctcca cgctaaccct gagcaaggca gactatgaga agcacaaggt ctacgcatgc |
| 661 | gaagtgacac accagggcct gagcagccct gtgaccaaga gcttcaacag gggagagtgc |
| 721 | cgcgccaagc gcagcggctc cggtgagggg cgggggagcc tgctgacatg cggcgatgtg |
| 781 | gaggagaacc cgggcccgat ggtgtcatat tgggatactg gagtccttct ctgtgctttg |
| 841 | ctctcctgcc tcctcctcac tggatccagc tcgggacaag tgcagcttgt gcagtccggt |
| 901 | gccgaagtca agaaacctgg agcctccgtc aaagtgtcct gcaaggcctc gggctacacc |
| 961 | ttcaccgact tctacatgga ctgggtccga caggcgccag ggcagcgcct ggagtggatg |
| 1021 | ggctatatct acccgcacaa cgcaggaacc acctacaacc agcagttcac tggaagagtg |
| 1081 | accattaccg tcgataagtc agccagcacc gcgtacatgg aactgtcctc tctccggtcc |
| 1141 | gaggatactg ctgtgtacta ctgtgcgcgg cggggcggct tcgactttga ctactgggga |
| 1201 | caggggactc tggtcactgt gtcctccgca tccaccaagg gtccttcagt gttcccattg |
| 1261 | gccccgagct caaagtccac atcaggggga actgccgctc tcggatgcct cgtgaaggat |
| 1321 | tactttcccg aacccgtgac cgtgtcctgg aactctggcg cgctcacctc cggggtgcat |
| 1381 | accttccccg cggtgctgca gagctccggg ctgtactccc tgtcgagcgt ggtcaccgtg |
| 1441 | ccatcgtcgt ccctgggaac tcagacctac atctgcaacg tgaaccacaa gccatccaac |
| 1501 | accaaggtcg acaagaaggt cgagccgaag tcctgcgata agactcatac ttgcccgccg |
| 1561 | tgccctgctc cggcccttgc cgggggccct agcgtgttcc tcttcccacc taaaccgaag |
| 1621 | gacaccctca tgatttcccg cactcctgaa gtgacctgtg tggtagtgga cgtgtcccat |
| 1681 | gaggaccccg aggtcaagtt caattggtac gtggacggcg tggaagtgca caacgccaag |
| 1741 | accaagccga gggaggaaca gtacaactcg acttaccggg tcgtgtccgt gctgactgtg |
| 1801 | ctgcaccagg actggctcaa cggcaaagag tataagtgca aagtgtccaa caaggccctg |
| 1861 | cccgctccga ttgaaaagac tattagcaag gccaagggac agcctcggga gccgcaagtg |
| 1921 | tacaccctgc cgccatcacg ggaggaaatg accaagaacc aagtttccct gacgtgcctg |
| 1981 | gtcaaagggt tctacccctc ggacatcgcg gtggagtggg aatccaacgg tcaacctgaa |
| 2041 | aacaactaca agaccactcc gcccgtgctc gactccgacg gatcgttctt cttgtactcc |
| 2101 | aagctgaccg tggacaagag ccgctggcag cagggcaacg tgttctcatg ttccgtgatg |
| 2161 | catgaagccc tgcataacca ctacactcag aagtccctga gcctgtcccc cggaaagtga |
| | (SEQ ID NO: 21) |

| | |
|---|---|
| LOCUS | pADV1181_C11_aC3_HC-LC_flt1-notags 2220 bp |
| FEATURES | Location/Qualifiers |
| gene | <1..>2220 |
| | /label = ″C4_HC-LC_flt1_notags″ |
| sig_peptide | 1..78 |
| | /label = ″FLT1 signal″ |
| heavy_chain | 79..1419 |
| | /label = ″heavy chain″ |
| Furin_linker | 1420..1431 |
| | /label = ″Furin″ |
| SGSG_linker | 1432..1443 |
| | /label = ″SGSG″ |
| T2A_linker | 1444..1497 |
| | /label = ″T2A″ |
| sig_peptide | 1498..1575 |
| | /label = ″FLT1 signal″ |
| light_chain | 1576..2217 |
| | /label = ″light chain″ |
| STOP | 2218..2220 |
| | /label = ″STOP″ |

ORIGIN

| | |
|---|---|
| 1 | atggtgtcat attgggatac tggagtcctt ctctgtgctt tgctctcctg cctcctcctc |
| 61 | actggatcca gctcgggaca agtgcagctt gtgcagtccg gtgccgaagt caagaaacct |
| 121 | ggagcctccg tcaaagtgtc ctgcaaggcc tcgggctaca ccttcaccga cttctacatg |
| 181 | gactgggtcc gacaggcgcc agggcagcgc ctggagtgga tgggctatat ctacccgcac |
| 241 | aacgcaggaa ccacctacaa ccagcagttc actggaagag tgaccattac cgtcgataag |
| 301 | tcagccagca ccgcgtacat ggaactgtcc tctctccggt ccgaggatac tgctgtgtac |
| 361 | tactgtgcgc ggcggggcgg cttcgacttt gactactggg gacaggggac tctggtcact |
| 421 | gtgtcctccg catccaccaa gggtccttca gtgttcccat tggccccgag ctcaaagtcc |
| 481 | acatcagggg gaactgccgc tctcggatgc ctcgtgaagg attactttcc cgaacccgtg |
| 541 | accgtgtcct ggaactctgg cgcgctcacc tccggggtgc ataccttccc cgcggtgctg |
| 601 | cagagctccg ggctgtactc cctgtcgagc gtggtcaccg tgccatcgtc gtccctggga |
| 661 | actcagacct acatctgcaa cgtgaaccac aagccatcca acaccaaggt cgacaagaag |
| 721 | gtcgagccga agtcctgcga taagactcat acttgcccgc cgtgccctgc tccggccctt |
| 781 | gccgggggcc ctagcgtgtt cctcttccca cctaaaccga aggacaccct catgatttcc |
| 841 | cgcactcctg aagtgacctg tgtggtagtg gacgtgtccc atgaggaccc cgaggtcaag |
| 901 | ttcaattggt acgtggacgg cgtggaagtg cacaacgcca agaccaagcc gagggaggaa |
| 961 | cagtacaact cgacttaccg ggtcgtgtcc gtgctgactg tgctgcacca ggactggctc |
| 1021 | aacggcaaag agtataagtg caaagtgtcc aacaaggccc tgcccgctcc gattgaaaag |
| 1081 | actattagca aggccaaggg acagcctcgg gagccgcaag tgtacaccct gccgccatca |
| 1141 | cgggaggaaa tgaccaagaa ccaagtttcc ctgacgtgcc tggtcaaagg gttctacccc |
| 1201 | tcggacatcg cggtggagtg ggaatccaac ggtcaacctg aaaacaacta caagaccact |
| 1261 | ccgcccgtgc tcgactccga cggatcgttc ttcttgtact ccaagctgac cgtggacaag |
| 1321 | agccgctggc agcagggcaa cgtgttctca tgttccgtga tgcatgaagc cctgcataac |
| 1381 | cactacactc agaagtccct gagcctgtcc cccggaaagc gcgccaagcg cagcggctcc |
| 1441 | ggtgaggggc gggggagcct gctgacatgc ggcgatgtgg aggagaaccc gggcccgatg |
| 1501 | gtgtcatact gggatactgg agtcctgctt tgtgccctgc tttcctgcct gctgttgact |
| 1561 | ggctcctcct cgggagacat ccaaatgacc cagtcgccgt cgtcgctgtc ggcgtccgtc |
| 1621 | ggtgatcgcg tgaccatcac ttgtaaagcc tcggagaacg tggacaccta tgtgtcctgg |
| 1681 | taccagcaga agccgggaaa ggcccccaag ctcttaatct acggcgcatc caaccgctac |
| 1741 | accggagtcc cgtcccgctt ctccggctcg ggctcgggaa ctgacttcac cttcaccatt |
| 1801 | tcgtcactgc aaccagagga tattgctacc taccactgtg gacagtcaca ctcctacccc |
| 1861 | ctgacctttg gtcaagggac caagctcgaa atcaagcgga ctgtggccgc cccttccgtg |
| 1921 | ttcatcttcc ctccttccga tgagcagctc aagagcggaa ctgcaagcgt cgtgtgcctg |
| 1981 | ctgaacaact tttacccccg ggaagccaaa gtgcagtgga aagtcgacaa tgccctgcaa |
| 2041 | tccggaaaca gtcaggaaag cgtgaccgaa caggactcga aggactccac gtactccctt |
| 2101 | tcctccacgc taaccctgag caaggcagac tatgagaagc acaaggtcta cgcatgcgaa |
| 2161 | gtgacacacc agggcctgag cagccctgtg accaagagct tcaacagggg agagtgctga |
| | (SEQ ID NO: 22) |

| | |
|---|---|
| LOCUS | pADV1182_C11_aC3_HC-LC_IgK-notags 2196 bp |
| FEATURES | Location/Qualifiers |
| gene | <1..>2196 |
| | /label = ″C4_HC-LC_IgK_notags″ |
| sig_peptide | 1..66 |
| | /label = ″Kappa signal″ |
| heavy_chain | 67..1407 |
| | /label = ″heavy chain″ |
| Furin_linker | 1408..1419 |
| | /label = ″Furin″ |
| SGSG_linker | 1420..1431 |
| | /label = ″SGSG″ |
| T2A_linker | 1432..1485 |
| | /label = ″T2A″ |
| sig_peptide | 1486..1551 |
| | /label = ″Kappa signal″ |
| light_chain | 1552..2193 |
| | /label = ″light chain″ |
| STOP | 2194..2196 |
| | /label = ″STOP″ |

ORIGIN

| | |
|---|---|
| 1 | atggatatgc gcgtgccggc ccagttgctg ggactgctgc tcctgtggct gcggggagcc |
| 61 | agatgccaag tgcagcttgt gcagtccggt gccgaagtca agaaacctgg agcctccgtc |
| 121 | aaagtgtcct gcaaggcctc gggctacacc ttcaccgact tctacatgga ctgggtccga |
| 181 | caggcgccag ggcagcgcct ggagtggatg ggctatatct acccgcacaa cgcaggaacc |
| 241 | acctacaacc agcagttcac tggaagagtg accattaccg tcgataagtc agccagcacc |
| 301 | gcgtacatgg aactgtcctc tctccggtcc gaggatactg ctgtgtacta ctgtgcgcgg |
| 361 | cggggcggct tcgactttga ctactgggga caggggactc tggtcactgt gtcctccgca |
| 421 | tccaccaagg gtccttcagt gttcccattg gccccgagct caaagtccac atcaggggga |
| 481 | actgccgctc tcggatgcct cgtgaaggat tactttcccg aacccgtgac cgtgtcctgg |
| 541 | aactctggcg cgctcacctc cggggtgcat accttccccg cggtgctgca gagctccggg |
| 601 | ctgtactccc tgtcgagcgt ggtcaccgtg ccatcgtcgt ccctgggaac tcagacctac |
| 661 | atctgcaacg tgaaccacaa gccatccaac accaaggtcg acaagaaggt cgagccgaag |
| 721 | tcctgcgata agactcatac ttgcccgccg tgccctgctc cggcccttgc cgggggccct |
| 781 | agcgtgttcc tcttcccacc taaaccgaag gacaccctca tgatttcccg cactcctgaa |
| 841 | gtgacctgtg tggtagtgga cgtgtcccat gaggaccccg aggtcaagtt caattggtac |
| 901 | gtggacggcg tggaagtgca caacgccaag accaagccga gggaggaaca gtacaactcg |
| 961 | acttaccggg tcgtgtccgt gctgactgtg ctgcaccagg actggctcaa cggcaaagag |
| 1021 | tataagtgca aagtgtccaa caaggccctg cccgctccga ttgaaaagac tattagcaag |
| 1081 | gccaagggac agcctcggga gccgcaagtg tacaccctgc cgccatcacg ggaggaaatg |
| 1141 | accaagaacc aagtttccct gacgtgcctg gtcaaagggt tctacccctc ggacatcgcg |
| 1201 | gtggagtggg aatccaacgg tcaacctgaa aacaactaca agaccactcc gcccgtgctc |
| 1261 | gactccgacg gatcgttctt cttgtactcc aagctgaccg tggacaagag ccgctggcag |
| 1321 | cagggcaacg tgttctcatg ttccgtgatg catgaagccc tgcataacca ctacactcag |
| 1381 | aagtccctga gcctgtcccc cggaaagcgc gccaagcgca gcggctccgg tgaggggcgg |
| 1441 | gggagcctgc tgacatgcgg cgatgtggag gagaacccgg gcccgatgga tatgagagtc |
| 1501 | cccgcccaac tcctcggttt gcttctcctt tggttgagag gggccagatg cgacatccaa |
| 1561 | atgacccagt cgccgtcgtc gctgtcggcg tccgtcggtg atcgcgtgac catcacttgt |
| 1621 | aaagcctcgg agaacgtgga cacctatgtg tcctggtacc agcagaagcc gggaaaggcc |
| 1681 | cccaagctct taatctacgg cgcatccaac cgctacaccg gagtcccgtc ccgcttctcc |
| 1741 | ggctcgggct cgggaactga cttcaccttc accatttcgt cactgcaacc agaggatatt |
| 1801 | gctacctacc actgtggaca gtcacactcc taccccctga cctttggtca agggaccaag |
| 1861 | ctcgaaatca agcggactgt ggccgcccct tccgtgttca tcttccctcc ttccgatgag |
| 1921 | cagctcaaga gcggaactgc aagcgtcgtg tgcctgctga acaactttta cccccgggaa |
| 1981 | gccaaagtgc agtggaaagt cgacaatgcc ctgcaatccg gaaacagtca ggaaagcgtg |
| 2041 | accgaacagg actcgaagga ctccacgtac tccctttcct ccacgctaac cctgagcaag |
| 2101 | gcagactatg agaagcacaa ggtctacgca tgcgaagtga cacaccaggg cctgagcagc |
| 2161 | cctgtgacca agagcttcaa caggggagag tgctga (SEQ ID NO: 23) |

For the 17 cassettes, amino acid sequence of heavy chain (447 aa):

| | |
|---|---|
| 1 | QVQLVQSGAE VKKPGASVKV SCKASGYTFT DFYMDWVRQA PGQRLEWMGY IYPHNAGTTY |
| 61 | NQQFTGRVTI TVDKSASTAY MELSSLRSED TAVYYCARRG GFDFDYWGQG TLVTVSSAST |
| 121 | KGPSVFPLAP SSKSTSGGTA ALGCLVKDYF PEPVTVSWNS GALTSGVHTF PAVLQSSGLY |
| 181 | SLSSVVTVPS SSLGTQTYIC NVNHKPSNTK VDKKVEPKSC DKTHTCPPCP APALAGGPSV |
| 241 | FLFPPKPKDT LMISRTPEVT CVVVDVSHED PEVKFNWYVD GVEVHNAKTK PREEQYNSTY |
| 301 | RVVSVLTVLH QDWLNGKEYK CKVSNKALPA PIEKTISKAK GQPREPQVYT LPPSREEMTK |
| 361 | NQVSLTCLVK GFYPSDIAVE WESNGQPENN YKTTPPVLDS DGSFFLYSKL TVDKSRWQQG |
| 421 | NVFSCSVMHE ALHNHYTQKS LSLSPGK (SEQ ID NO: 24) |

For the 17 cassettes, amino acid sequence of light chain (214 aa):

| | |
|---|---|
| 1 | DIQMTQSPSS LSASVGDRVT ITCKASENVD TYVSWYQQKP GKAPKLLIYG ASNRYTGVPS |
| 61 | RFSGSGSGTD FTFTISSLQP EDIATYHCGQ SHSYPLTFGQ GTKLEIKRTV AAPSVFIFPP |
| 121 | SDEQLKSGTA SVVCLLNNFY PREAKVQWKV DNALQSGNSQ ESVTEQDSKD STYSLSSTLT |
| 181 | LSKADYEKHK VYACEVTHQG LSSPVTKSFN RGEC (SEQ ID NO: 25) |

Signal peptide of CD33:
MPLLLLLPLLWAGALA (SEQ ID NO: 26)
Signal peptide of Kappa:
MDMRVPAQLLGLLLLWLRGARC (SEQ ID NO: 27)
Signal peptide of CD5:
MPMGSLQPLATLYLLGMLVASVLA (SEQ ID NO: 28)
Signal peptide of FLT1:
MVSYWDTGVLLCALLSCLLLTGSSSG (SEQ ID NO: 29)

Those skilled in the art will recognize or be able to ascertain using no more than routine experimentation, many equivalents to the specific embodiments of the invention specifically described herein.

1. A method for providing a transgene encoding an anti-Component 3 (C3) gene product to an ocular cell in a subject, comprising administering to one or more sites within the eye of the subject an effective amount of a recombinant adeno-associated virus (rAAV) comprising said transgene, wherein the effective amount is an amount sufficient to at least partially block C3 activity in the subject and transduce the ocular cell, and wherein the transduced ocular cell expresses the gene product.

2. The method of claim 1, wherein the anti-C3 gene product is an anti-C3 antibody.

3. The method of claim 1, wherein the rAAV is an AAV2.7m8, AAV2.5T.LSV1 or AAV2.R100.

4-14. (canceled)

15. A method for treating an ocular disease or disorder in a subject in need thereof, comprising administering to one or more sites within an eye of the subject an effective amount of a recombinant adeno-associated virus (rAAV), comprising a transgene encoding an anti-C3 gene product, wherein the effective amount is an amount sufficient to at least partially block C3 activity in the subject and transduce ocular cells within the subject, and wherein the transduced ocular cells express the therapeutic gene product.

16. The method of claim 15, wherein the anti-C3 gene product is an anti-C3 antibody.

17. The method of claim 15, wherein the rAAV is an AAV2.7m8, AAV2.5T.LSV1 or AAV2.R100.

18. The method of claim 15, wherein the subject is a mammalian subject.

19. The method of claim 15, wherein the effective amount is at least about 1×10¹⁰ vg, at least about 5×10¹⁰ vg, at least about 6×10¹⁰ vg, or at least about 2×10¹¹ vg.

20-23. (canceled)

24. The method of claim 15, wherein the rAAV is administered retinally, subretinally, and/or intravitreally.

25. The method of claim 15, wherein the ocular disease or disorder is glaucoma, retinitis pigmentosa, macular degeneration, wet macular generation, dry macular degeneration, retinoschisis, Leber's Congenital Amaurosis, diabetic retinopathy, achromotopsia, geographic atrophy associated dry AMD, or color blindness.

26-28. (canceled)

29. The method of claim 15, wherein the gene product is an anti-angiogenic polypeptide, a vascular endothelial growth factor (VEGF)-binding protein, anti-C3 molecule, or an opsin protein.

30. A first recombinant adeno-associated virus (rAAV) and a second rAAV use in a method for treating an ocular disease or disorder in a subject in need thereof, the method comprising:
(a) administering to a first eye of the subject a first effective amount of the first rAAV, the first rAAV comprising a first transgene encoding a first gene product;
(b) waiting for a period of time; and
(c) administering to a second eye of the subject a second effective amount of the second rAAV, the second rAAV comprising a second transgene encoding a second gene product, wherein the first and second effective amounts are amounts sufficient to transduce cells of the eye, and wherein the transduced cells express the first and second gene products, wherein either of the first or second rAAV expresses an anti-C3 gene product.

31. The first rAAV and the second rAAV of claim 30, wherein the anti-C3 gene product of either the first or second rAAV is an anti-C3 antibody.

32. The first rAAV and the second rAAV of claim 30, wherein the first rAAV and the second rAAV:
(i) are the same serotype;
(ii) comprise the same capsid proteins;
(iii) are AAV2.7m8, AAV2.5T.LSV1, or AAV2.R100;
(iv) are different serotypes; or
(v) comprise different serotypes.

33-36. (canceled)

37. The first rAAV and the second rAAV of claim 30, wherein the period of time is selected from the following: at least one week, at least one month, at least three months, at least six months, at least one year, at least 18 months, at least two years, at least three years, or longer than three years.

38. The first rAAV and the second rAAV of claim 30, wherein the subject is not administered a recombinant rAAV during the period of time.

39-40. (canceled)

41. The first rAAV and the second rAAV of claim 30, wherein the first or second gene product is a therapeutic gene product.

42. The first rAAV and the second rAAV of claim 41, wherein the first gene product and the second gene product are independently selected from: an anti-angiogenic polypeptide, a vascular endothelial growth factor (VEGF)-binding protein, an anti-VEGF agent, an anti-C3 molecule, or an opsin protein.

43. The first rAAV and the second rAAV of claim 30, wherein the ocular disease or disorder is glaucoma, retinitis pigmentosa, macular degeneration, wet macular degeneration, dry macular degeneration, retinoschisis, Leber's Congenital Amaurosis, diabetic retinopathy, achromotopsia, geographic atrophy associated with dry AMD, or color blindness.

44-46. (canceled)

47. The first rAAV and the second rAAV of claim 30, wherein the first effective amount is at least about 1×10¹⁰ vg, at least about 5×10¹⁰ vg or at least about 2×10¹¹ vg; and wherein the second effective amount is at least about 1×10¹⁰ vg, at least about 5×10¹⁰ vg, or at least about 2×10¹¹ vg.

48-52. (canceled)

53. The first rAAV and the second rAAV of claim 30, wherein:
(i) the first effective amount comprises up to about 5×10¹¹ vector genomes of the first rAAV;
(ii) the first effective amount comprises between about 1×10¹¹ and about 5×10¹¹ vector genomes of the first rAAV: or
(iii) the first effective amount comprises up to about 5×10¹¹ vector genomes of the first rAAV, and the second effective amount comprises at least about 1×10¹⁰ vector genomes of the second rAAV.

54-56. (canceled)

57. The first rAAV and the second rAAV of claim 30, wherein the first effective amount is lower than the second effective amount.

58. The first rAAV and the second rAAV of claim 30, wherein:
(i) the subject is not administered an immunosuppressant prior to, concurrent with, or following administration of the first rAAV;
(ii) the subject is administered an immunosuppressant prior to, concurrent with, or following administration of the first rAAV;
(iii) an immunosuppressant is not administered to the subject after the administration of the first rAAV;
(iv) an immunosuppressant is administered to the subject after the administration of the first rAAV and before or concurrent with the administration of the second rAAV; or
(v) an immunosuppressant is administered to the subject after the administration of the second rAAV.

59-62. (canceled)

63. The first rAAV and the second rAAV claim 30, wherein the administering of the first effective amount and the administering of the second effective amount is by intraocular injection or intravitreal injection.

64-70. (canceled)
